(19) United States
(12) Patent Application Publication
(10) Pub. No.: US 2023/0052740 A1
(43) Pub. Date: Feb. 16, 2023

(54) MODULATORS OF CIRCADIAN RHYTHMS AND USES THEREOF

(71) Applicants: The Regents of the University of California, Oakland, CA (US); The University of North Carolina at Chapel Hill, Chapel Hill, NC (US); The Research Foundation for the State University of New York, Amherst, NC (US)

(72) Inventors: Brian K. Shoichet, San Francisco, CA (US); Reed M. Stein, San Francisco, CA (US); Margarita L. Dubocovich, Clarence, NY (US); Grant C. Glatfelter, Baltimore, MD (US); Bryan L. Roth, Durham, NC (US); Hye Jin Kang, Chapel HIll, NC (US)

(21) Appl. No.: 17/638,172

(22) PCT Filed: Aug. 27, 2020
(86) PCT No.: PCT/US2020/048233

§ 371 Date: Feb. 24, 2022

Related U.S. Application Data
(60) Provisional application No. 62/893,115, filed on Aug. 28, 2019.

(51) Int. Cl. A61K 31/435; A61P 25/20

(52) U.S. Cl. CPC A61K 31/435; A61P 25/20

(57) ABSTRACT

Disclosed herein are, inter alia, compounds modulating MT₁ and MT₂ receptors' activity and methods of use thereof for treating MT₁ and MT₂ receptor-related conditions.

### CROSS-REFERENCES TO RELATED APPLICATIONS

This application claims the benefit of U.S. provisional application No. 62/893,115, filed Aug. 28, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. U24 DK116195, R21 ES023684, TR001412 and TR001413 awarded by The National Institutes of Health. The government has certain rights in the invention.

### BACKGROUND

The biological clock located within the suprachiasmatic nucleus (SCN) times the near 24 hr oscillations in neuroendocrine function. Melatonin, released primarily by the pineal gland following a circadian rhythm with high levels at night, feedbacks onto the SCN to modulate clock phase (Gillette & Mitchell, Cell Tissue Res 2002, 309:99-107). In mammals, melatonin accelerates re-entrainment to a new-dark onset and shifts clock phase at temporally distinct times, i.e. dusk and dawn (Lewy et al., Chronobiol Int 1998, 15:71-83; Skene D. J., J Neuroendocrinol 2003, 15:438-441; Cassone et al., Physiol Behav 1986, 36:1111-1121; Redman et al., Science 1983, 219:1089-1091). Pharmacological studies suggest that the melatonin-mediated phase shifts of the circadian rhythm of wheel running activity in the C3H/HeN mouse in vivo (Benloucif & Dubocovich, J Biol Rhythms 1996; 11:113-125; Dubocovich et al. FASEB J 1998; 12:1211-1220) and of neuronal firing in the rat SCN brain slice in vitro [McArthur et al., Endocrinology 1997; 138:627-634; Hunt et al. Am J Physiol Cell Physiol 2001; 280:C110-C118; Gerdin et al., FASEB J 2004; 18:1646-1656] are mediated through activation of melatonin receptors.

In the mammalian SCN, melatonin activates at least two membrane bound G-protein coupled receptors, the MT₁ and the MT₂, which mediate a number of functional responses (Masana & Dubocovich, Sci STKE 2001; 2001:E39; Dubocovich et al. Front Biosci 2003; 8:d1093-d1108). In the mouse SCN, melatonin inhibits neuronal firing (Liu et al., Neuron 1997; 19:91-102) and PACAP (Pituitary Adenylate Cyclase Activating Polypeptide)-mediated CREB (cyclic AMP-responsive element-binding protein) phosphorylation (Jin et al., Mol Cell Biol 2003; 23:1054-1060; Von Gall et al. Neuroreport 2000; 11:1803-1807) through activation of MT₁ melatonin receptors as these effects are not observed in the SCN from MT₁ knockout (KO) mice. The melatonin receptor antagonist 4P-PDOT blocked the melatonin-mediated phase advance of circadian rhythm of wheel running activity in C3H/HeN mice (Dubocovich, 1998, supra) and of neuronal firing generated in rat SCN brain slices (Hunt, supra). These results suggest the involvement of MT₂ melatonin receptor activation in mediating phase shifts of circadian rhythms in rodent models.

The disruption of circadian rhythms lead to many pathologies including sleep disorders and depression. Drugs that modulate MT₁ and the MT₂ are used to treat these conditions. Examples of marketed melatonin receptor actings drugs include Ramelteon, Agomelatine, and Tasimelteon.

Recent stidues have shown that type selective melatonin inverse agonists and agonists have a potential for resetting the circadian clock and thus to modulate sleep/wake cycles. Given a lack of selective MT₁ receptor ligands and a very limited number of selective MT₂ receptor ligands, there is an unmet need for therapeutic agents capable of modulating these targets. The proposed compounds have the potential to deliver potent, small molecule melatonin type selective receptor agonists and inverse agonists.

### BRIEF SUMMARY

Provided herein, inter alia, are small molecule agonists of melatonin type 2 (MT₂) receptor and small molecule inverse agonists of melatonin type 1 (MT₁) receptor, pharmaceutical compositions comprising these compounds, and the use of these compounds for the treatment of MT₂ receptor-related and MT₁ receptor-related conditions.

In an aspect, provided herein is a compound having the formula (I):

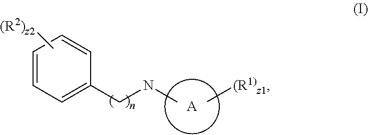

or a pharmaceutically acceptable salt thereof, wherein n is and integer from 0 to 5; z1 is an integer from 0 to 2; z2 is an integer from 0 to 5; Ring A is a substituted or unsubstituted aryl or substituted or unsubstituted heteroaryl; R¹ is independently halogen, —CX¹₃, —CHX₂, —CH₂X¹, —OCX¹₃, —OCHX¹₂, —OCH₂X¹, —CN, —SR^{1A}, —S(O)₂R^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is independently halogen, —CX²₃, —CHX²₂, —CH₂X², —OCX²₃, —OCHX²₂,
—OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A},
—NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl, substituted or unsubstituted aryl, or substituted or unsubstituted heteroaryl; R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl, substituted or unsubstituted aryl, or substituted or unsubstituted heteroaryl; and X¹ and X² are independently halogen.

In another aspect, provided herein is a compound having the formula (II):

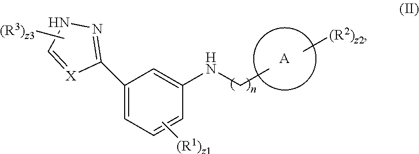

or a pharmaceutically acceptable salt thereof, wherein ring A, R¹, R², and n are as defined above; z1 is an integer from 0 to 2; z2 is an integer from 0 to 5. R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NR^{3A}R^{3B}, —C(O)R^{3A}, —C(O)—OR^{3A}, —C(O)NR^{3A}R^{3B}, —C(O)N—NR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{3A} and R^{3B} substituents bonded to the same nitrogen atom may optionally be joined to form a substituted or unsubstituted heterocycloalkyl, substituted or unsubstituted aryl, or substituted or unsubstituted heteroaryl. X³ is independently halogen. z3 is an integer from 0 to 2.

In another aspect, provided herein is a pharmaceutical composition including a pharmaceutically acceptable excipient and a compound of formula (I).

In another aspect, provided herein is a pharmaceutical composition including a pharmaceutically acceptable excipient and a compound of formula (II).

In one aspect, provided herein is a method of increasing MT₂ receptor activity in a subject in need thereof, the method comprising administering to said subject an effective amount of a compound of formula (I).

In another aspect, provided herein is a method of treating depression in a subject in need thereof, the method comprising administering to said subject an effective amount of a compound of formula (I).

In another aspect, provided herein is a method of treating an MT₂ receptor-related condition in a subject in need thereof, the method comprising administering to said subject an effective amount of a compound of formula (I).

In another aspect, provided is a method of advancing circadian phase, the method comprising administering to a subject in need thereof an effective amount of an inverse agonist of MT1 receptor of formula (II).

In another aspect, provided herein is a method of decreasing of MT₁ receptor activity in a subject in need thereof, the method comprising administering to said subject an effective amount of a compound of formula (II).

In another aspect, provided herein is a method of treating an MT₁ receptor-related condition in a subject in need thereof, the method comprising administering to said subject an effective amount of a compound of formula (II).

### DETAILED DESCRIPTION OF THE DRAWINGS

The patent or application file contains at least one drawings executed in color. Copies of this patent or patent application publication with color drawings will be provided by the Office upon request and payment of the necessary fee.

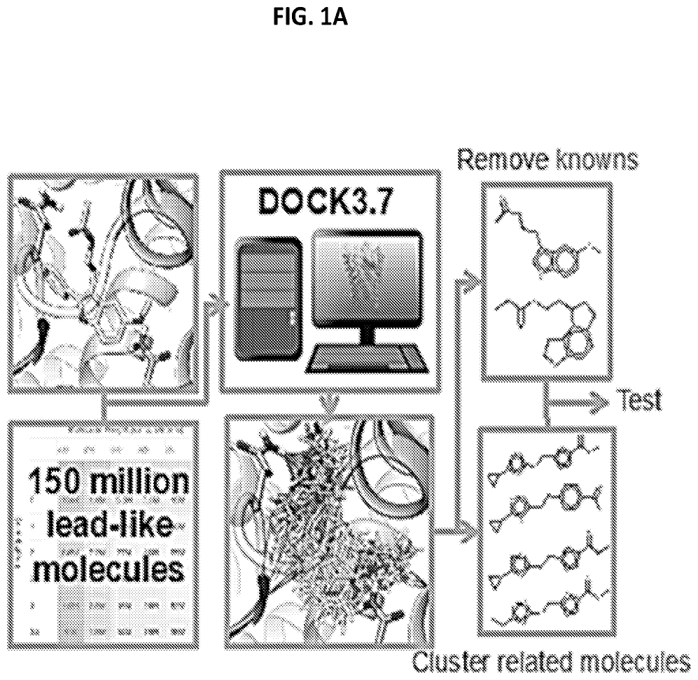

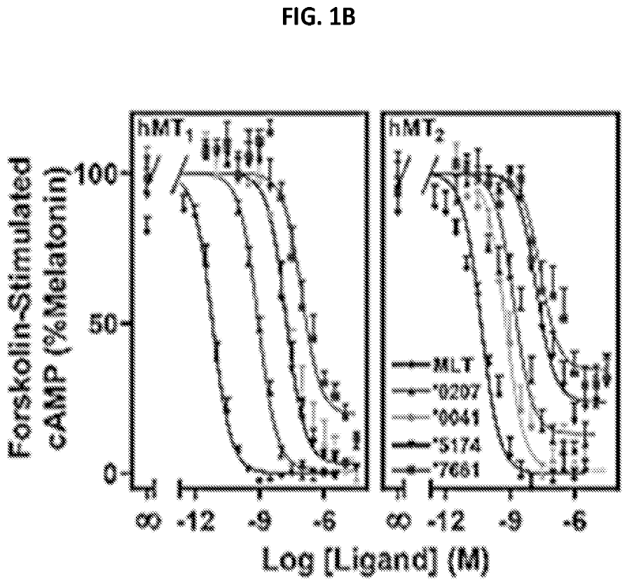

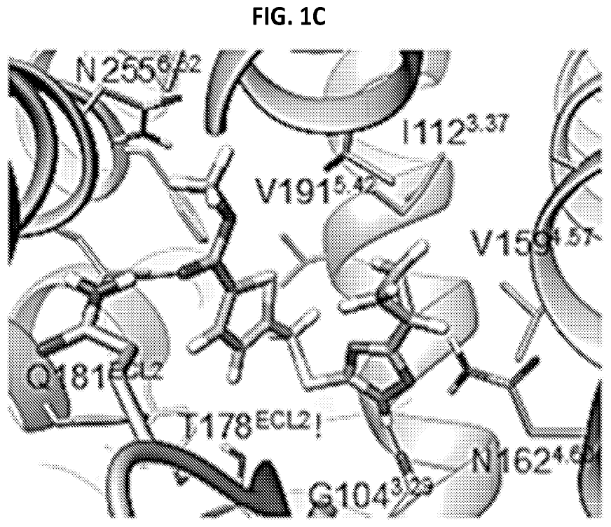

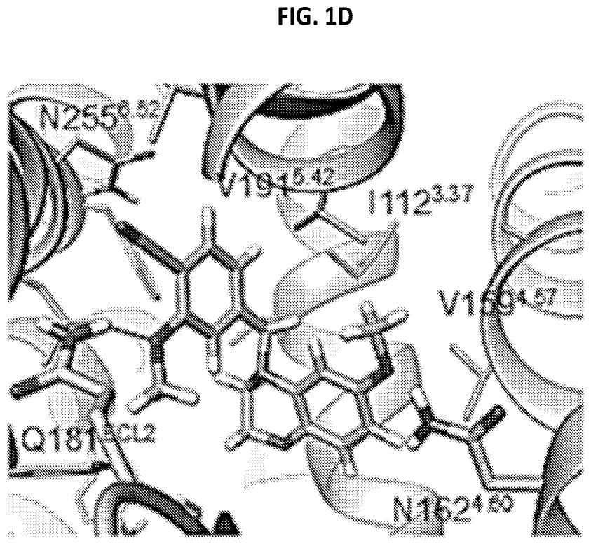

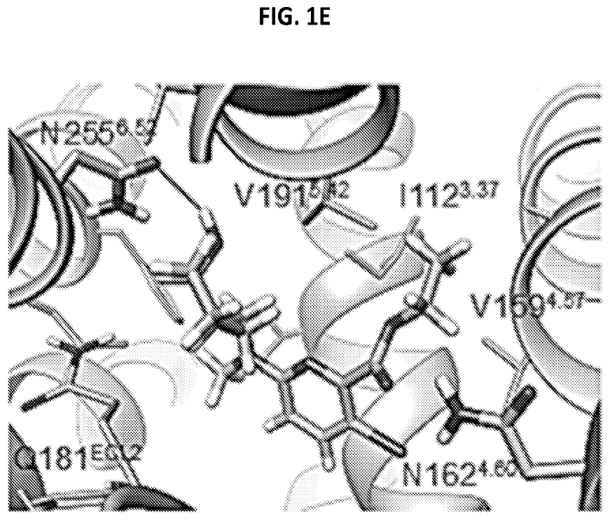

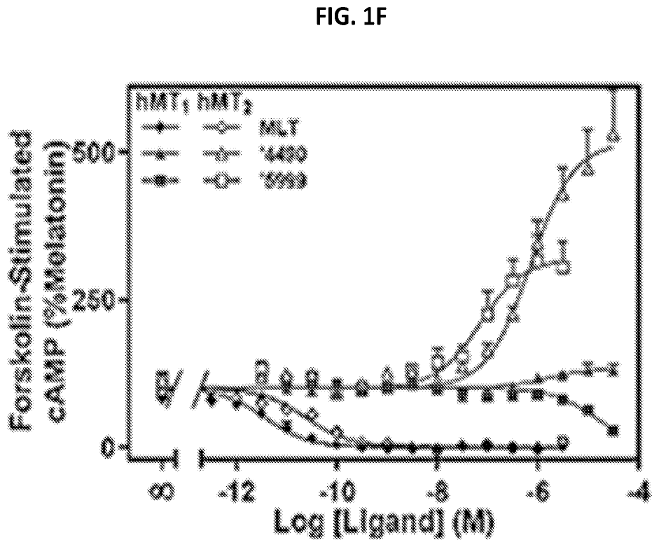

FIGS. 1A-AF. Large library docking finds novel, potent melatonin receptor ligands. FIG. 1A. Structure-based docking finds new melatonin receptor chemotypes from large make-on-demand libraries. FIG. 1B. Activation of hMT₁ and hMT₂ by melatonin and the new agonists '0207, '0041, '5174, and '7661. Data normalized to effect of isoproterenol alone represent mean±S.E.M of three independent determinations run in triplicate. FIG. 1C. Docked pose of '0207, an hMT₁/hMT₂ nonselective agonist with low nanomolar activity. FIG. 1D. Docked pose of '0041, an agonist with low nanomolar activity at MT₁ and mid-picomolar activity at MT₂. FIG. 1E. Docked pose of '4490, an MT₂-selective inverse agonist active in the high nanomolar range. FIG. 1F. Compound '4490 is a mid-nanomolar MT₂-selective inverse agonist, while '5999 is a mixed mid-nanomolar MT₁ agonist and low nanomolar MT₂ inverse-agonist. Data normalized to effect of forskolin alone represent mean±S.E.M of three independent determinations run in triplicate.

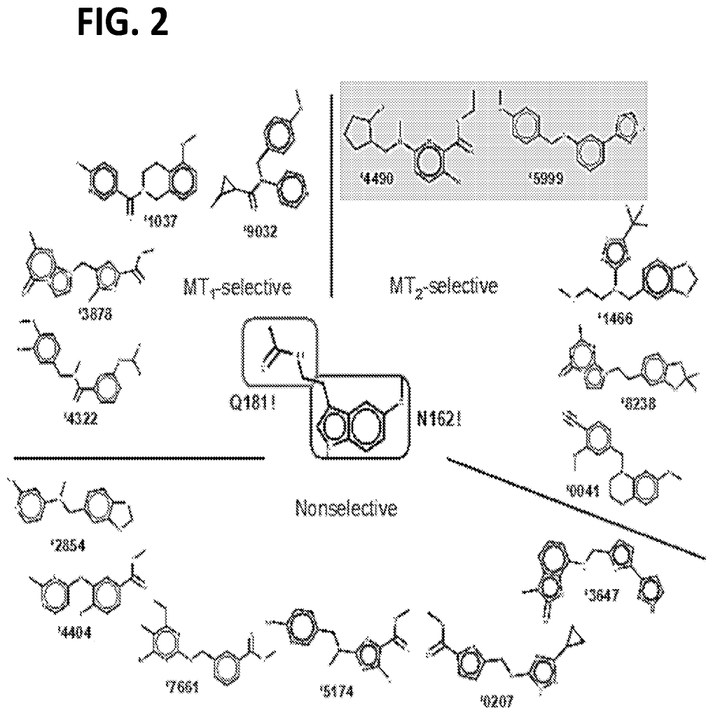

FIG. 2. Docking finds a wide range of melatonin receptor ligands, topologically unrelated to melatonin. The initial 15 docking hits are shown, highlighting groups that correspond to melatonin's acetamide side chain (blue) and its 5-methoxy-indole in their docked poses and receptor interactions. Shaded molecules are inverse agonists.

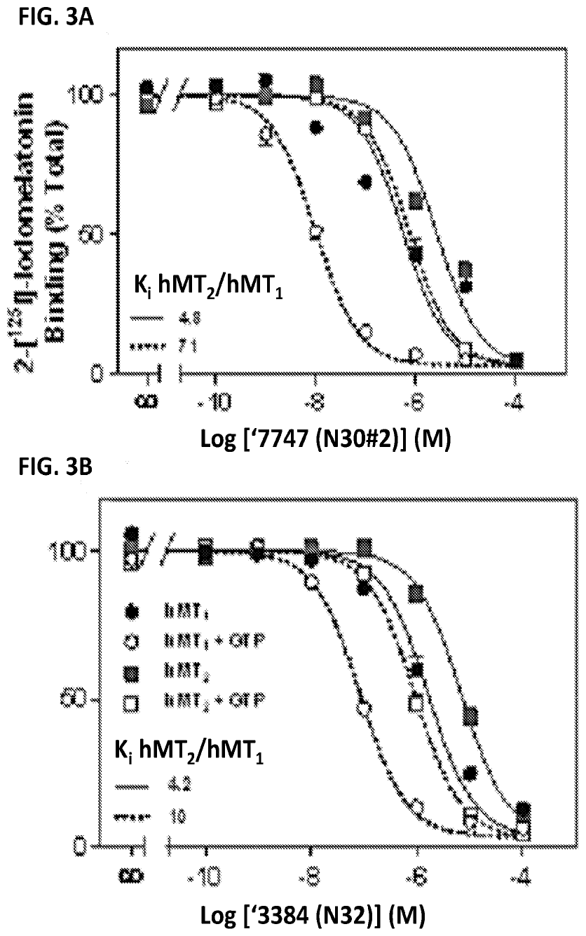

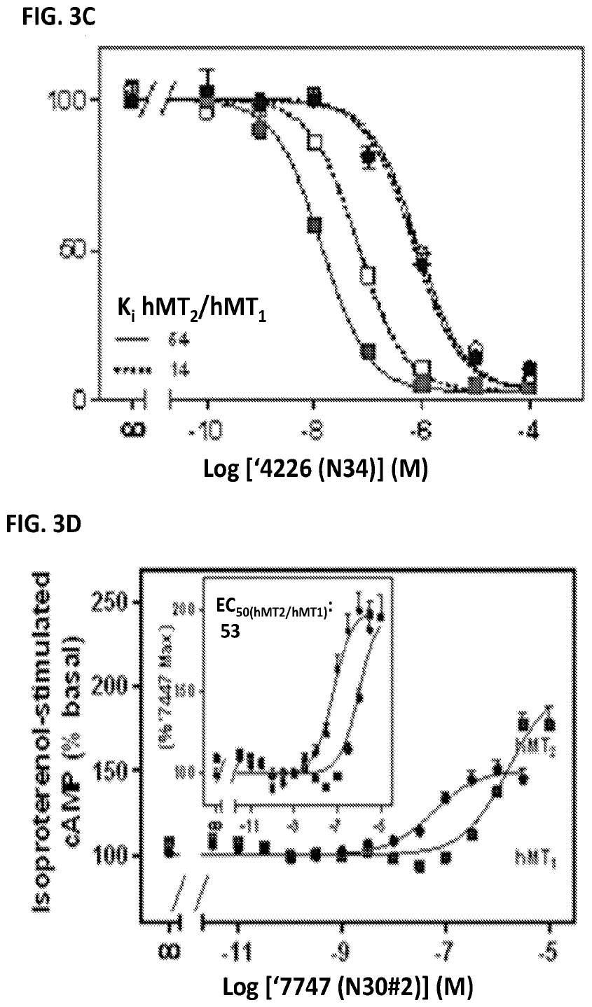

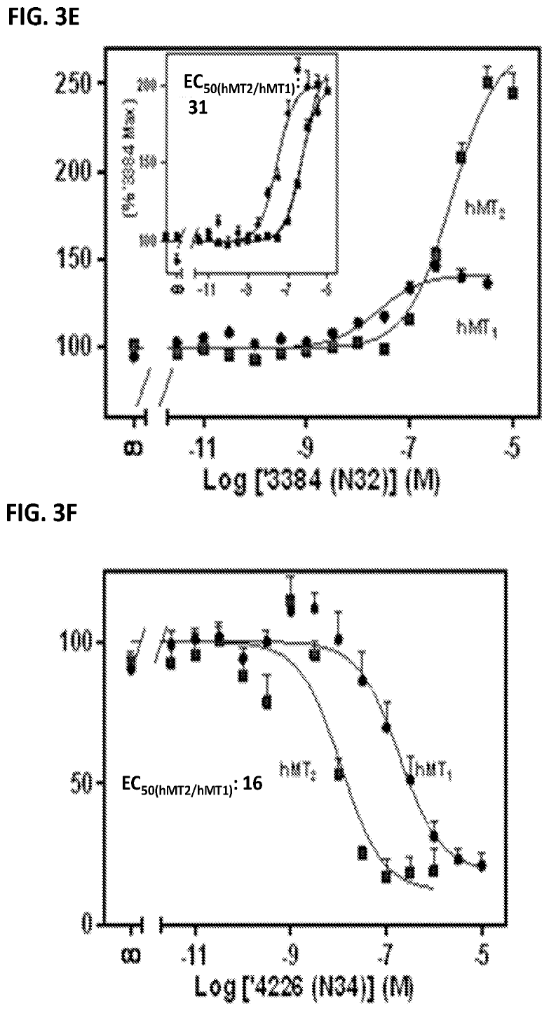

FIGS. 3A-3F. Affinity, efficacy, and potency of type selective ligands in functional assays. Competition of compounds '7447 (FIG. 3A), '3384 (FIG. 3B), and '4226 (FIG. 3C) for 2-[¹²⁵I]-iodomelatonin in stably expressed hMT₁ or hMT₂, in the presence and absence of 100 μM GTP. GTP leads to G protein uncoupling from the receptor, favoring binding of the two inverse agonists, left-shifting the binding curves towards higher potency in the presence of the nucleotide, and leading to higher selectivity between the melatonin receptor types, as expected for these inverse agonists. Data represent mean S.E.M. of five independent determinations. Concentration-response curves in transiently-expressed hMT₁ or hMT₂ receptors, monitoring isoproterenol-stimulated cAMP production for '7447 (FIG. 3D) (hMT₁ pEC₅₀: 7.39±0.10, Eₘₐₓ: −62±13% basal (n=8); hMT₂ pEC₅₀: 5.66±0.10, Eₘₐₓ: −84±9% (n=8); '3384 (FIG. 3E) hMT₁ pEC₅₀: 7.68±0.09, Eₘₐₓ: −47±12% (n=13); hMT₂ pEC₅₀: 6.18±0.04, Eₘₐₓ: −153±14% (n=13); and '4226 (FIG. 3F) hMT₁ pEC₅₀: 6.83±0.17, Eₘₐₓ: 79±3% (n=4); hMT₂ pEC₅₀: 8.15±0.09, Eₘₐₓ: 89±3% (n=4). For '7447 and '3384, data was normalized to isoproterenol-stimulated basal activity, and for '4226, data is normalized to maximal melatonin effect. Data represent mean±S.E.M of independent determinations run in triplicate. Inset graphs represent data normalized to maximal ligand effect.

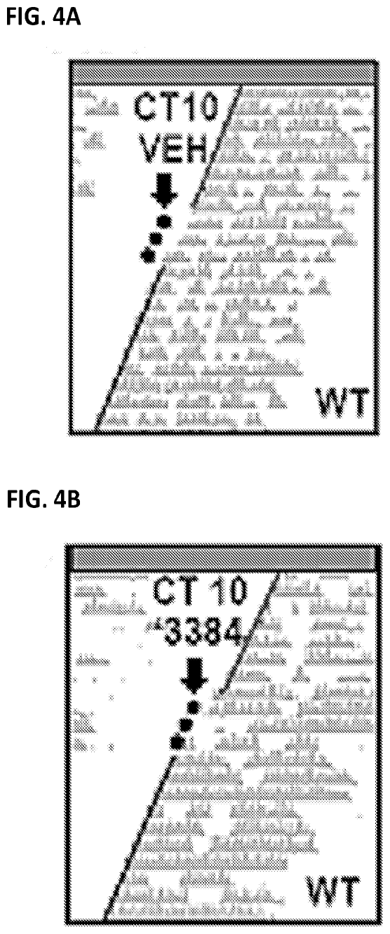

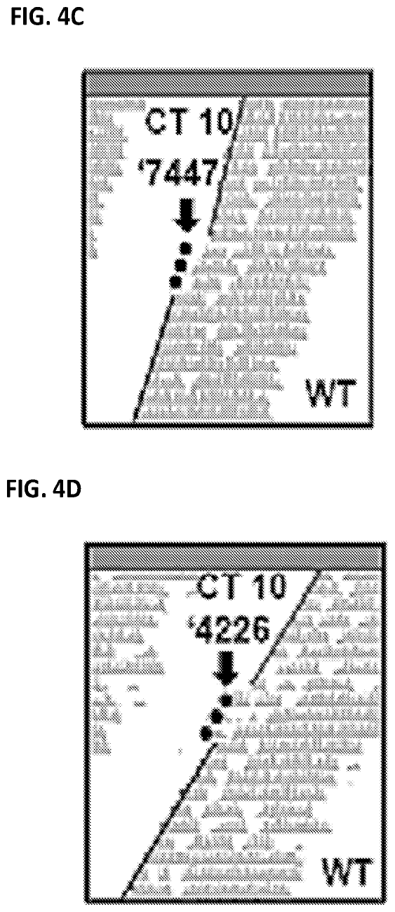

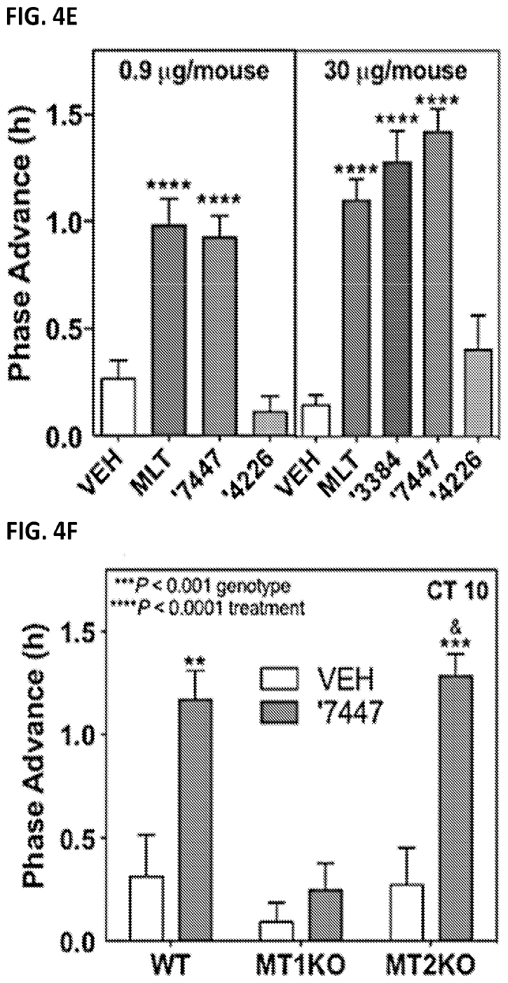

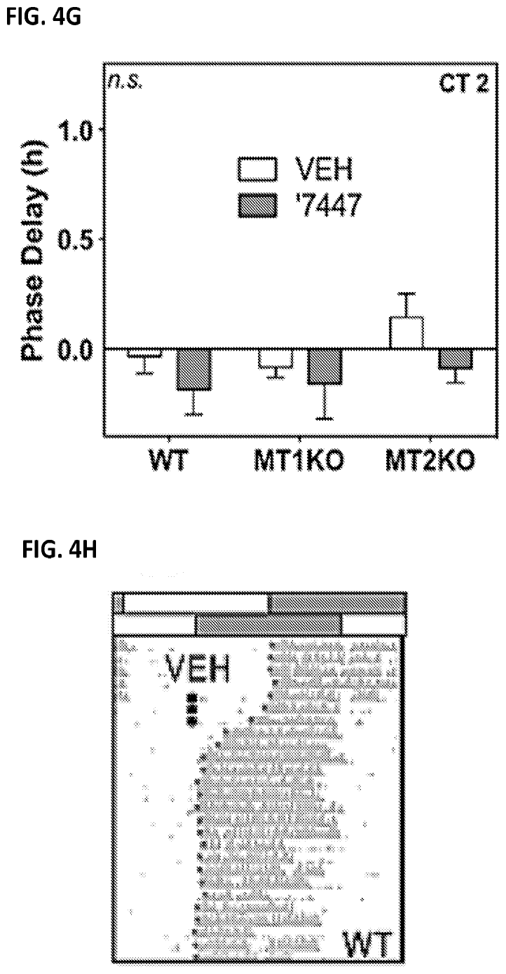

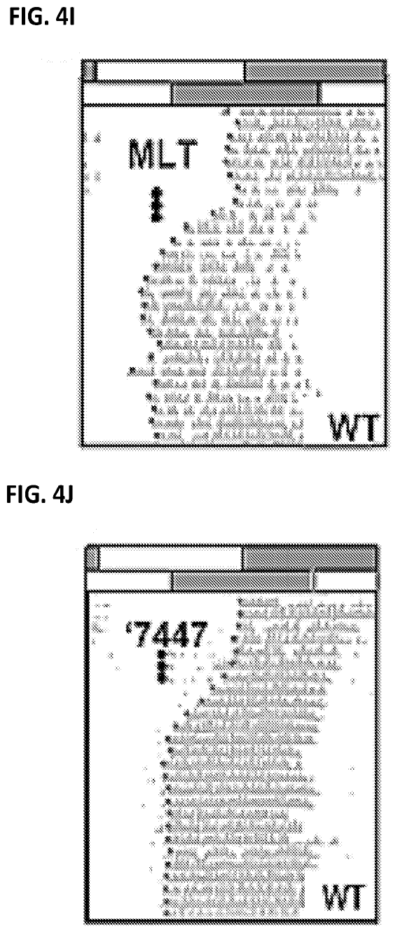

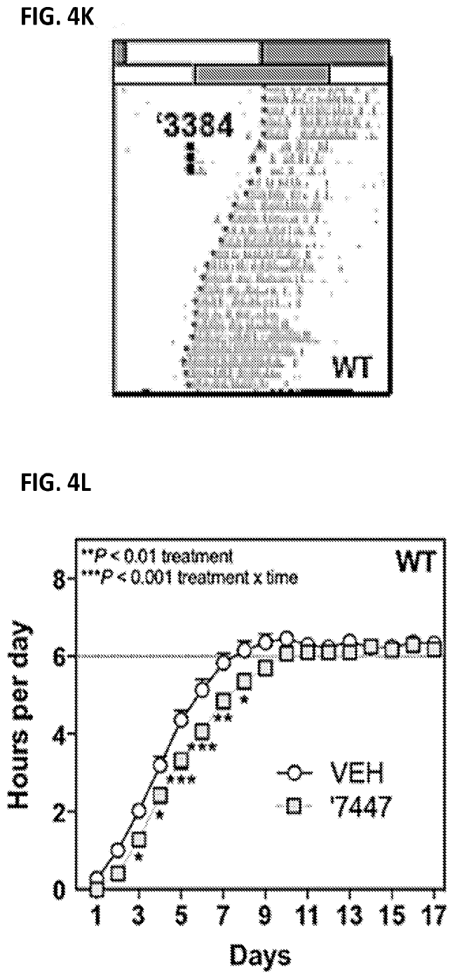

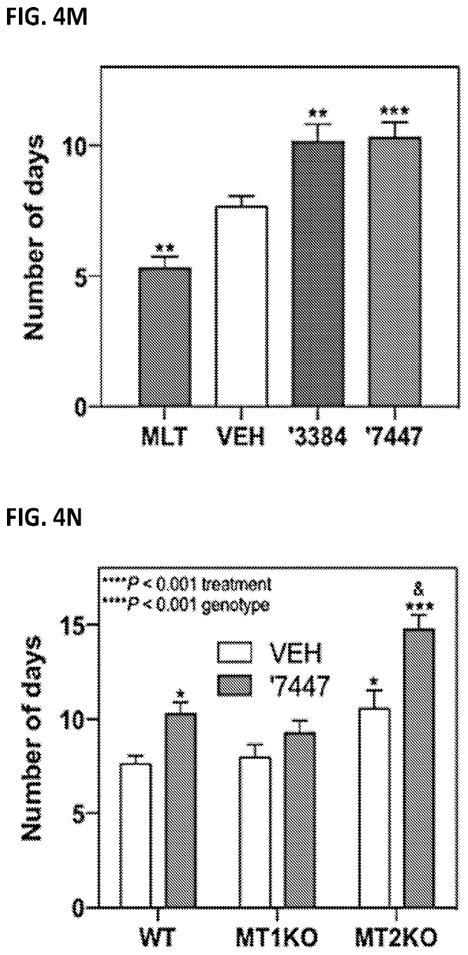

FIGS. 4A-4N. In vivo, the new MT₁-selective inverse agonists phase-advance circadian activity at dusk (CT10) and decelerate re-entrainment rate while MT₁ knockouts lose ligand sensitivity. The MT₁-selective inverse agonists '3384 and '7447 both advance circadian activity in WT mice, akin to the agonist melatonin. Meanwhile, in a jet-lag re-entrainment model, both molecules act akin to known inverse agonists. In both cases, compound activity is disrupted in MT₁KO but not MT₂KO mice. Representative of running wheel (RW) activity from individual C3H/HeN (C3H) mice kept in constant dark (gray bars) treated with vehicle (VEH) (FIG. 4A); '3384 (FIG. 4B); '7447 (FIG. 4C); and the MT₂-selective agonist '4226 (FIG. 4D) (all treatments 30 μg/mouse, s.c.). Mice were treated at circadian time 10 (CT10; 2 hours prior to onset of RW activity) for three consecutive days, shown as black dots in each actogram. Red lines indicate best-fit line of pretreatment while blue lines indicate best-fit line of post treatment onsets of RW activity that were used for phase shift determinations. (FIG. 4E) (left panel) Phase shift of RW activity onset measured in hours (h) for VEH (n=8), melatonin (MLT) (n=8), '7447 (n=13), & '4226 (n=11) treated C3H mice (0.9 μg/mouse s.c.). Comparisons made to VEH using one-way ANOVA (F_{3.36}=23.33 P=1.46×10⁻⁸) with Dunnet's post hoc test. (FIG. 4E) (right panel) Phase shift of RW activity onset measured in hours (h) for VEH (n=15), MLT (n=10), '3384 (n=16), '7447 (n=15), & '4226 (n=10) treated C3H mice (30 μg/mouse s.c.). Comparisons made to VEH using one-way ANOVA (F_{4.61}=26.45 P=9.64×10⁻¹³) with Dunnet's post hoc test. (FIG. 4F) Chemical-genetic epistasis by gene knockout supports a role for MT₁. Phase advance of RW activity onset measured in hours for VEH (white) and '7447 (blue) in C3H wild-type (WT; n=9 VEH; n=10 '7447), MT₁-knockout (MT1KO; n=8 VEH; n=8 '7447), and MT₂-knockout (MT₂KO; n=11 VEH; n=9 '7447) mice. F_{1.49}=30.59 P=1.22×10⁻⁶ for treatment, F_{2.49}=9.82 P=2.59×10⁻⁴ for genotype, and F_{2.49}=4.46 P=0.0166 for treatment x genotype interaction when compared via two-way ANOVA with Tukey's post hoc test for multiple comparisons. (FIG. 4G) Compound '7447 did not phase shift RW activity onset at CT2 (10 hours prior to RW activity onset; F_{1.49}=3.83 P=0.0564 for treatment, F_{2.49}=1.74 P=0.186 for genotype, and F_{2.49}=0.384 P=0.684 for treatment x genotype interaction when compared via two-way ANOVA with Tukey's post hoc test for multiple comparisons). VEH (white), '7447 (blue) in C3H wild-type (WT; n=8 VEH; n=8 '7447), MT₁-knockout (MT₁KO; n=6 VEH; n=7 '7447), and MT₂-knockout (MT₂KO; n=10 VEH; n=13 '7447) mice. (FIGS. 4H-4K)

Representative actograms of RW activity for VEH (h: WT), melatonin (i: WT), '7447 (j: WT), or '7447 (k: MT₂KO) treated (30 μg/mouse s.c.) mice following an advance (6 h) of the dark onset in a 12:12 light-dark cycle (gray=dark & white=light phase). Compounds were applied for 3 days 30 minutes prior to the new dark onset indicated by black dots (further details in methods).
(FIG. 4L) Rate of re-entrainment of RW activity rhythm onset in C3H WT mice expressed in hours each day advanced after a 6 hour advance of the dark phase for VEH (n=26-28 mice) vs. '7447 (n=19-21 mice). Results of a mixed-effect two-way repeated measures ANOVA revealed a significant effect of treatment (F_{1.47}=9.26 P 0.00382), time (F_{16.735}=424.4 P<1×10⁻¹⁵) as well as for treatment x time interaction (F_{16.735}=3.39 P=8.20×10⁻⁶). Tukey's post hoc test was used for multiple comparisons. (FIG. 4M) Number of days to re-entrainment of RW activity onset measured in days for VEH (n=28), MLT (n=21), '7447 (n=21), '3384 (n=16) treated C3H mice (30 μg/mouse s.c.). Comparisons with VEH made via one-way ANOVA (F_{3.82}=23.17 P=5.87×10⁻¹¹) with Dunnet's post hoc test. (FIG. 4N) Effect of genotype on number of days to re-entrainment of RW activity onset for VEH (white) vs. '7447 (blue) in C3H WT (n=28 VEH; n=21 '7447), MT₁KO (n=16 VEH; n=16 '7447), and MT₂KO (n=20 VEH; n=25 '7447) mice. F_{1.120}=24.82 P=2.14×10⁻⁶ for treatment and F_{2.120}=23.44 P=2.55×10⁻⁹ genotype when compared via two-way ANOVA with Tukey's post hoc test for multiple comparisons. Extension of FIGS. 4A-4G *P<0.05, **P<0.01, ***P<0.001, ****P<0.0001 for comparisons to WT VEH and for comparisons to MT₂KO VEH using Tukey's or Dunnet's post test (P<0.05). All bars represent mean±s.e.m. Extension of FIGS. 4H-4N.*P<0.05,**P<0.01,***P<0.001,****P<0.0001 for comparisons using Tukey's post test (P<0.05). Dotted line in 4J-4K refers to the new dark onset.

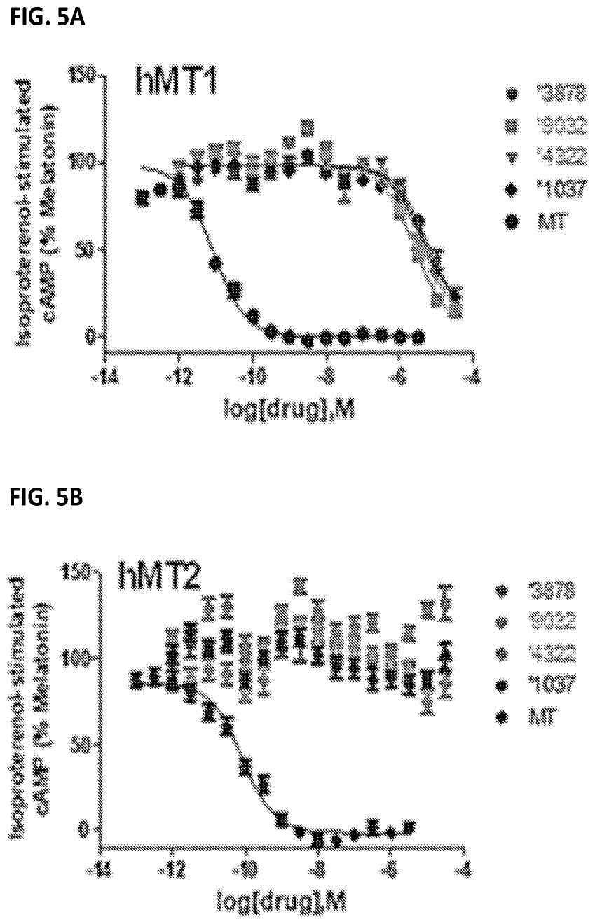

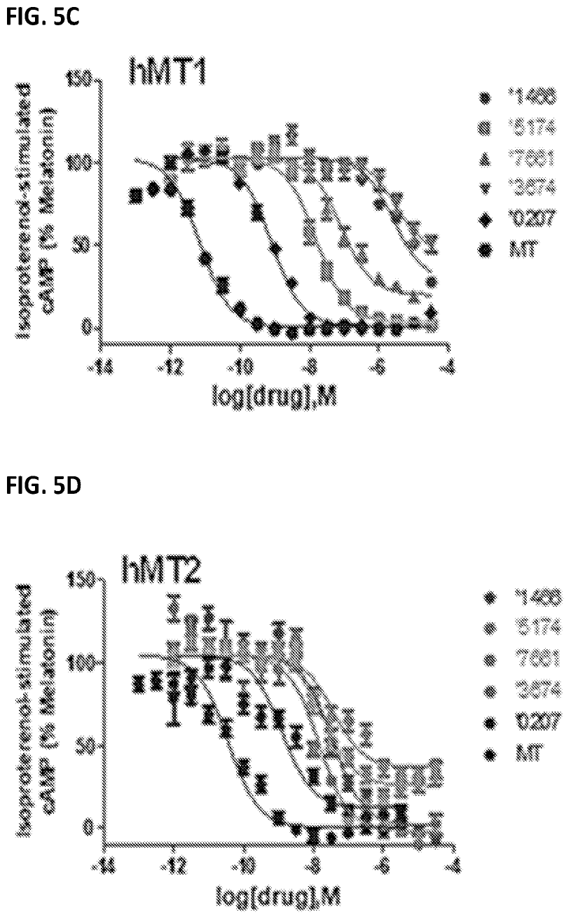

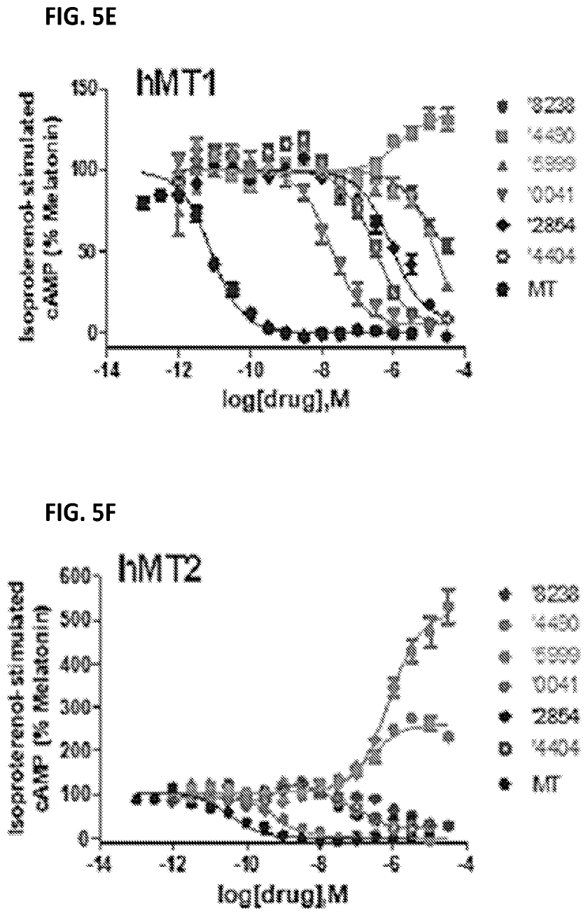

FIGS. 5A-5F. Concentration-response curves of initial 15 compounds in cAMP assays.

hMT₁- (FIGS. 5A, 5C, 5E) or hMT₂-mediated (FIGS. 5B, 5D, 5F) inhibition of isoproterenol-stimulated cAMP in HEK cells by melatonin and 15 initial compounds. Data normalized to melatonin response represent mean±S.E.M of three independent determinations run in triplicate.

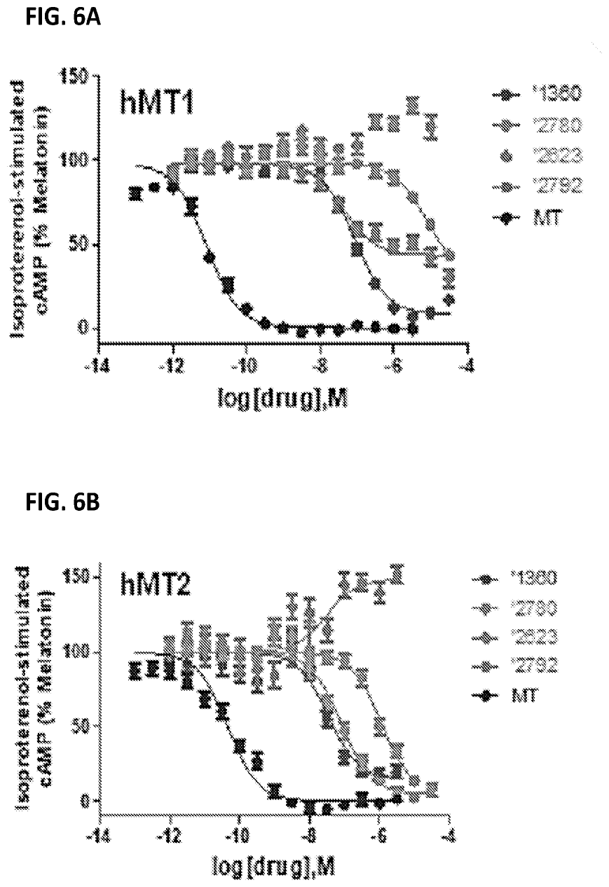

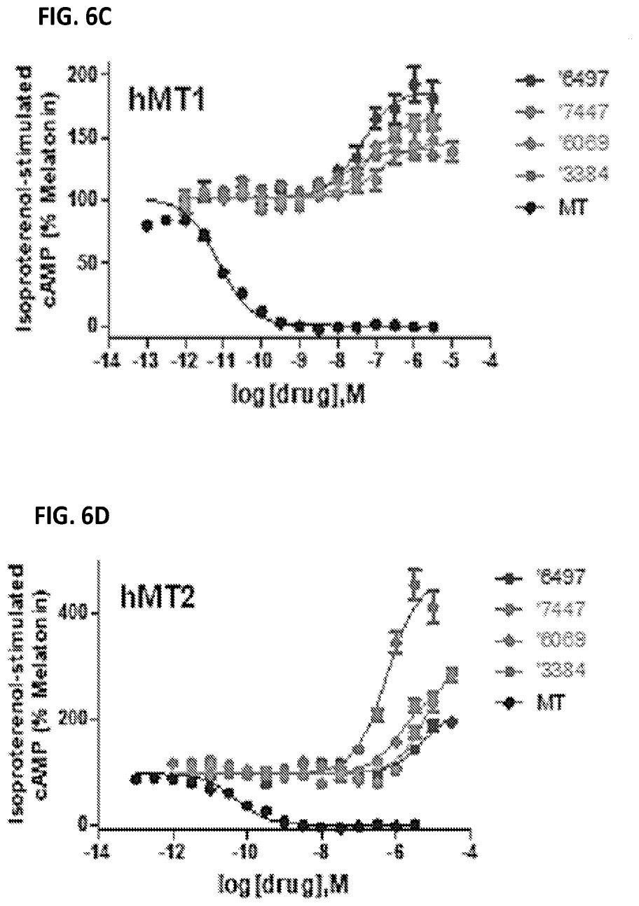

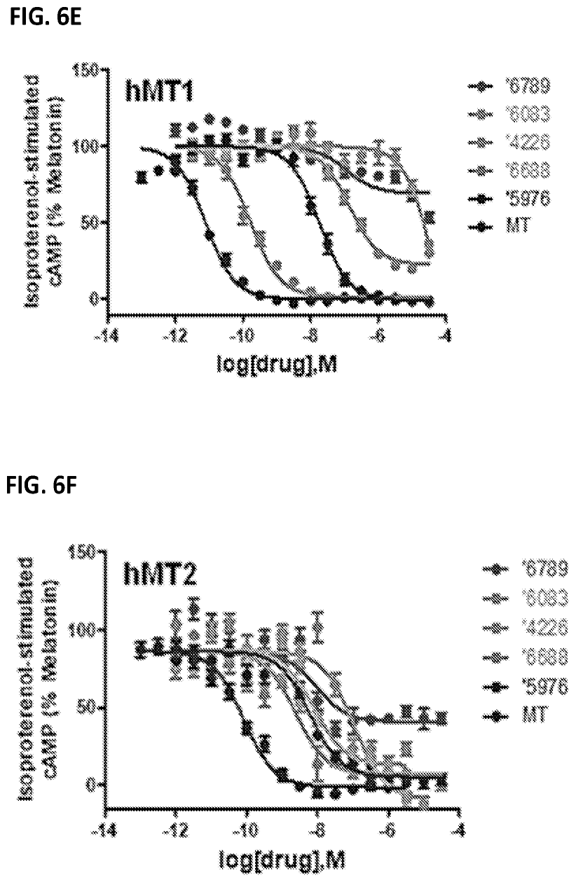

FIGS. 6A-6F. Concentration-response curves of interesting analogs based on initial hits in cAMP assays. hMT₁- (FIGS. 6A, 6C, 6E) or hMT₂-mediated (FIGS. 6B, 6D, 6F) inhibition of isoproterenol-stimulated cAMP in HEK cells by melatonin and select analogs. Data normalized to melatonin response represent mean±S.E.M of three independent determinations run in triplicate.

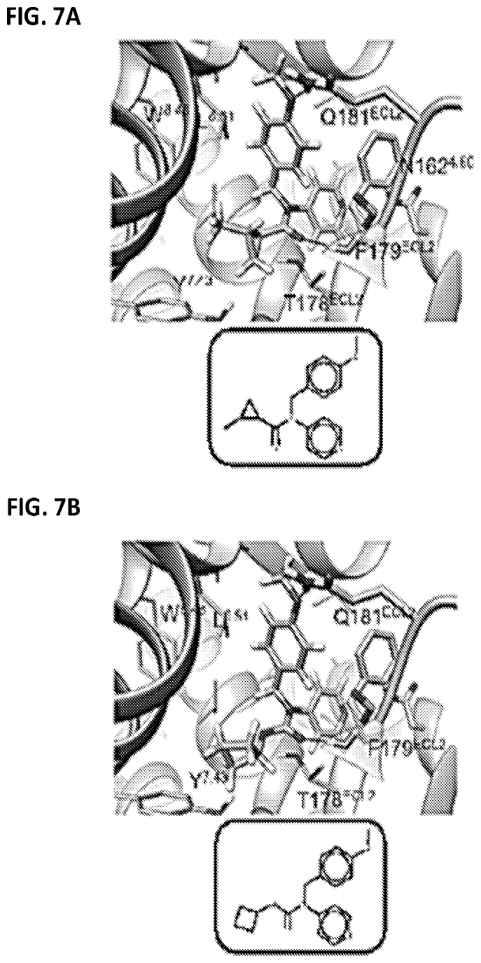

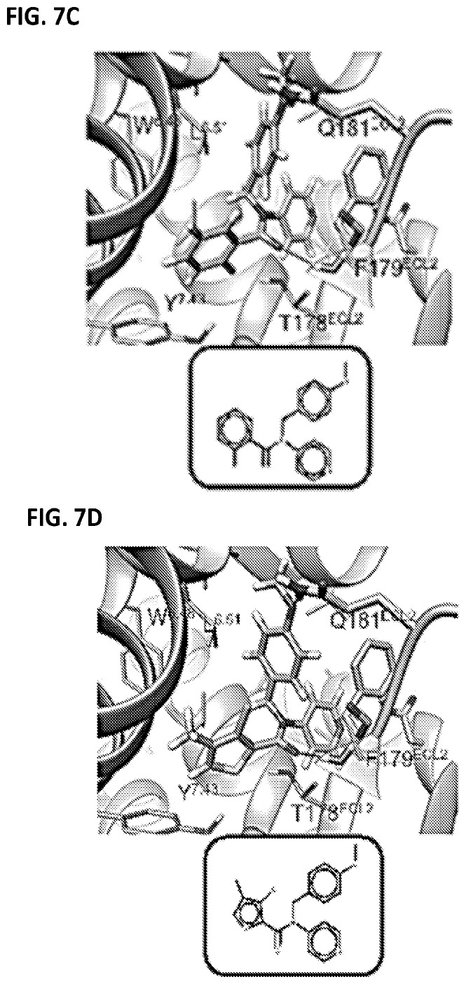

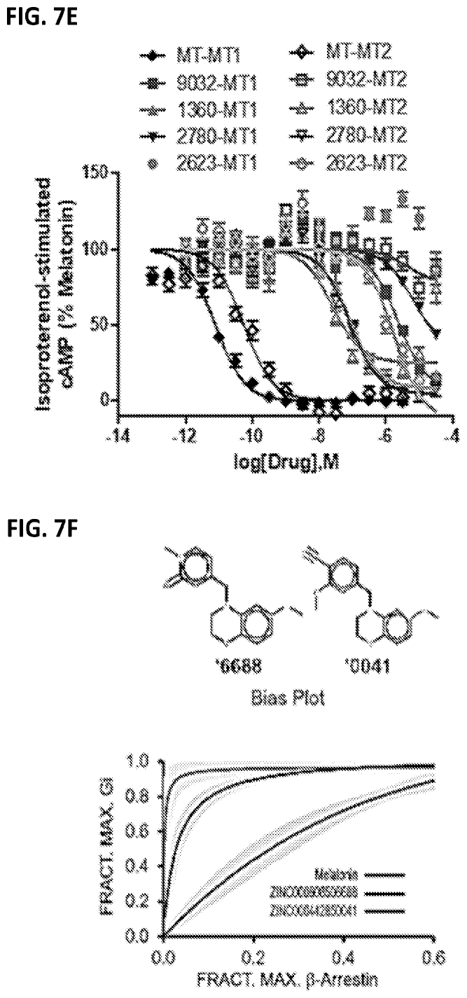

FIGS. 7A-7F. Small changes in ligand structure have large effects on melatonin receptor activity and selectivity. Docked pose of '9032, an MT₁-selective direct docking hit (FIG. 7A). Docked pose of '1360, a close analog of '9032 that switches 2-fold selectivity for MT₂ over MT₁(FIG. 7B). Docked pose of '2780, an analog where MT₂ selectivity climbs to 200-fold over MT₁ (FIG. 7C). Docked pose of '2623, which adds a bulkier 2-chloro-3-methylthiophene into a proposed MT₂-selective hydrophobic cleft, resulting in a fully MT₂-selective agonist without detectable MT₁ activity (FIG. 7D). Concentration-response curves the four analogs at MT₁ and MT₂ (FIG. 7E). Bias plots of '0041 and '6688 relative to melatonin signaling (FIG. 7F). Mean values are presented as solid lines and 95% confidence interval for the line is presented as shades. Data from minimum three independent assays.

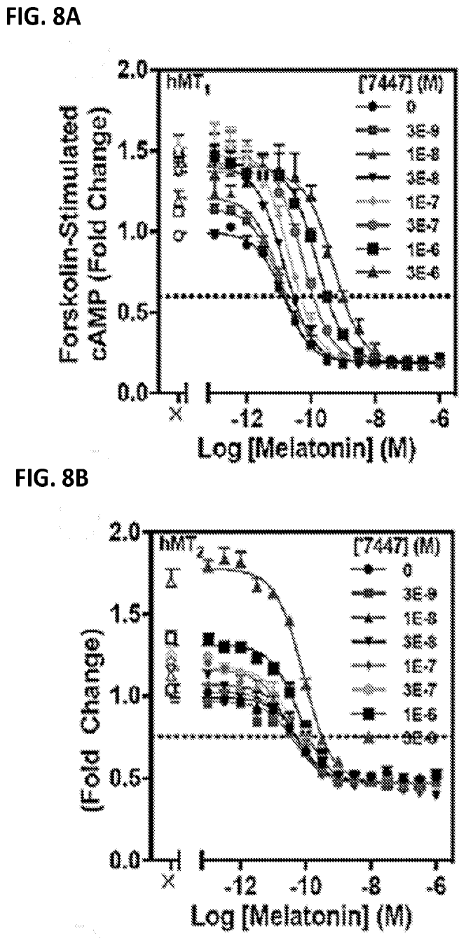

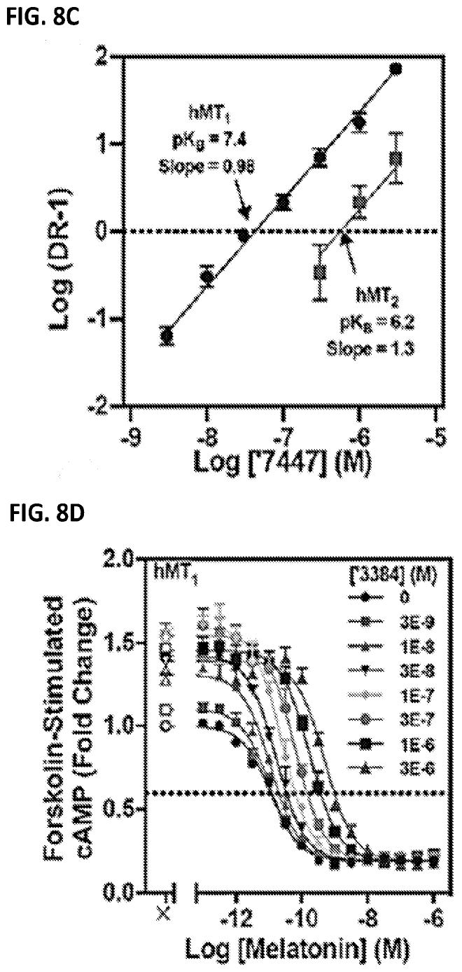

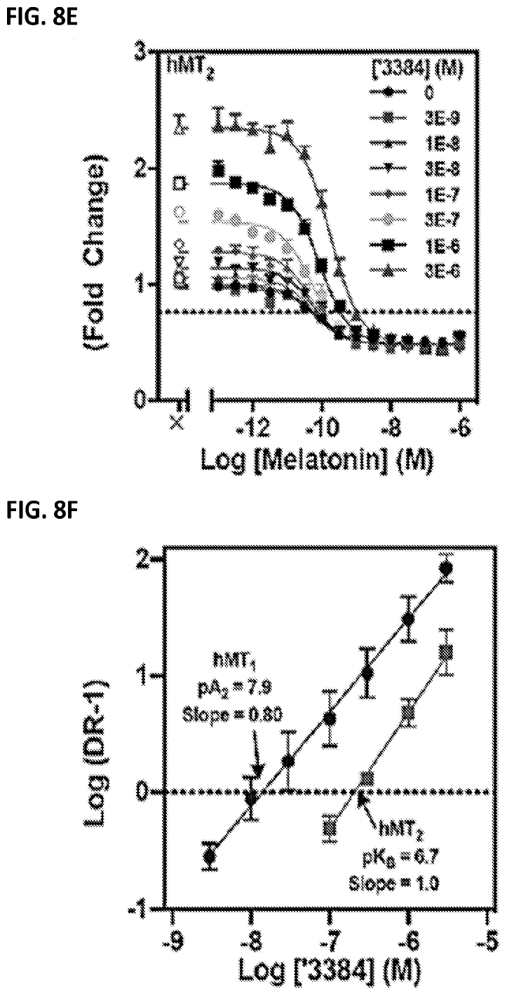

FIGS. 8A-8F. Concentration-response curves and Schild-plots of the inverse agonists '7447 and '3384 in cAMP assays. Modulation of hMT₁- (A&D) or hMT₂-mediated (B&E) inhibition of isoproterenol-stimulated cAMP in HEK cells by melatonin in the presence of '7447 (A&B) or '3384 (D&E) over a range of concentrations (FIGS. 8A-8D). Data normalized to effect of isoproterenol alone represent mean±S.E.M of three independent determinations run in triplicate. Schild plots depicting competitive antagonism of melatonin by '7447 (FIG. 8C) and '3384 (FIG. 8F. Schild analysis at hMT₁ (purple) and hMT₂ (teal) reveal competitive antagonism for '7447 (hMT₁pK_{B}: 7.4±0.1, slope: 0.98±0.03; hMT₂pK_{B}: 6.2±0.1, slope: 1.3±0.4) (FIG. 8E) and '3384 (hMT₁ pA₂: 7.9±0.1, slope: 0.80±0.04; hMT₂pK_{B}: 6.7±0.1, slope: 1.0±0.1) (FIG. 8F).

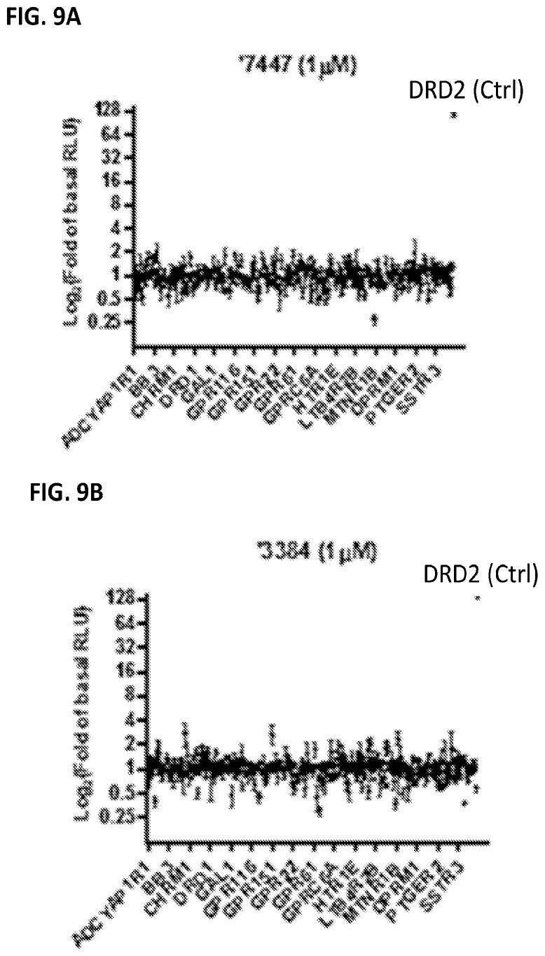

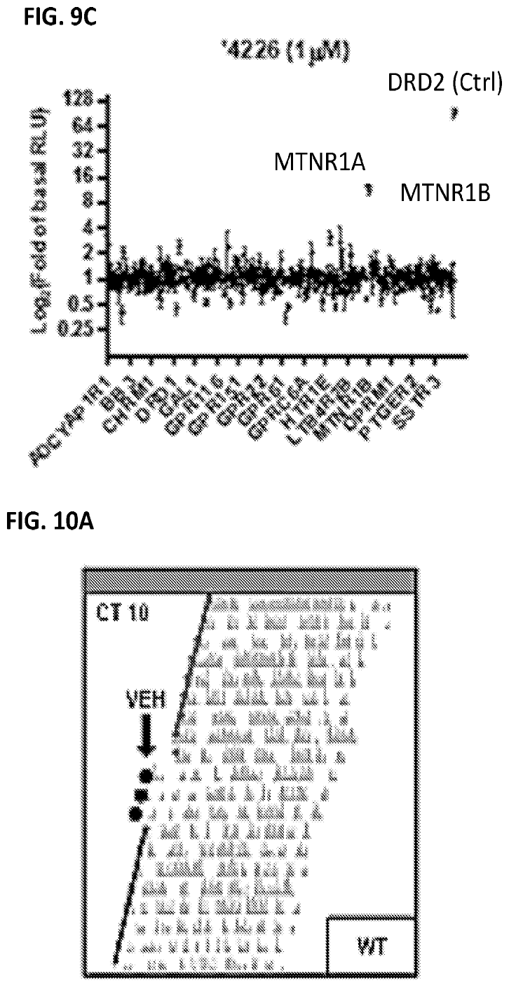

FIGS. 9A-9C. Screening of '7447, '3384 and '4226 in the PRESTO-Tango GPCR-ome. '7447 (FIG. 9A), '3384 (FIG. 9B) and '4226 (FIG. 9C) were screened against 320 non-olfactory GPCRs for agonism in the arrestin recruitment Tango assay. Each data is normalized to the basal level of luminescence and represented mean±S.E.M run in quadruplicate.

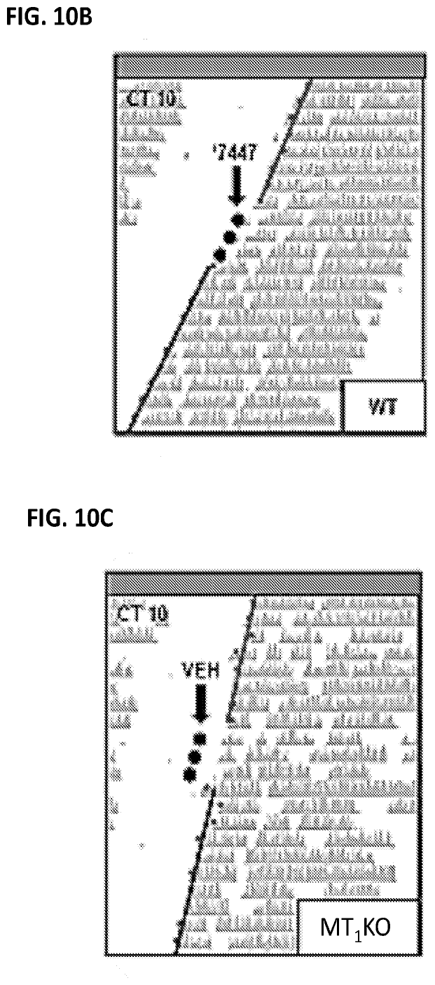

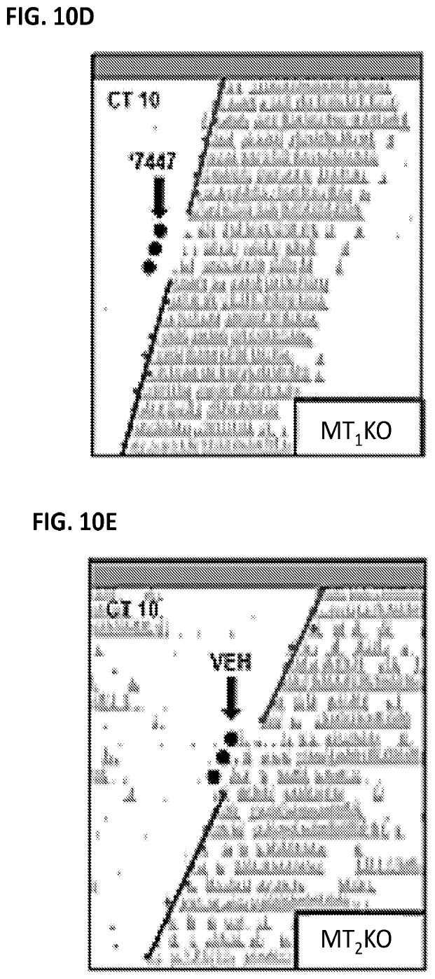

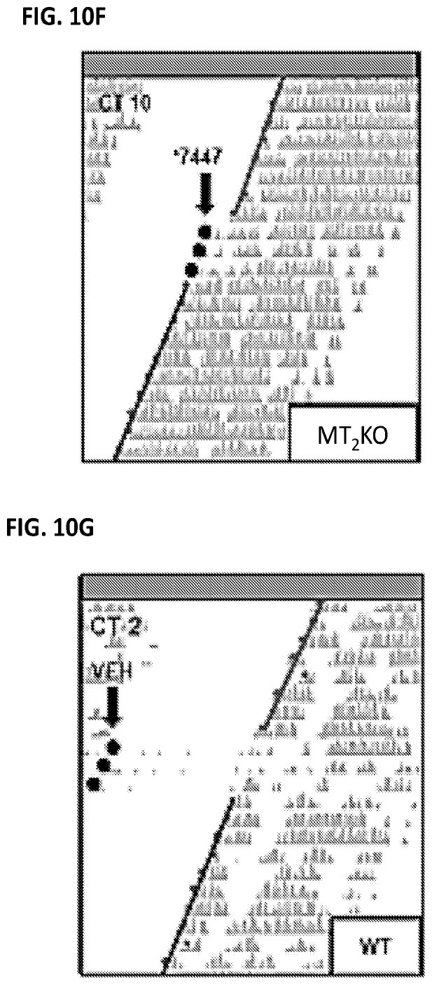

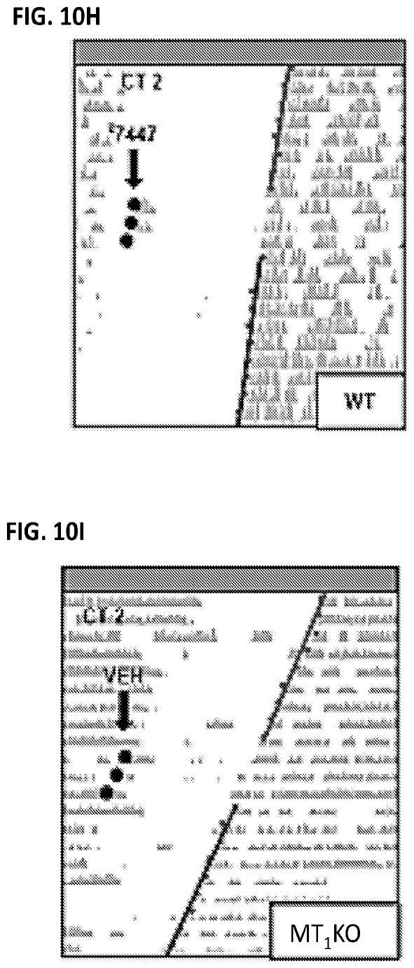

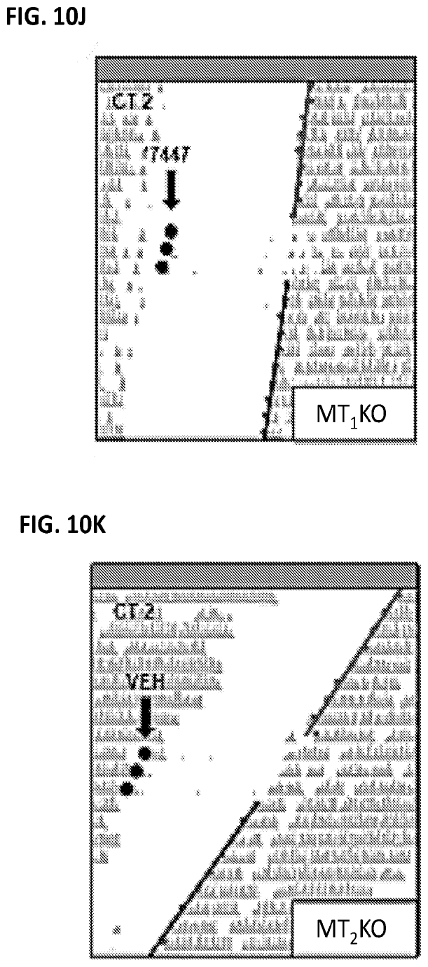

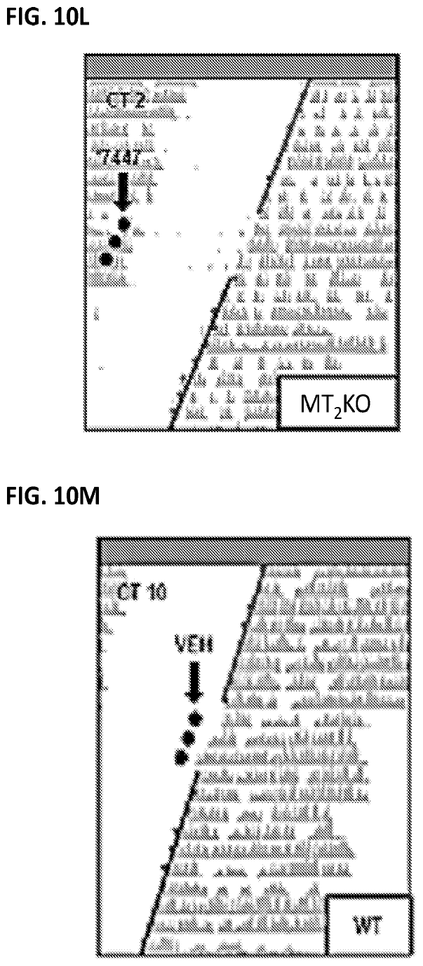

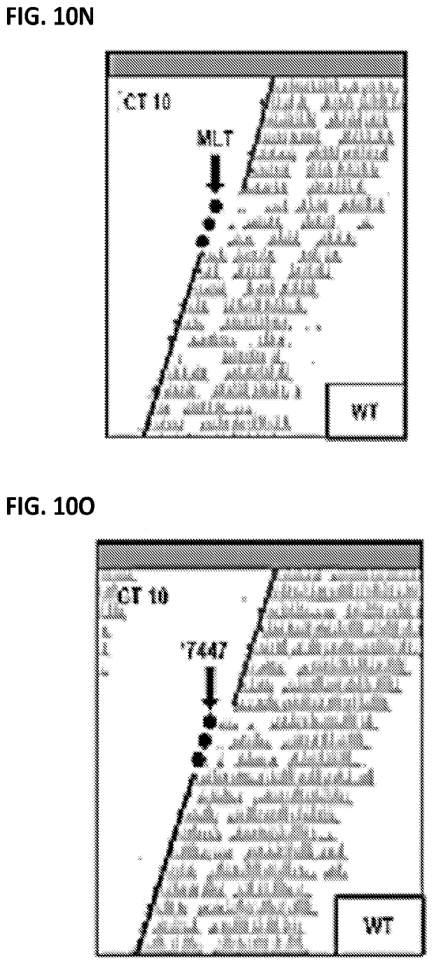

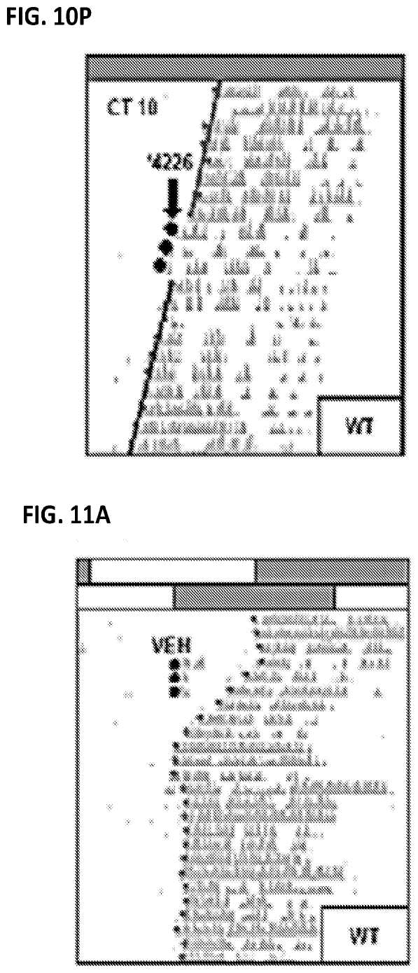

FIGS. 10A-10P. Phase shift. MT₁-selective inverse agonists phase advance circadian activity and decelerate re-entrainment rate in vivo. Representative actograms of running wheel (RW) activity from individual C3H WT (FIGS. 10A and 10B), MT₁KO (FIGS. 10C and 10D), and MT₂KO (FIGS. 10E and 10F) mice kept in constant dark treated with VEH (white; (FIGS. 10A, 10C and 10E)) or '7447 (blue; (FIGS. 10B, 10D and 10F)). Mice were treated with VEH (30% ethanol/saline s.c.) or '7447 (30 μg/mouse s.c) at circadian time 10 (CT10) for three consecutive days, shown as black dots. Red line indicates best fit line of pretreatment while blue line indicates best fit line of post treatment onsets of activity. Corresponding quantification found in FIG. 4F. Representative actograms of RW activity from individual C3H WT (FIGS. 10G and 10H), MT₁KO (FIGS. 10I and 10J), and MT₂KO (FIGS. 10K and 10L) mice kept in constant dark treated with VEH (white; (FIGS. 10G, 10I and 10K)) or '7447 (blue; (FIGS. 10H, 10J and 10L)). Mice were treated with VEH (30% ethanol/saline s.c.) or '7447 (30 μg/mouse s.c) at circadian time 2 (CT2) for three consecutive days, shown as black dots. Red line indicates best fit line of pretreatment while blue line indicates best fit line of post treatment onsets of activity. Corresponding quantification found in FIG. 4G. Representative actograms of RW activity from individual C3H/HeN (C3H) mice kept in constant dark (gray bars) treated with vehicle (VEH, 0.9% saline/30% EtOH, FIG. 10A), melatonin (MLT, FIG. 10B), '7447 (MLT, FIG. 10C), or '4226, FIG. 10D, (all treatments 0.9 μg/mouse s.c.). Mice were treated at CT10 for three consecutive days, shown as black dots in each actogram. Red lines indicate best-fit line of pretreatment while blue lines indicate best-fit line of post treatment onsets of RW activity that were used for phase shift determinations. Corresponding quantification found in FIG. 4E.

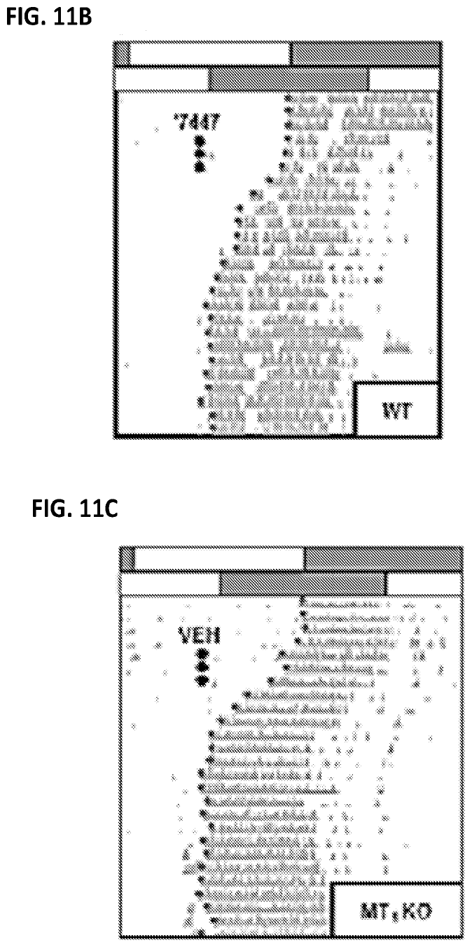

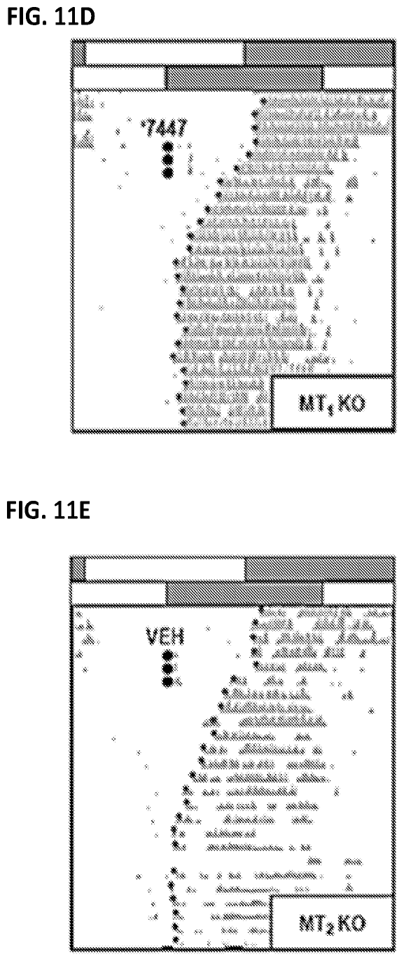

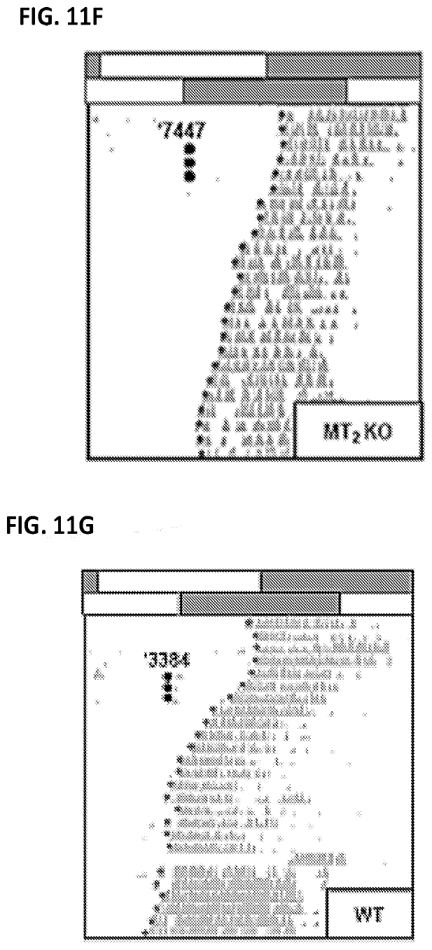

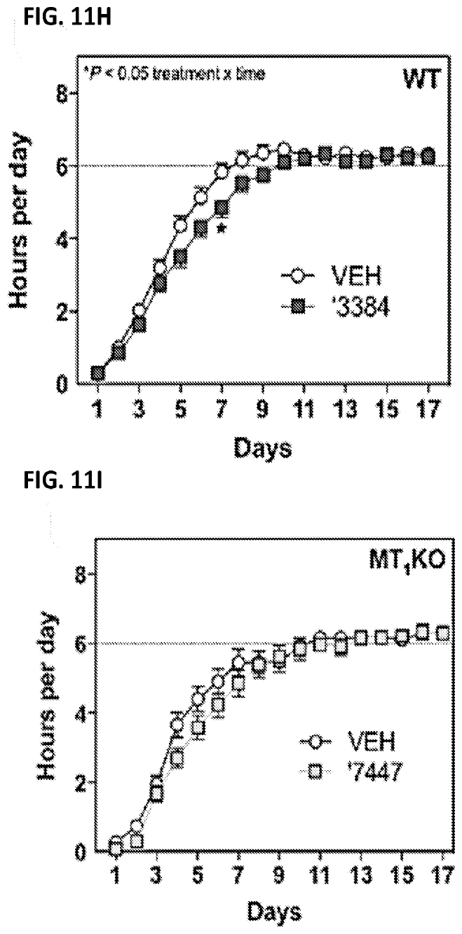

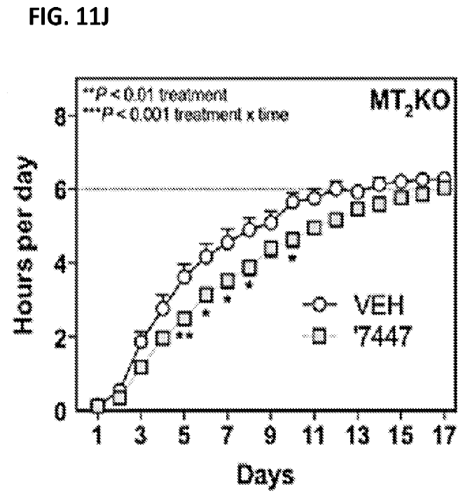

FIGS. 11A-11J. Re-entrainment. MT₁-selective inverse agonists decelerate re-entrainment rate in vivo via MT₁ receptors. Representative actograms of RW activity for VEH (FIGS. 11A, 11C and 11E): WT, MT₁KO, MT₂KO) or '7447 (FIGS. 11B, 11D and 11F: WT, MT₁KO, MT₂KO) treated C3H mice following an advance (6 hr) of the dark cycle. Mice were kept in a 12:12 light-dark cycle and compounds were applied for 3 days 30 minutes prior to the new dark onset indicated by black dots. Representative actogram of a mouse treated with 30 μg/mouse s.c. '3384 for 3 days after a 6 h shift of the LD cycle (FIG. 11G). Rate of re-entrainment of RW activity rhythm onset in C3H WT mice expressed in hours each day advanced for VEH (n=26-28 mice) vs. '3384 (n=15-16 mice). Results of a mixed-effect two-way repeated measures ANOVA revealed a significant effect of time (F_{16.647}=297.5 P<1×10⁻¹⁵) as well as for treatment x time interaction (F_{16.647}=1.99 P=0.0122), but not treatment (F_{1.52}=3.36 P=0.0726). Tukey's post hoc test was used for multiple comparisons (FIG. 11H).

Rate of re-entrainment of RW activity rhythm onset in C3H MT₁KO mice treated with VEH (n=15-16 mice) vs. '7447 (n=15-16 mice). Results of a mixed-effect two-way repeated measures ANOVA revealed a significant effect of time (F_{16.474}=227.4 P<1×10⁻¹⁵), but not treatment (F_{1.30}=1.15 P=0.292) or for treatment x time interaction (F_{16.474}=1.44 P=0.117). Tukey's post hoc test was used for multiple comparisons (FIG. 11I). Re-entrainment of RW activity rhythm onset in C3H MT₂KO mice expressed in hours each day advanced after a 6 hour advance of the dark phase for VEH (n=18-21 mice) vs. '7447 (n=25 mice). Results of a mixed-effect two-way repeated measures ANOVA revealed a significant effect of treatment (F_{1.43}=8.86 P=0.00477), time (F_{16.683}=361.0 P<1×10⁻¹⁵) as well as for treatment x time interaction (F_{16.683}=2.57 P=0.000686) (FIG. 11J).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equially encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

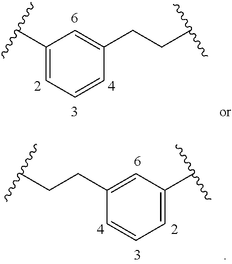 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—,

—S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
(i) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from:
(a) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. As used herein, the term “regioisomers” refers to compounds having the basic carbon skeleton unchanged but their functional groups or substituents change their position on a parent structure.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{3A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The term “receptor” as used herein refers to a protein molecule that receives chemical signals from outside a cell. When such chemical signals bind to a receptor, they cause some form of cellular/tissue response, e.g. a change in the electrical-activity of a cell.

The term “melatonin receptor” as used herein refers to G protein-coupled receptors (GPCR) which bind melatonin. Three types of melatonin receptors have been cloned. The MT₁ (or Mel_{1A} or MTNR1A) and MT2 (or Mel_{1B} or MTNR1B) receptor types are present in humans and other mammals, while an additional melatonin receptor type MT3 (or Mel_{1C} or MTNR1C) has been identified in amphibia and birds. The receptors are crucial in the signal cascade of melatonin. In the field of chronobiology, melatonin plays a key role in the synchrony of biological clocks. Melatonin secretion by the pineal gland has circadian rhythmicity regulated by the suprachiasmatic nucleus (SCN) found in the brain. The SCN functions as the timing regulator for melatonin, melatonin then follows a feedback loop to decrease SCN neuronal firing. This process is controlled by MT₁ and MT2 receptors. Melatonin receptors are found throughout the body in places such as brain, retina, cardiovascular system, liver and gallbladder, colon, skin, kidney, and others.

The term “agonist” as used herein refers to a substance which initiates a physiological response when combined with a receptor. MT₂ receptor agonist is a chemical moiety that initiates a physiological response when combined with the MT₂ receptor. MT₁ receptor agonist is a chemical moiety that initiates a physiological response when combined with the MT₁ receptor.

The term “inverse agonist” as used herein refers to a drug that binds to the same receptor as an agonist but induces a pharmacological response opposite to that of the agonist. A prerequisite for an inverse agonist response is that the receptor must have a constitutive (also known as intrinsic or basal) level activity in the absence of any ligand. In embodiments, MT₁ receptor inverse agonists bind to the MT₁ receptor and induce a pharmacological response opposite to that of the MT₁ receptor agonist.

The term “neutral antagonist” as used herein refers to an antagonist that has no activity in the absence of an agonist or inverse agonist but can block the activity of either.

The term “basal activity” as used herein refers to a signaling in the absence of an inverse agonist. The basal activity of the MT₁ receptor refers to a signaling in the absence of the MT₁ agonist.

The term “circadian rhythm” as used herein refers to any biological process that displays an endogenous, entrainable oscillation of about 24 hours and regulates periods of sleep and wakefulness. These 24-hour rhythms are driven by a circadian clock receptor. The circadian rhythm influences other biological factors such as body temperature, times for eating, and the regulation of certain hormones. These functions are calibrated by a group of cells called the suprachiasmatic nucleus (SCN) located in the hypothalamus.

The term “carcadian phase shift” as used herein refers to a shift in circadian rhythms when bedtime and wake-up time move earlier in the day (phase advance) or later in the day (phase delay).

The term “chrono molecule” as used herein refers to a chemical compound with dual or multiple efficacies during a 24 hour day. In embodiments, chrono molecules are MT₁ receptor inverse agonists. When MT₁ receptor inverse agonists are administered at dusk, these compounds do not phase delay at dawn, i.e., these compounds contribute to modulating the carcadian rhythms in a “good way”.

The term “sleep disorder or somnipathy” as used herein, is a medical disorder of the sleep patterns of a person or animal and are characterized by a difficulty falling asleep and/or staying asleep with no obvious cause.

The term “circadian rhythm sleep-wake disorders” as used herein refers to a group of diseases or conditions characterized by a disturbance or disruption to the normal circadian rhythm, which causes patients to experience excessive daytime sleepiness, insomnia, or both. This term includes delayed sleep-wake phase disorder, advanced sleep-wake phase disorder, irregular sleep-wake rhythm, non-24-hour sleep-wake rhythm disorder, shift work disorder, jet lag disorder and circadian rhythm sleep-wake disorder not otherwise specified.

The term “delayed sleep phase disorder” as used herein refers to a circadian rhythm sleep disorder in which patient's sleep pattern is delayed two hours or more from a conventional sleep pattern, causing patient to go to sleep later and wake up later.

The term “advanced sleep phase disorder” as used herein refers to a circadian rhythm sleep-wake disorder in which sleep quality and duration are normal but sleep onset and wake times are earlier than desired or earlier than socially acceptable times.

The term “jet lag” as used herein refers to a physiological condition that disrupts a person's sleep due to rapid travel across multiple time zones (usually 2 or more) and causes an imbalance to the traveler's circadian rhythm.

The term “depression” as used herein refers to condition characterized by lack of interest and pleasure in daily activities, significant weight loss or gain, insomnia or excessive sleeping, lack of energy, inability to concentrate, feelings of worthlessness or excessive guilt and recurrent thoughts of death or suicide. These symptoms could be the consequence of poor circadian rhythms regulation hence synchronizing rhythms with melatonin ligands counteracts some of the symptoms of depression.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “metabolic disorder” as used herein refers to a condition or disorder characterized by alteration of normal metabolic process by abnormal chemical reactions.

As used herein, the term “diabetes” refers to a group of metabolic discorders characterized by high blood sugar levels over a prolonged period of time. In certain instances, diabetes is represented by type 1 diabetes, type 2 diabetes, gestational diabetes, monogenic diabetes, and cystic fibrosis-related diabetes.

As used herein, the term “type 1 diabetes” refers to the condition when the body fails to produce insulin, and people with type I diabetes are insulin-dependent.

As used herein, the term “type 2 diabetes” refers to the condition when the way the body uses insulin is affected. While the body still makes insulin, the cells in the body do not respond to it as effectively. This type of diabetes is linked to obesity.

As used herein, the term “neurodegenerative disorder” refers to a condition that is characterized by progressive loss of structure or function of neurons, including neurons' death. Examples of neurodegenerative disorders include, but are not limited, to amyotrophic lateral sclerosis, Parkinson's disease, Alzheimer's disease, Huntington's disease, which are incurable, resulting in progressive degeneration and/or death of neuron cells. Neurodegeneration can be found in many different levels of neuronal circuitry ranging from molecular to systemic.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the term “co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the terms “selective” or “selectivity” or the like of a compound refers to the compound's ability to discriminate between molecular targets.

As used herein, the terms “specific”, “specifically”, “specificity”, or the like of a compound refers to the compound's ability to cause a particular action, such as inhibition, to a particular molecular target with minimal or no action to other proteins in the cell.

Compounds

In an aspect, provided herein is a compound having the formula (I):

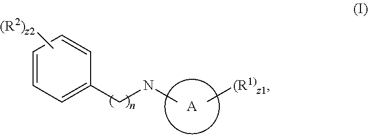

or a pharmaceutically acceptable salt thereof.

n is and integer from 0 to 5. z1 is an integer from 0 to 2. z2 is an integer from 0 to 5. Ring A is a substituted or unsubstituted aryl or substituted or unsubstituted heteroaryl. R¹ is independently halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl, substituted or unsubstituted aryl, or substituted or unsubstituted heteroaryl. R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl, substituted or unsubstituted aryl, or substituted or unsubstituted heteroaryl. X¹ and X² are independently halogen.

In embodiments, Ring A is independently, substituted (e.g., substituted with a substituent group, a size-limited substituent group, or lower substituent group) or unsubstituted aryl (e.g., C₆-C₁₀ aryl, C₁₀ aryl, or phenyl) or substituted (e.g., substituted with a substituent group, a size-limited substituent group, or lower substituent group) or unsubstituted heteroaryl (e.g., 5 to 10 membered heteroaryl, 5 to 9 membered heteroaryl, or 5 to 6 membered heteroaryl). In embodiments, Ring A is a substituted or unsubstituted (C₆-C₁₀) aryl, or substituted or unsubstituted 5 to 10 membered heteroaryl.

In embodiments, ring A is a 6-membered substituted or unsubstituted aryl or a 6-membered substituted or unsubstituted heteroaryl.

In embodiments, Ring A is R¹-substituted or unsubstituted phenyl, R¹-substituted or unsubstituted pyridinyl, or R¹-substituted or unsubstituted pyrrolo[3,2-b]pyridinyl. In embodiments, Ring A is unsubstituted phenyl. In embodiments, Ring A is unsubstituted pyridinyl. In embodiments, Ring A is unsubstituted pyrrolo[3,2-b]pyridinyl.

In embodiments, ring A may be substituted with one R¹ substituent. In embodiments, ring A may be substituted with two optionally different R¹ substituents. In embodiments, ring A may be unsubstituted.

In embodiments, the compound has the formula (Ia) or (Ic).

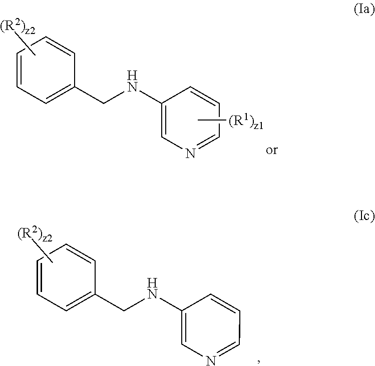

pharmaceutically acceptable salt thereof, wherein R¹, R², z1 and z2 are as defined above, including embodiments thereof.

In embodiments, phenyl ring may be substituted with one R² substituent. In embodiments, phenyl ring may be substituted with two optionally different R² substituents. In embodiments, phenyl ring may be substituted with three optionally different R² substituents. In embodiments, phenyl ring may be substituted with four optionally different R² substituents. In embodiments, phenyl ring may be substituted with five optionally different R² substituents. In embodiments, phenyl ring may be unsubstituted.

R¹ is independently halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is halogen (e.g., —F, —Cl, Br, —I), —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{A1}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, (e.g., —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X¹ is independently —F, —Cl, —Br, or —I.

In embodiments, R¹ is —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R¹ is —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R¹ is

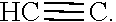

In embodiments, each R^{1A} and R^{1B} are independently hydrogen, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —COOH, —CONH₂, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₁₀, or phenyl), substituted (e.g., substituted with a substituent group, a size-limited substituent group, or lower substituent group) or unsubstituted heteroaryl (e.g., 5 to 10 membered, 5 to 9 membered, or 5 to 6 membered), or R^{1A} and R^{1B} substituents bonded to the same nitrogen atom may optionally be joined to form a substituted or unsubstituted heterocycloalkyl, substituted or unsubstituted aryl, or substituted or unsubstituted heteroaryl.

In embodiments, each R^{1A} and R^{1B} are independently hydrogen, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —COOH, —CONH₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —N^{2A}SO₂R^{2B}—NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁴-substituted or unsubstituted alkyl (e.g., C₁-C₈ alkyl, C₁-C₆ alkyl, or C₁-C₄ alkyl), R⁴-substituted or unsubstituted heteroalkyl (e.g., 2 to 8 membered heteroalkyl, 2 to 6 membered heteroalkyl, or 2 to 4 membered heteroalkyl), R⁴-substituted or unsubstituted cycloalkyl (e.g., C₃-C₈ cycloalkyl, C₃-C₆ cycloalkyl, or C₅-C₆ cycloalkyl), R⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴-substituted or unsubstituted aryl (e.g., C₆-C₁₀ aryl, C₁₀ aryl, or phenyl), or R⁴-substituted or unsubstituted heteroaryl (e.g., 5 to 10 membered heteroaryl, 5 to 9 membered heteroaryl, or 5 to 6 membered heteroaryl).

In embodiments, R² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R² is R⁴-substituted or unsubstituted alkyl (e.g., C₁-C₈ alkyl, C₁-C₆ alkyl, or C₁-C₄ alkyl). In embodiments, R² is R⁴-substituted or unsubstituted heteroalkyl (e.g., 2 to 8 membered heteroalkyl, 2 to 6 membered heteroalkyl, or 2 to 4 membered heteroalkyl). In embodiments, R² is R⁴-substituted or unsubstituted cycloalkyl (e.g., C₃-C₈ cycloalkyl, C₃-C₆ cycloalkyl, or C₅-C₆ cycloalkyl 1). In embodiments, R² is R⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R² is R⁴-substituted or unsubstituted aryl (e.g., C₆-C₁₀ aryl, C₁₀ aryl, or phenyl). In embodiments, R² is R⁴-substituted or unsubstituted heteroaryl (e.g., 5 to 10 membered heteroaryl, 5 to 9 membered heteroaryl, or 5 to 6 membered heteroaryl).

R⁴ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁵-substituted or unsubstituted alkyl (e.g., C₁-C₈ alkyl, C₁-C₆ alkyl, or C₁-C₄ alkyl), R⁵-substituted or unsubstituted heteroalkyl (e.g., 2 to 8 membered heteroalkyl, 2 to 6 membered heteroalkyl, or 2 to 4 membered heteroalkyl), R⁵-substituted or unsubstituted cycloalkyl (e.g., C₃-C₈ cycloalkyl, C₃-C₆ cycloalkyl, or C₅-C₆ cycloalkyl), R⁵-substituted or unsubstituted heterocycloalkyl (e.g., 3 to 8 membered heterocycloalkyl, 3 to 6 membered heterocycloalkyl, or 5 to 6 membered heterocycloalkyl), R⁵-substituted or unsubstituted aryl (e.g., C₆-C₁₀ aryl, C₁₀ aryl, or phenyl), or R⁵-substituted or unsubstituted heteroaryl (e.g., 5 to 10 membered heteroaryl, 5 to 9 membered heteroaryl, or 5 to 6 membered heteroaryl).

In embodiments, R⁴ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁴ is R⁵-substituted or unsubstituted alkyl (e.g., C₁-C₈ alkyl, C₁-C₆ alkyl, or C₁-C₄ alkyl). In embodiments, R⁴ is R⁵-substituted or unsubstituted heteroalkyl (e.g., 2 to 8 membered heteroalkyl, 2 to 6 membered heteroalkyl, or 2 to 4 membered heteroalkyl). In embodiments, R⁴ is R⁵-substituted or unsubstituted cycloalkyl (e.g., C₃-C₈ cycloalkyl, C₃-C₆ cycloalkyl, or C₅-C₆ cycloalkyl). In embodiments, R⁴ is R⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is R⁵-substituted or unsubstituted aryl (e.g., C₆-C₁₀ aryl, C₁₀ aryl, or phenyl). In embodiments, R⁴ is R⁵-substituted or unsubstituted heteroaryl (e.g., 5 to 10 membered heteroaryl, 5 to 9 membered heteroaryl, or 5 to 6 membered heteroaryl).

R⁵ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁶-substituted or unsubstituted alkyl (e.g., C₁-C₈ alkyl, C₁-C₆ alkyl, or C₁-C₄ alkyl), R⁶-substituted or unsubstituted heteroalkyl (e.g., 2 to 8 membered heteroalkyl, 2 to 6 membered heteroalkyl, or 2 to 4 membered heteroalkyl), R⁶-substituted or unsubstituted cycloalkyl (e.g., C₃-C₈ cycloalkyl, C₃-C₆ cycloalkyl, or C₅-C₆ cycloalkyl), R⁶-substituted or unsubstituted heterocycloalkyl (e.g., 3 to 8 membered heterocycloalkyl, 3 to 6 membered heterocycloalkyl, or 5 to 6 membered heterocycloalkyl), R⁶-substituted or unsubstituted aryl (e.g., C₆-C₁₀ aryl, C₁₀ aryl, or phenyl), or R⁶-substituted or unsubstituted heteroaryl (e.g., 5 to 10 membered heteroaryl, 5 to 9 membered heteroaryl, or 5 to 6 membered heteroaryl).

In embodiments, R⁵ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁵ is R⁶-substituted or unsubstituted alkyl (e.g., C₁-C₈ alkyl, C₁-C₆ alkyl, or C₁-C₄ alkyl). In embodiments, R⁵ is R⁶-substituted or unsubstituted heteroalkyl (e.g., 2 to 8 membered heteroalkyl, 2 to 6 membered heteroalkyl, or 2 to 4 membered heteroalkyl). In embodiments, R⁵ is R⁶-substituted or unsubstituted cycloalkyl (e.g., C₃-C₈ cycloalkyl, C₃-C₆ cycloalkyl, or C₅-C₆ cycloalkyl). In embodiments, R⁵ is R⁶-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R⁶-substituted or unsubstituted aryl (e.g., C₆-C₁₀ aryl, C₁₀ aryl, or phenyl). In embodiments, R⁵ is R⁶-substituted or unsubstituted heteroaryl (e.g., 5 to 10 membered heteroaryl, 5 to 9 membered heteroaryl, or 5 to 6 membered heteroaryl).

R⁶ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁶ is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁶ is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R² is halogen, OR^{2A}, substituted or unsubstituted alkyl, substituted or unsubstituted cycloalkyl, —CN or R^{2A} and R^{2B} are joined together to form a substituted or unsubstituted heteroaryl. In embodiments, R² is halogen. In embodiments, R² is —F, —Cl, —Br, or —I. In embodiments, R² is OR^{2A}. In embodiments, R^{2A} is substituted or unsubstituted alkyl. In embodiments, R^{2A} is substituted or unsubstituted C₁-C₃ alkyl. In embodiments, R² is substituted or unsubstituted alkyl. In embodiments, R² is substituted or unsubstituted C₁-C₄ alkyl. In embodiments, R² is unsubstituted C₁-C₂ alkyl. In embodiments, R² is substituted C₃-C₄ alkyl. In embodiments, R² is unsubstituted C₃-C₈ cycloalkyl. In embodiments, R² is unsubstituted cyclopropyl. In embodiments, R² is unsubstituted cyclobutyl. In embodiments, R² is —CN. In embodiments, R² is unsubstituted heteroaryl. In embodiments, R² is benzo[d][1,3]dioxole.

In embodiments, R^{2A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4A}-substituted or unsubstituted alkyl (e.g., C₁-C₈ alkyl, C₁-C₆ alkyl, or C₁-C₄ alkyl), R^{4A}-substituted or unsubstituted heteroalkyl (e.g., 2 to 8 membered heteroalkyl, 2 to 6 membered heteroalkyl, or 2 to 4 membered heteroalkyl), R^{4A}-substituted or unsubstituted cycloalkyl (e.g., C₃-C₈ cycloalkyl, C₃-C₆ cycloalkyl, or C₅-C₆ cycloalkyl), R^{4A}-substituted or unsubstituted heterocycloalkyl (e.g., 3 to 8 membered heterocycloalkyl, 3 to 6 membered heterocycloalkyl, or 5 to 6 membered heterocycloalkyl), R^{4A}-substituted or unsubstituted aryl (e.g., C₆-C₁₀ aryl, C₁₀ aryl, or phenyl), or R^{4A}-substituted or unsubstituted heteroaryl (e.g., 5 to 10 membered heteroaryl, 5 to 9 membered heteroaryl, or 5 to 6 membered heteroaryl).

In embodiments, R^{2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2A} is R^{4A}-substituted or unsubstituted alkyl (e.g., C₁-C₈ alkyl, C₁-C₆ alkyl, or C₁-C₄ alkyl). In embodiments, R^{2A} is R^{4A}-substituted or unsubstituted heteroalkyl (e.g., 2 to 8 membered heteroalkyl, 2 to 6 membered heteroalkyl, or 2 to 4 membered heteroalkyl). In embodiments, R^{2A} is R^{4A}-substituted or unsubstituted cycloalkyl (e.g., C₃-C₈ cycloalkyl, C₃-C₆ cycloalkyl, or C₅-C₆ cycloalkyl 1). In embodiments, R^{2A} is R^{4A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is R^{4A}-substituted or unsubstituted aryl (e.g., C₆-C₁₀ aryl, C₁₀ aryl, or phenyl). In embodiments, R^{2A} is R^{4A}-substituted or unsubstituted heteroaryl (e.g., 5 to 10 membered heteroaryl, 5 to 9 membered heteroaryl, or 5 to 6 membered heteroaryl).

R^{4A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5A}-substituted or unsubstituted alkyl (e.g., C₁-C₈ alkyl, C₁-C₆ alkyl, or C₁-C₄ alkyl), R^{5A}-substituted or unsubstituted heteroalkyl (e.g., 2 to 8 membered heteroalkyl, 2 to 6 membered heteroalkyl, or 2 to 4 membered heteroalkyl), R^{5A}-substituted or unsubstituted cycloalkyl (e.g., C₃-C₈ cycloalkyl, C₃-C₆ cycloalkyl, or C₅-C₆ cycloalkyl), R^{5A}-substituted or unsubstituted heterocycloalkyl (e.g., 3 to 8 membered heterocycloalkyl, 3 to 6 membered heterocycloalkyl, or 5 to 6 membered heterocycloalkyl), R^{5A}-substituted or unsubstituted aryl (e.g., C₆-C₁₀ aryl, C₁₀ aryl, or phenyl), or R^{5A}-substituted or unsubstituted heteroaryl (e.g., 5 to 10 membered heteroaryl, 5 to 9 membered heteroaryl, or 5 to 6 membered heteroaryl).

In embodiments, R^{4A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4A} is R^{5A}-substituted or unsubstituted alkyl (e.g., C₁-C₈ alkyl, C₁-C₆ alkyl, or C₁-C₄ alkyl). In embodiments, R^{4A} is R^{5A}-substituted or unsubstituted heteroalkyl (e.g., 2 to 8 membered heteroalkyl, 2 to 6 membered heteroalkyl, or 2 to 4 membered heteroalkyl). In embodiments, R^{4A} is R^{5A}-substituted or unsubstituted cycloalkyl (e.g., C₃-C₈ cycloalkyl, C₃-C₆ cycloalkyl, or C₅-C₆ cycloalkyl). In embodiments, R^{4A} is R^{5A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4A} is R^{5A}-substituted or unsubstituted aryl (e.g., C₆-C₁₀ aryl, C₁₀ aryl, or phenyl). In embodiments, R^{4A} is R^{5A}-substituted or unsubstituted heteroaryl (e.g., 5 to 10 membered heteroaryl, 5 to 9 membered heteroaryl, or 5 to 6 membered heteroaryl).

R^{5A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6A}-substituted or unsubstituted alkyl (e.g., C₁-C₈ alkyl, C₁-C₆ alkyl, or C₁-C₄ alkyl), R^{6A}-substituted or unsubstituted heteroalkyl (e.g., 2 to 8 membered heteroalkyl, 2 to 6 membered heteroalkyl, or 2 to 4 membered heteroalkyl), R^{6A}-substituted or unsubstituted cycloalkyl (e.g., C₃-C₈ cycloalkyl, C₃-C₆ cycloalkyl, or C₅-C₆ cycloalkyl), R^{6A}-substituted or unsubstituted heterocycloalkyl (e.g., 3 to 8 membered heterocycloalkyl, 3 to 6 membered heterocycloalkyl, or 5 to 6 membered heterocycloalkyl), R^{6A}-substituted or unsubstituted aryl (e.g., C₆-C₁₀ aryl, C₁₀ aryl, or phenyl), or R^{6A}-substituted or unsubstituted heteroaryl (e.g., 5 to 10 membered heteroaryl, 5 to 9 membered heteroaryl, or 5 to 6 membered heteroaryl).

In embodiments, R^{5A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5A} is R^{6A}-substituted or unsubstituted alkyl (e.g., C₁-C₈ alkyl, C₁-C₆ alkyl, or C₁-C₄ alkyl). In embodiments, R^{5A} is R^{6A}-substituted or unsubstituted heteroalkyl (e.g., 2 to 8 membered heteroalkyl, 2 to 6 membered heteroalkyl, or 2 to 4 membered heteroalkyl). In embodiments, R^{5A} is R^{6A}-substituted or unsubstituted cycloalkyl (e.g., C₃-C₈ cycloalkyl, C₃-C₆ cycloalkyl, or C₅-C₆ cycloalkyl). In embodiments, R^{5A} is R^{6A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5A} is R^{6A}-substituted or unsubstituted aryl (e.g., C₆-C₁₀ aryl, C₁₀ aryl, or phenyl). In embodiments, R^{5A} is R^{6A}-substituted or unsubstituted heteroaryl (e.g., 5 to 10 membered heteroaryl, 5 to 9 membered heteroaryl, or 5 to 6 membered heteroaryl).

R^{6A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6A} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2B} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2B} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4B}-substituted or unsubstituted alkyl (e.g., C₁-C₈ alkyl, C₁-C₆ alkyl, or C₁-C₄ alkyl), R^{4B}-substituted or unsubstituted heteroalkyl (e.g., 2 to 8 membered heteroalkyl, 2 to 6 membered heteroalkyl, or 2 to 4 membered heteroalkyl), R^{4B}-substituted or unsubstituted cycloalkyl (e.g., C₃-C₈ cycloalkyl, C₃-C₆ cycloalkyl, or C₅-C₆ cycloalkyl), R^{4B}-substituted or unsubstituted heterocycloalkyl (e.g., 3 to 8 membered heterocycloalkyl, 3 to 6 membered heterocycloalkyl, or 5 to 6 membered heterocycloalkyl), R^{4B}-substituted or unsubstituted aryl (e.g., C₆-C₁₀ aryl, C₁₀ aryl, or phenyl), or R^{4B}-substituted or unsubstituted heteroaryl (e.g., 5 to 10 membered heteroaryl, 5 to 9 membered heteroaryl, or 5 to 6 membered heteroaryl).

In embodiments, R^{2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2B} is R^{4B}-substituted or unsubstituted alkyl (e.g., C₁-C₈ alkyl, C₁-C₆ alkyl, or C₁-C₄ alkyl). In embodiments, R^{2B} is R^{4B}-substituted or unsubstituted heteroalkyl (e.g., 2 to 8 membered heteroalkyl, 2 to 6 membered heteroalkyl, or 2 to 4 membered heteroalkyl). In embodiments, R^{2B} is R^{4B}-substituted or unsubstituted cycloalkyl (e.g., C₃-C₈ cycloalkyl, C₃-C₆ cycloalkyl, or C₅-C₆ cycloalkyl 1). In embodiments, R^{2B} is R^{4B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2B} is R^{4B}-substituted or unsubstituted aryl (e.g., C₆-C₁₀ aryl, C₁₀ aryl, or phenyl). In embodiments, R^{2B} is R^{4B}-substituted or unsubstituted heteroaryl (e.g., 5 to 10 membered heteroaryl, 5 to 9 membered heteroaryl, or 5 to 6 membered heteroaryl).

R^{4B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5B}-substituted or unsubstituted alkyl (e.g., C₁-C₈ alkyl, C₁-C₆ alkyl, or C₁-C₄ alkyl), R^{5B}-substituted or unsubstituted heteroalkyl (e.g., 2 to 8 membered heteroalkyl, 2 to 6 membered heteroalkyl, or 2 to 4 membered heteroalkyl), R^{5B}-substituted or unsubstituted cycloalkyl (e.g., C₃-C₈ cycloalkyl, C₃-C₆ cycloalkyl, or C₅-C₆ cycloalkyl), R^{5B}-substituted or unsubstituted heterocycloalkyl (e.g., 3 to 8 membered heterocycloalkyl, 3 to 6 membered heterocycloalkyl, or 5 to 6 membered heterocycloalkyl), R^{5B}-substituted or unsubstituted aryl (e.g., C₆-C₁₀ aryl, C₁₀ aryl, or phenyl), or R^{5B}-substituted or unsubstituted heteroaryl (e.g., 5 to 10 membered heteroaryl, 5 to 9 membered heteroaryl, or 5 to 6 membered heteroaryl).

In embodiments, R^{4B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4B} is R^{5B}-substituted or unsubstituted alkyl (e.g., C₁-C₈ alkyl, C₁-C₆ alkyl, or C₁-C₄ alkyl). In embodiments, R^{4B} is R^{5B}-substituted or unsubstituted heteroalkyl (e.g., 2 to 8 membered heteroalkyl, 2 to 6 membered heteroalkyl, or 2 to 4 membered heteroalkyl). In embodiments, R^{4B} is R^{5B}-substituted or unsubstituted cycloalkyl (e.g., C₃-C₈ cycloalkyl, C₃-C₆ cycloalkyl, or C₅-C₆ cycloalkyl). In embodiments, R^{4B} is R^{5B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4B} is R^{5B}-substituted or unsubstituted aryl (e.g., C₆-C₁₀ aryl, C₁₀ aryl, or phenyl). In embodiments, R^{4B} is R^{5B}-substituted or unsubstituted heteroaryl (e.g., 5 to 10 membered heteroaryl, 5 to 9 membered heteroaryl, or 5 to 6 membered heteroaryl).

R^{5B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6B}-substituted or unsubstituted alkyl (e.g., C₁-C₈ alkyl, C₁-C₆ alkyl, or C₁-C₄ alkyl), R^{6B}-substituted or unsubstituted heteroalkyl (e.g., 2 to 8 membered heteroalkyl, 2 to 6 membered heteroalkyl, or 2 to 4 membered heteroalkyl), R^{6B}-substituted or unsubstituted cycloalkyl (e.g., C₃-C₈ cycloalkyl, C₃-C₆ cycloalkyl, or C₅-C₆ cycloalkyl), R^{6B}-substituted or unsubstituted heterocycloalkyl (e.g., 3 to 8 membered heterocycloalkyl, 3 to 6 membered heterocycloalkyl, or 5 to 6 membered heterocycloalkyl), R^{6B}-substituted or unsubstituted aryl (e.g., C₆-C₁₀ aryl, C₁₀ aryl, or phenyl), or R^{7B}-substituted or unsubstituted heteroaryl (e.g., 5 to 10 membered heteroaryl, 5 to 9 membered heteroaryl, or 5 to 6 membered heteroaryl).

In embodiments, R^{5B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5B} is R^{6B}-substituted or unsubstituted alkyl (e.g., C₁-C₈ alkyl, C₁-C₆ alkyl, or C₁-C₄ alkyl). In embodiments, R^{5B} is R^{6B}-substituted or unsubstituted heteroalkyl (e.g., 2 to 8 membered heteroalkyl, 2 to 6 membered heteroalkyl, or 2 to 4 membered heteroalkyl). In embodiments, R^{5B} is R^{6B}-substituted or unsubstituted cycloalkyl (e.g., C₃-C₈ cycloalkyl, C₃-C₆ cycloalkyl, or C₅-C₆ cycloalkyl). In embodiments, R^{5B} is R^{6B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5B} is R^{6B}-substituted or unsubstituted aryl (e.g., C₆-C₁₀ aryl, C₁₀ aryl, or phenyl). In embodiments, R^{5B} is R^{6B}-substituted or unsubstituted heteroaryl (e.g., 5 to 10 membered heteroaryl, 5 to 9 membered heteroaryl, or 5 to 6 membered heteroaryl).

R^{6B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6B} is independently unsubstituted alkyl (e.g., C₃-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2A} and R^{2B} substituents are joined together to form a substituted or unsubstituted heterocycloalkyl, substituted or unsubstituted aryl, or substituted or unsubstituted heteroaryl.

In embodiments, the compound has the formula (Ib):

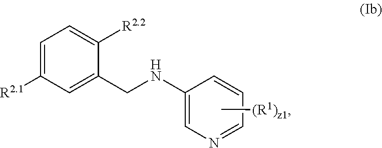

or a pharmaceutically acceptable salt thereof, wherein R¹ and z1 are as defined above, including embodiments thereof.

In embodiments, R^{2.1} is independently halogen, —CX^{2.1}₃, —CHX^{2.1}₂, —CH₂X^{2.1}, —OCX^{2.1}₃, —OCHX^{2.1}₂, —OCH₂X^{2.1}, —CN, —S(O)₂R^{2.1A}, —SR^{2.1A}, —S(O)R^{2.1A}, —SO₂NR^{2.1A}R^{2.1B}, —NHC(O)NR^{2.1A}R^{2.1B}, —N(O)₂, —NR^{2.1A}R^{2.1B}, —NHNR^{2.1A}R^{2.1B}, —C(O)R^{2.1A}, —C(O)—OR^{2.1A}, —C(O)NR^{2.1A}R^{2.1B}, —C(O)NHNR^{2.1A}R^{2.1B}, —OR^{2.1A}, —NR^{2.1A}SO₂R^{2.1B}, —NR^{2.1A}C(O)R^{2.1B}, —NR^{2.1A}C(O)OR^{2.1B}, —NR^{2.1A}OR^{2.1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{2.1A} and R^{2.1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX^{2.1}₃, —OCHX^{2.1}₂, —OCH₂X^{2.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2.1A} and R^{2.1B} substituents bonded to the same nitrogen atom may optionally be joined to form a substituted or unsubstituted heterocycloalkyl, substituted or unsubstituted aryl, or substituted or unsubstituted heteroaryl. X^{2.1} is independently halogen.

In embodiments, R^{2.2} is independently halogen, —CX^{2.2}₃, —CHX^{2.2}₂, —CH₂X^{2.2}, —OCX^{2.2}₃, —OCHX^{2.2}₂, —OCH₂X^{2.2}, —CN, —S(O)₂R^{2.2A}, —SR^{2.2A}, —S(O)R^{2.2A}, —SO₂NR^{2.2A}R^{2.2B}, —NHC(O)NR^{2.2A}R^{2.2B}, —N(O)₂, —NR^{2.2A}R^{2.2B}, —NHNR^{2.2A}R^{2.2B}, —C(O)R^{2.2A}, —C(O)—OR^{2.2A}, —C(O)NR^{2.2A}R^{2.2B}, —C(O)NHNR^{2.2A}R^{2.2B}, —OR^{2.2A}, —NR^{2.2A}SO₂R^{2.2B}, —NR^{2.2A}C(O)R^{2.2B}, —NR^{2.2A}C(O)OR^{2.2B}, —NR^{2.2A}OR^{2.2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{2.2A} and R^{2.2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX^{2.2}₃, —OCHX^{2.2}₂, —OCH₂X^{2.2}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2.2A} and R^{2.2B} substituents bonded to the same nitrogen atom may optionally be joined to form a substituted or unsubstituted heterocycloalkyl, substituted or unsubstituted aryl, or substituted or unsubstituted heteroaryl. X^{2.2} is independently halogen.

In embodiments, R^{2.1} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁴ 1-substituted or unsubstituted alkyl (e.g., C₁-C₈ alkyl, C₁-C₆ alkyl, or C₁-C₄ alkyl), R⁴ 1-substituted or unsubstituted heteroalkyl (e.g., 2 to 8 membered heteroalkyl, 2 to 6 membered heteroalkyl, or 2 to 4 membered heteroalkyl), R⁴-substituted or unsubstituted cycloalkyl (e.g., C₃-C₈ cycloalkyl, C₃-C₆ cycloalkyl, or C₅-C₆ cycloalkyl), R^{4.1}-substituted or unsubstituted heterocycloalkyl (e.g., 3 to 8 membered heterocycloalkyl, 3 to 6 membered heterocycloalkyl, or 5 to 6 membered heterocycloalkyl), R^{4.1}-substituted or unsubstituted aryl (e.g., C₆-C₁₀ aryl, C₁₀ aryl, or phenyl), or R⁴ 1-substituted or unsubstituted heteroaryl (e.g., 5 to 10 membered heteroaryl, 5 to 9 membered heteroaryl, or 5 to 6 membered heteroaryl).

In embodiments, R^{2.1} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.1} is R⁴ 1-substituted or unsubstituted alkyl (e.g., C₁-C₈ alkyl, C₁-C₆ alkyl, or C₁-C₄ alkyl). In embodiments, R^{2.1} is R^{4.1}-substituted or unsubstituted heteroalkyl (e.g., 2 to 8 membered heteroalkyl, 2 to 6 membered heteroalkyl, or 2 to 4 membered heteroalkyl). In embodiments, R^{2.1} is R^{4.1}-substituted or unsubstituted cycloalkyl (e.g., C₃-C₈ cycloalkyl, C₃-C₆ cycloalkyl, or C₅-C₆ cycloalkyl 1). In embodiments, R²¹ is R^{4.1}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²¹ is R^{4.1}-substituted or unsubstituted aryl (e.g., C₆-C₁₀ aryl, C₁₀ aryl, or phenyl). In embodiments, R^{2.1} is R^{4.1}-substituted or unsubstituted heteroaryl (e.g., 5 to 10 membered heteroaryl, 5 to 9 membered heteroaryl, or 5 to 6 membered heteroaryl).

R^{4.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.1}-substituted or unsubstituted alkyl (e.g., C₁-C₈ alkyl, C₁-C₆ alkyl, or C₁-C₄ alkyl), R^{5.1}-substituted or unsubstituted heteroalkyl (e.g., 2 to 8 membered heteroalkyl, 2 to 6 membered heteroalkyl, or 2 to 4 membered heteroalkyl), R^{5.1}-substituted or unsubstituted cycloalkyl (e.g., C₃-C₈ cycloalkyl, C₃-C₆ cycloalkyl, or C₅-C₆ cycloalkyl), R⁵ 1-substituted or unsubstituted heterocycloalkyl (e.g., 3 to 8 membered heterocycloalkyl, 3 to 6 membered heterocycloalkyl, or 5 to 6 membered heterocycloalkyl), R^{5.1}-substituted or unsubstituted aryl (e.g., C₆-C₁₀ aryl, C₁₀ aryl, or phenyl), or R^{5.1}-substituted or unsubstituted heteroaryl (e.g., 5 to 10 membered heteroaryl, 5 to 9 membered heteroaryl, or 5 to 6 membered heteroaryl).

In embodiments, R^{4.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.1} is R^{5.1}-substituted or unsubstituted alkyl (e.g., C₁-C₈ alkyl, C₁-C₆ alkyl, or C₁-C₄ alkyl). In embodiments, R^{4.1} is R^{5.1}-substituted or unsubstituted heteroalkyl (e.g., 2 to 8 membered heteroalkyl, 2 to 6 membered heteroalkyl, or 2 to 4 membered heteroalkyl). In embodiments, R^{4.1} is R^{5.1}-substituted or unsubstituted cycloalkyl (e.g., C₃-C₈ cycloalkyl, C₃-C₆ cycloalkyl, or C₅-C₆ cycloalkyl). In embodiments, R^{4.1} is R^{5.1}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4.1} is R^{5.1}-substituted or unsubstituted aryl (e.g., C₆-C₁₀ aryl, C₁₀ aryl, or phenyl). In embodiments, R^{4.1} is R^{5.1}-substituted or unsubstituted heteroaryl (e.g., 5 to 10 membered heteroaryl, 5 to 9 membered heteroaryl, or 5 to 6 membered heteroaryl).

R^{5.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.1}-substituted or unsubstituted alkyl (e.g., C₁-C₈ alkyl, C₁-C₆ alkyl, or C₁-C₄ alkyl), R^{6.1}-substituted or unsubstituted heteroalkyl (e.g., 2 to 8 membered heteroalkyl, 2 to 6 membered heteroalkyl, or 2 to 4 membered heteroalkyl), R^{6.1}-substituted or unsubstituted cycloalkyl (e.g., C₃-C₈ cycloalkyl, C₃-C₆ cycloalkyl, or C₅-C₆ cycloalkyl), R^{6.1}-substituted or unsubstituted heterocycloalkyl (e.g., 3 to 8 membered heterocycloalkyl, 3 to 6 membered heterocycloalkyl, or 5 to 6 membered heterocycloalkyl), R^{6.1}-substituted or unsubstituted aryl (e.g., C₆-C₁₀ aryl, C₁₀ aryl, or phenyl), or R^{6.1}-substituted or unsubstituted heteroaryl (e.g., 5 to 10 membered heteroaryl, 5 to 9 membered heteroaryl, or 5 to 6 membered heteroaryl).

In embodiments, R^{5.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.1} is R^{6.1}-substituted or unsubstituted alkyl (e.g., C₁-C₈ alkyl, C₁-C₆ alkyl, or C₁-C₄ alkyl). In embodiments, R^{5.1} is R^{6.1}-substituted or unsubstituted heteroalkyl (e.g., 2 to 8 membered heteroalkyl, 2 to 6 membered heteroalkyl, or 2 to 4 membered heteroalkyl). In embodiments, R^{5.1} is R^{6.1}-substituted or unsubstituted cycloalkyl (e.g., C₃-C₈ cycloalkyl, C₃-C₆ cycloalkyl, or C₅-C₆ cycloalkyl). In embodiments, R^{5.1} is R^{6.1}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.1} is R^{6.1}-substituted or unsubstituted aryl (e.g., C₆-C₁₀ aryl, C₁₀ aryl, or phenyl). In embodiments, R^{5.1} is R^{6.1}-substituted or unsubstituted heteroaryl (e.g., 5 to 10 membered heteroaryl, 5 to 9 membered heteroaryl, or 5 to 6 membered heteroaryl).

R^{6.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.1} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.1} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2.1} is OR^{2.1A} or R^{2.1A} and R^{2.1B} are joined together to form a substituted or unsubstituted heteroaryl. In embodiments, R^{2.1} is OR^{2.1A}. In embodiments, R^{2.1A} is substituted or unsubstituted alkyl. In embodiments, R^{2.1A} is substituted or unsubstituted C₁-C₃ alkyl. In embodiments, R^{2.1A} is substituted or unsubstituted C₁-C₄ alkyl. In embodiments, R^{2.1A} is unsubstituted C₁-C₂ alkyl. In embodiments, R^{2.1A} is substituted C₃-C₄ alkyl.

In embodiments, R^{2.1A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2.1A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2.1A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4.1A}-substituted or unsubstituted alkyl (e.g., C₁-C₈ alkyl, C₁-C₆ alkyl, or C₁-C₄ alkyl), R^{4.1A}-substituted or unsubstituted heteroalkyl (e.g., 2 to 8 membered heteroalkyl, 2 to 6 membered heteroalkyl, or 2 to 4 membered heteroalkyl), R^{4.1A}-substituted or unsubstituted cycloalkyl (e.g., C₃-C₈ cycloalkyl, C₃-C₆ cycloalkyl, or C₅-C₆ cycloalkyl), R^{4.1A}-substituted or unsubstituted heterocycloalkyl (e.g., 3 to 8 membered heterocycloalkyl, 3 to 6 membered heterocycloalkyl, or 5 to 6 membered heterocycloalkyl), R^{4.1A}-substituted or unsubstituted aryl (e.g., C₆-C₁₀ aryl, C₁₀ aryl, or phenyl), or R^{4.1A}-substituted or unsubstituted heteroaryl (e.g., 5 to 10 membered heteroaryl, 5 to 9 membered heteroaryl, or 5 to 6 membered heteroaryl).

In embodiments, R^{2.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.1A} is R^{4.1A}-substituted or unsubstituted alkyl (e.g., C₁-C₈ alkyl, C₁-C₆ alkyl, or C₁-C₄ alkyl). In embodiments, R^{2.A} is R^{4.1A}-substituted or unsubstituted heteroalkyl (e.g., 2 to 8 membered heteroalkyl, 2 to 6 membered heteroalkyl, or 2 to 4 membered heteroalkyl). In embodiments, R^{2.1A} is R^{4.1A}-substituted or unsubstituted cycloalkyl (e.g., C₃-C₈ cycloalkyl, C₃-C₆ cycloalkyl, or C₅-C₆ cycloalkyl 1). In embodiments, R^{2.1A} is R^{4.1A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2.1A} is R^{4.1A}-substituted or unsubstituted aryl (e.g., C₆-C₁₀ aryl, C₁₀ aryl, or phenyl). In embodiments, R^{2.1A} is R^{4.1A}-substituted or unsubstituted heteroaryl (e.g., 5 to 10 membered heteroaryl, 5 to 9 membered heteroaryl, or 5 to 6 membered heteroaryl).

R^{4.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.1A}-substituted or unsubstituted alkyl (e.g., C₁-C₈ alkyl, C₁-C₆ alkyl, or C₁-C₄ alkyl), R^{5.1A}-substituted or unsubstituted heteroalkyl (e.g., 2 to 8 membered heteroalkyl, 2 to 6 membered heteroalkyl, or 2 to 4 membered heteroalkyl), R^{5.1A}-substituted or unsubstituted cycloalkyl (e.g., C₃-C₈ cycloalkyl, C₃-C₆ cycloalkyl, or C₅-C₆ cycloalkyl), R^{5.1A}-substituted or unsubstituted heterocycloalkyl (e.g., 3 to 8 membered heterocycloalkyl, 3 to 6 membered heterocycloalkyl, or 5 to 6 membered heterocycloalkyl), R^{5.1A}-substituted or unsubstituted aryl (e.g., C₆-C₁₀ aryl, C₁₀ aryl, or phenyl), or R^{5.1A}-substituted or unsubstituted heteroaryl (e.g., 5 to 10 membered heteroaryl, 5 to 9 membered heteroaryl, or 5 to 6 membered heteroaryl).

In embodiments, R^{4.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.1A} is R^{5.1A}-substituted or unsubstituted alkyl (e.g., C₁-C₈ alkyl, C₁-C₆ alkyl, or C₁-C₄ alkyl). In embodiments, R^{4.1A} is R^{5.1A}-substituted or unsubstituted heteroalkyl (e.g., 2 to 8 membered heteroalkyl, 2 to 6 membered heteroalkyl, or 2 to 4 membered heteroalkyl). In embodiments, R^{4.1A} is R^{5.1A}-substituted or unsubstituted cycloalkyl (e.g., C₃-C₈ cycloalkyl, C₃-C₆ cycloalkyl, or C₅-C₆ cycloalkyl). In embodiments, R^{4.1A} is R^{5.1}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4.1A} is R^{5.1A}-substituted or unsubstituted aryl (e.g., C₆-C₁₀ aryl, C₁₀ aryl, or phenyl). In embodiments, R^{4.1A} is R^{5.1A}-substituted or unsubstituted heteroaryl (e.g., 5 to 10 membered heteroaryl, 5 to 9 membered heteroaryl, or 5 to 6 membered heteroaryl).

R^{5.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.1A}-substituted or unsubstituted alkyl (e.g., C₁-C₈ alkyl, C₁-C₆ alkyl, or C₁-C₄ alkyl), R^{6.1A}-substituted or unsubstituted heteroalkyl (e.g., 2 to 8 membered heteroalkyl, 2 to 6 membered heteroalkyl, or 2 to 4 membered heteroalkyl), R^{6.1A}-substituted or unsubstituted cycloalkyl (e.g., C₃-C₈ cycloalkyl, C₃-C₆ cycloalkyl, or C₅-C₆ cycloalkyl), R^{6.1A}-substituted or unsubstituted heterocycloalkyl (e.g., 3 to 8 membered heterocycloalkyl, 3 to 6 membered heterocycloalkyl, or 5 to 6 membered heterocycloalkyl), R^{6.1A}-substituted or unsubstituted aryl (e.g., C₆-C₁₀ aryl, C₁₀ aryl, or phenyl), or R^{6.1A}-substituted or unsubstituted heteroaryl (e.g., 5 to 10 membered heteroaryl, 5 to 9 membered heteroaryl, or 5 to 6 membered heteroaryl).

In embodiments, R^{5.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.1A} is R^{6.1A}-substituted or unsubstituted alkyl (e.g., C₁-C₈ alkyl, C₁-C₆ alkyl, or C₁-C₄ alkyl). In embodiments, R^{5.1A} is R^{6.1A}-substituted or unsubstituted heteroalkyl (e.g., 2 to 8 membered heteroalkyl, 2 to 6 membered heteroalkyl, or 2 to 4 membered heteroalkyl). In embodiments, R^{5.1A} is R^{6.1A}-substituted or unsubstituted cycloalkyl (e.g., C₃-C₈ cycloalkyl, C₃-C₆ cycloalkyl, or C₅-C₆ cycloalkyl). In embodiments, R^{5.1A} is R^{6.1A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.1A} is R^{6.1A}-substituted or unsubstituted aryl (e.g., C₆-C₁₀ aryl, C₁₀ aryl, or phenyl). In embodiments, R^{5.1A} is R^{6.1A}-substituted or unsubstituted heteroaryl (e.g., 5 to 10 membered heteroaryl, 5 to 9 membered heteroaryl, or 5 to 6 membered heteroaryl).

R^{6.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.1A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.1A} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2.1B} is hydrogen, —F, —Cl, Br, —I, —CF₃, —CHF₂, —CH₂F, —CCl₃, —CHCl₂, —CH₂C₁, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂C₁, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2.1B} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4.1B}-substituted or unsubstituted alkyl (e.g., C₁-C₈ alkyl, C₁-C₆ alkyl, or C₁-C₄ alkyl), R^{4.1B}-substituted or unsubstituted heteroalkyl (e.g., 2 to 8 membered heteroalkyl, 2 to 6 membered heteroalkyl, or 2 to 4 membered heteroalkyl), R^{4.1B}-substituted or unsubstituted cycloalkyl (e.g., C₃-C₈ cycloalkyl, C₃-C₆ cycloalkyl, or C₅-C₆ cycloalkyl), R^{4.1B}-substituted or unsubstituted heterocycloalkyl (e.g., 3 to 8 membered heterocycloalkyl, 3 to 6 membered heterocycloalkyl, or 5 to 6 membered heterocycloalkyl), R^{4.1B}-substituted or unsubstituted aryl (e.g., C₆-C₁₀ aryl, C₁₀ aryl, or phenyl), or R^{4.1B}-substituted or unsubstituted heteroaryl (e.g., 5 to 10 membered heteroaryl, 5 to 9 membered heteroaryl, or 5 to 6 membered heteroaryl).

In embodiments, R^{2.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.1B} is R^{4.1B}-substituted or unsubstituted alkyl (e.g., C₁-C₈ alkyl, C₁-C₆ alkyl, or C₁-C₄ alkyl). In embodiments, R^{2.1B} is R^{4.1B}-substituted or unsubstituted heteroalkyl (e.g., 2 to 8 membered heteroalkyl, 2 to 6 membered heteroalkyl, or 2 to 4 membered heteroalkyl). In embodiments, R^{2.1B} is R^{4.1B}-substituted or unsubstituted cycloalkyl (e.g., C₃-C₈ cycloalkyl, C₃-C₆ cycloalkyl, or C₅-C₆ cycloalkyl 1). In embodiments, R^{2.1B} is R^{4.1B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2.1B} is R^{4.1B}-substituted or unsubstituted aryl (e.g., C₆-C₁₀ aryl, C₁₀ aryl, or phenyl). In embodiments, R^{2.1B} is R^{4.1B}-substituted or unsubstituted heteroaryl (e.g., 5 to 10 membered heteroaryl, 5 to 9 membered heteroaryl, or 5 to 6 membered heteroaryl).

R^{4.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.1B}-substituted or unsubstituted alkyl (e.g., C₁-C₈ alkyl, C₁-C₆ alkyl, or C₁-C₄ alkyl), R^{5.1B}-substituted or unsubstituted heteroalkyl (e.g., 2 to 8 membered heteroalkyl, 2 to 6 membered heteroalkyl, or 2 to 4 membered heteroalkyl), R^{5.1B}-substituted or unsubstituted cycloalkyl (e.g., C₃-C₈ cycloalkyl, C₃-C₆ cycloalkyl, or C₅-C₆ cycloalkyl), R^{5.1B}-substituted or unsubstituted heterocycloalkyl (e.g., 3 to 8 membered heterocycloalkyl, 3 to 6 membered heterocycloalkyl, or 5 to 6 membered heterocycloalkyl), R^{5.1B}-substituted or unsubstituted aryl (e.g., C₆-C₁₀ aryl, C₁₀ aryl, or phenyl), or R^{5.1B}-substituted or unsubstituted heteroaryl (e.g., 5 to 10 membered heteroaryl, 5 to 9 membered heteroaryl, or 5 to 6 membered heteroaryl).

In embodiments, R^{4.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.1B} is R^{5.1B}-substituted or unsubstituted alkyl (e.g., C₁-C₈ alkyl, C₁-C₆ alkyl, or C₁-C₄ alkyl). In embodiments, R^{4.1B} is R^{5.1B}-substituted or unsubstituted heteroalkyl (e.g., 2 to 8 membered heteroalkyl, 2 to 6 membered heteroalkyl, or 2 to 4 membered heteroalkyl). In embodiments, R^{4.1B} is R^{5.1B}-substituted or unsubstituted cycloalkyl (e.g., C₃-C₈ cycloalkyl, C₃-C₆ cycloalkyl, or C₅-C₆ cycloalkyl). In embodiments, R^{4.1B} is R^{5.1B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4.1B} is R^{5.1B}-substituted or unsubstituted aryl (e.g., C₆-C₁₀ aryl, C₁₀ aryl, or phenyl). In embodiments, R^{4.1B} is R^{5.1B}-substituted or unsubstituted heteroaryl (e.g., 5 to 10 membered heteroaryl, 5 to 9 membered heteroaryl, or 5 to 6 membered heteroaryl).

R^{5.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.1B}-substituted or unsubstituted alkyl (e.g., C₁-C₈ alkyl, C₁-C₆ alkyl, or C₁-C₄ alkyl), R^{6.1B}-substituted or unsubstituted heteroalkyl (e.g., 2 to 8 membered heteroalkyl, 2 to 6 membered heteroalkyl, or 2 to 4 membered heteroalkyl), R^{6.1B} substituted or unsubstituted cycloalkyl (e.g., C₃-C₈ cycloalkyl, C₃-C₆ cycloalkyl, or C₅-C₆ cycloalkyl), R^{6.1B}-substituted or unsubstituted heterocycloalkyl (e.g., 3 to 8 membered heterocycloalkyl, 3 to 6 membered heterocycloalkyl, or 5 to 6 membered heterocycloalkyl), R^{6.1B}-substituted or unsubstituted aryl (e.g., C₆-C₁₀ aryl, C₁₀ aryl, or phenyl), or R^{6.1B}-substituted or unsubstituted heteroaryl (e.g., 5 to 10 membered heteroaryl, 5 to 9 membered heteroaryl, or 5 to 6 membered heteroaryl).

In embodiments, R^{5.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.1B} is R^{6.1B}-substituted or unsubstituted alkyl (e.g., C₁-C₈ alkyl, C₁-C₆ alkyl, or C₁-C₄ alkyl). In embodiments, R^{5.1B} is R^{6.1B}-substituted or unsubstituted heteroalkyl (e.g., 2 to 8 membered heteroalkyl, 2 to 6 membered heteroalkyl, or 2 to 4 membered heteroalkyl). In embodiments, R^{5.1B} is R^{6.1B}-substituted or unsubstituted cycloalkyl (e.g., C₃-C₈ cycloalkyl, C₃-C₆ cycloalkyl, or C₅-C₆ cycloalkyl). In embodiments, R^{5.1B} is R^{6.1B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.1B} is R^{6.1B}-substituted or unsubstituted aryl (e.g., C₆-C₁₀ aryl, C₁₀ aryl, or phenyl). In embodiments, R^{5.1B} is R^{6.1B}-substituted or unsubstituted heteroaryl (e.g., 5 to 10 membered heteroaryl, 5 to 9 membered heteroaryl, or 5 to 6 membered heteroaryl).

R^{6.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.1B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.1B} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2.2} is independently halogen, —CX^{2.2}₃, —CHX^{2.2}₂, —CH₂X^{2.2}, —OCX^{2.2}₃, —OCHX^{2.2}₂, —OCH₂X^{2.2}, —CN, —S(O)₂R^{2.2A}, —SR^{2.2A}, —S(O)R^{2.2A}, —SO₂NR^{2.2A}R^{2.2B}, —NHC(O)NR^{2.2A}R^{2.2B}, —N(O)₂, —NR^{2.2A}R^{2.2B}, —NNR^{2.2A}R^{2.2B}, —C(O)R^{2.2A}, —C(O)—OR^{2.2A}, —C(O)NR^{2.2A}R^{2.2B}, —C(O)NHNR^{2.2A}R^{2.2B}, —OR^{2.2A}, —NR^{2.2A}SO₂R^{2.2B}, —NR^{2.2A}C(O)R^{2.2B}, —NR^{2.2A}C(O)OR^{2.2B}, —NR^{2.2A}OR^{2.2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{2.2A} and R^{2.2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX^{2.2}₃, —OCHX^{2.2}₂, —OCH₂X^{2.2}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2.2A} and R^{2.2B} substituents bonded to the same nitrogen atom may optionally be joined to form a substituted or unsubstituted heterocycloalkyl, substituted or unsubstituted aryl, or substituted or unsubstituted heteroaryl. X^{2.2} is independently halogen.

In embodiments, R^{2.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4.2}-substituted or unsubstituted alkyl (e.g., C₁-C₈ alkyl, C₁-C₆ alkyl, or C₁-C₄ alkyl), R^{4.2}-substituted or unsubstituted heteroalkyl (e.g., 2 to 8 membered heteroalkyl, 2 to 6 membered heteroalkyl, or 2 to 4 membered heteroalkyl), R^{4.2}-substituted or unsubstituted cycloalkyl (e.g., C₃-C₈ cycloalkyl, C₃-C₆ cycloalkyl, or C₅-C₆ cycloalkyl), R^{4.2}-substituted or unsubstituted heterocycloalkyl (e.g., 3 to 8 membered heterocycloalkyl, 3 to 6 membered heterocycloalkyl, or 5 to 6 membered heterocycloalkyl), R^{4.2}-substituted or unsubstituted aryl (e.g., C₆-C₁₀ aryl, C₁₀ aryl, or phenyl), or R^{4.2}-substituted or unsubstituted heteroaryl (e.g., 5 to 10 membered heteroaryl, 5 to 9 membered heteroaryl, or 5 to 6 membered heteroaryl).

In embodiments, R^{2.2} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.2} is R^{4.2}-substituted or unsubstituted alkyl (e.g., C₁-C₈ alkyl, C₁-C₆ alkyl, or C₁-C₄ alkyl). In embodiments, R^{2.2} is R^{4.2}-substituted or unsubstituted heteroalkyl (e.g., 2 to 8 membered heteroalkyl, 2 to 6 membered heteroalkyl, or 2 to 4 membered heteroalkyl). In embodiments, R^{2.2} is R^{4.2}-substituted or unsubstituted cycloalkyl (e.g., C₃-C₈ cycloalkyl, C₃-C₆ cycloalkyl, or C₅-C₆ cycloalkyl 1). In embodiments, R²₂ is R^{4.2}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2.2} is R^{4.2}-substituted or unsubstituted aryl (e.g., C₆-C₁₀ aryl, C₁₀ aryl, or phenyl). In embodiments, R²₂ is R^{4.2}-substituted or unsubstituted heteroaryl (e.g., 5 to 10 membered heteroaryl, 5 to 9 membered heteroaryl, or 5 to 6 membered heteroaryl).

R⁴² is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.2}-substituted or unsubstituted alkyl (e.g., C₁-C₈ alkyl, C₁-C₆ alkyl, or C₁-C₄ alkyl), R^{5.2}-substituted or unsubstituted heteroalkyl (e.g., 2 to 8 membered heteroalkyl, 2 to 6 membered heteroalkyl, or 2 to 4 membered heteroalkyl), R⁵²-substituted or unsubstituted cycloalkyl (e.g., C₃-C₈ cycloalkyl, C₃-C₆ cycloalkyl, or C₅-C₆ cycloalkyl), R^{5.2}-substituted or unsubstituted heterocycloalkyl (e.g., 3 to 8 membered heterocycloalkyl, 3 to 6 membered heterocycloalkyl, or 5 to 6 membered heterocycloalkyl), R⁵²-substituted or unsubstituted aryl (e.g., C₆-C₁₀ aryl, C₁₀ aryl, or phenyl), or R⁵²-substituted or unsubstituted heteroaryl (e.g., 5 to 10 membered heteroaryl, 5 to 9 membered heteroaryl, or 5 to 6 membered heteroaryl).

In embodiments, R^{4.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.2} is R^{5.2}-substituted or unsubstituted alkyl (e.g., C₁-C₈ alkyl, C₁-C₆ alkyl, or C₁-C₄ alkyl). In embodiments, R^{4.2} is R^{5.2}-substituted or unsubstituted heteroalkyl (e.g., 2 to 8 membered heteroalkyl, 2 to 6 membered heteroalkyl, or 2 to 4 membered heteroalkyl). In embodiments, R^{4.2} is R^{5.2}-substituted or unsubstituted cycloalkyl (e.g., C₃-C₈ cycloalkyl, C₃-C₆ cycloalkyl, or C₅-C₆ cycloalkyl). In embodiments, R⁴² is R⁵²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴² is R^{5.2}-substituted or unsubstituted aryl (e.g., C₆-C₁₀ aryl, C₁₀ aryl, or phenyl). In embodiments, R^{4.2} is R^{5.2}-substituted or unsubstituted heteroaryl (e.g., 5 to 10 membered heteroaryl, 5 to 9 membered heteroaryl, or 5 to 6 membered heteroaryl).

R^{5.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.2}-substituted or unsubstituted alkyl (e.g., C₁-C₈ alkyl, C₁-C₆ alkyl, or C₁-C₄ alkyl), R^{6.2}-substituted or unsubstituted heteroalkyl (e.g., 2 to 8 membered heteroalkyl, 2 to 6 membered heteroalkyl, or 2 to 4 membered heteroalkyl), R⁶²-substituted or unsubstituted cycloalkyl (e.g., C₃-C₈ cycloalkyl, C₃-C₆ cycloalkyl, or C₅-C₆ cycloalkyl), R^{6.2}-substituted or unsubstituted heterocycloalkyl (e.g., 3 to 8 membered heterocycloalkyl, 3 to 6 membered heterocycloalkyl, or 5 to 6 membered heterocycloalkyl), R^{6.2}-substituted or unsubstituted aryl (e.g., C₆-C₁₀ aryl, C₁₀ aryl, or phenyl), or R^{6.2}-substituted or unsubstituted heteroaryl (e.g., 5 to 10 membered heteroaryl, 5 to 9 membered heteroaryl, or 5 to 6 membered heteroaryl).

In embodiments, R^{5.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.2} is R^{6.2}-substituted or unsubstituted alkyl (e.g., C₁-C₈ alkyl, C₁-C₆ alkyl, or C₁-C₄ alkyl). In embodiments, R⁵² is R^{6.2}-substituted or unsubstituted heteroalkyl (e.g., 2 to 8 membered heteroalkyl, 2 to 6 membered heteroalkyl, or 2 to 4 membered heteroalkyl). In embodiments, R^{5.2} is R^{6.2}-substituted or unsubstituted cycloalkyl (e.g., C₃-C₈ cycloalkyl, C₃-C₆ cycloalkyl, or C₅-C₆ cycloalkyl). In embodiments, R^{5.2} is R^{6.2}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.2} is R^{6.2}-substituted or unsubstituted aryl (e.g., C₆-C₁₀ aryl, C₁₀ aryl, or phenyl). In embodiments, R^{5.2} is R^{6.2}-substituted or unsubstituted heteroaryl (e.g., 5 to 10 membered heteroaryl, 5 to 9 membered heteroaryl, or 5 to 6 membered heteroaryl).

R^{6.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.2} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.2} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2.2} is halogen. In embodiments, R^{2.2} is —F, —Cl, —Br, —I. In embodiments, R^{2.2} is unsubstituted C₃-C₈ cycloalkyl. In embodiments, R^{2.2} is unsubstituted cyclopropyl. In embodiments, R² is unsubstituted cyclobutyl. In embodiments, R²₂ is unsubstituted heteroaryl. In embodiments, R²₂ is benzo[d][1,3]dioxole.

In embodiments, R^{2.2A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2.2A} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4.2A}-substituted or unsubstituted alkyl (e.g., C₁-C₈ alkyl, C₁-C₆ alkyl, or C₁-C₄ alkyl), R^{4.2A}-substituted or unsubstituted heteroalkyl (e.g., 2 to 8 membered heteroalkyl, 2 to 6 membered heteroalkyl, or 2 to 4 membered heteroalkyl), R^{4.2A}-substituted or unsubstituted cycloalkyl (e.g., C₃-C₈ cycloalkyl, C₃-C₆ cycloalkyl, or C₅-C₆ cycloalkyl), R^{4.2A}-substituted or unsubstituted heterocycloalkyl (e.g., 3 to 8 membered heterocycloalkyl, 3 to 6 membered heterocycloalkyl, or 5 to 6 membered heterocycloalkyl), R^{4.2A}-substituted or unsubstituted aryl (e.g., O₆—C₁₀ aryl, C₁₀ aryl, or phenyl), or R^{4.2A}-substituted or unsubstituted heteroaryl (e.g., 5 to 10 membered heteroaryl, 5 to 9 membered heteroaryl, or 5 to 6 membered heteroaryl).

In embodiments, R^{2.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.2A} is R^{4.2A}-substituted or unsubstituted alkyl (e.g., C₁-C₈ alkyl, C₁-C₆ alkyl, or C₁-C₄ alkyl). In embodiments, R^{2.2A} is R^{4.2A}-substituted or unsubstituted heteroalkyl (e.g., 2 to 8 membered heteroalkyl, 2 to 6 membered heteroalkyl, or 2 to 4 membered heteroalkyl). In embodiments, R^{2.2A} is R^{4.2A}-substituted or unsubstituted cycloalkyl (e.g., C₃-C₈ cycloalkyl, C₃-C₆ cycloalkyl, or C₅-C₆ cycloalkyl 1). In embodiments, R^{2.2A} is R^{4.2A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2.2A} is R^{4.2A}-substituted or unsubstituted aryl (e.g., C₆-C₁₀ aryl, C₁₀ aryl, or phenyl). In embodiments, R^{2.2A} is R^{4.2A}-substituted or unsubstituted heteroaryl (e.g., 5 to 10 membered heteroaryl, 5 to 9 membered heteroaryl, or 5 to 6 membered heteroaryl).

R^{4.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.2A}-substituted or unsubstituted alkyl (e.g., C₁-C₈ alkyl, C₁-C₆ alkyl, or C₁-C₄ alkyl), R^{5.2A}-substituted or unsubstituted heteroalkyl (e.g., 2 to 8 membered heteroalkyl, 2 to 6 membered heteroalkyl, or 2 to 4 membered heteroalkyl), R^{5.2A}-substituted or unsubstituted cycloalkyl (e.g., C₃-C₈ cycloalkyl, C₃-C₆ cycloalkyl, or C₅-C₆ cycloalkyl), R^{5.2A}-substituted or unsubstituted heterocycloalkyl (e.g., 3 to 8 membered heterocycloalkyl, 3 to 6 membered heterocycloalkyl, or 5 to 6 membered heterocycloalkyl), R^{5.2A}-substituted or unsubstituted aryl (e.g., C₆-C₁₀ aryl, C₁₀ aryl, or phenyl), or R^{5.2A}-substituted or unsubstituted heteroaryl (e.g., 5 to 10 membered heteroaryl, 5 to 9 membered heteroaryl, or 5 to 6 membered heteroaryl).

In embodiments, R^{4.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.2A} is R^{5.2A}-substituted or unsubstituted alkyl (e.g., C₁-C₈ alkyl, C₁-C₆ alkyl, or C₁-C₄ alkyl). In embodiments, R^{4.2A} is R^{5.2A}-substituted or unsubstituted heteroalkyl (e.g., 2 to 8 membered heteroalkyl, 2 to 6 membered heteroalkyl, or 2 to 4 membered heteroalkyl). In embodiments, R^{4.2A} is R^{5.2A}-substituted or unsubstituted cycloalkyl (e.g., C₃-C₈ cycloalkyl, C₃-C₆ cycloalkyl, or C₅-C₆ cycloalkyl). In embodiments, R^{4.2A} is R⁵²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4.2A} is R^{5.2A}-substituted or unsubstituted aryl (e.g., C₆-C₁₀ aryl, C₁₀ aryl, or phenyl). In embodiments, R^{4.2A} is R^{5.2A}-substituted or unsubstituted heteroaryl (e.g., 5 to 10 membered heteroaryl, 5 to 9 membered heteroaryl, or 5 to 6 membered heteroaryl).

R^{5.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.2A}-substituted or unsubstituted alkyl (e.g., C₁-C₈ alkyl, C₁-C₆ alkyl, or C₁-C₄ alkyl), R^{6.2A}-substituted or unsubstituted heteroalkyl (e.g., 2 to 8 membered heteroalkyl, 2 to 6 membered heteroalkyl, or 2 to 4 membered heteroalkyl), R^{6.2A}-substituted or unsubstituted cycloalkyl (e.g., C₃-C₈ cycloalkyl, C₃-C₆ cycloalkyl, or C₅-C₆ cycloalkyl), R^{6.2A}-substituted or unsubstituted heterocycloalkyl (e.g., 3 to 8 membered heterocycloalkyl, 3 to 6 membered heterocycloalkyl, or 5 to 6 membered heterocycloalkyl), R^{6.2A}-substituted or unsubstituted aryl (e.g., C₆-C₁₀ aryl, C₁₀ aryl, or phenyl), or R^{6.2A}-substituted or unsubstituted heteroaryl (e.g., 5 to 10 membered heteroaryl, 5 to 9 membered heteroaryl, or 5 to 6 membered heteroaryl).

In embodiments, R^{5.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.2A} is R^{6.2A}-substituted or unsubstituted alkyl (e.g., C₁-C₈ alkyl, C₁-C₆ alkyl, or C₁-C₄ alkyl). In embodiments, R^{5.2A} is R^{6.2A}-substituted or unsubstituted heteroalkyl (e.g., 2 to 8 membered heteroalkyl, 2 to 6 membered heteroalkyl, or 2 to 4 membered heteroalkyl). In embodiments, R^{5.2A} is R^{6.2A}-substituted or unsubstituted cycloalkyl (e.g., C₃-C₈ cycloalkyl, C₃-C₆ cycloalkyl, or C₅-C₆ cycloalkyl). In embodiments, R^{5.2A} is R^{6.2A}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.2A} is R^{6.2A}-substituted or unsubstituted aryl (e.g., C₆-C₁₀ aryl, C₁₀ aryl, or phenyl). In embodiments, R^{5.2A} is R^{6.2A}-substituted or unsubstituted heteroaryl (e.g., 5 to 10 membered heteroaryl, 5 to 9 membered heteroaryl, or 5 to 6 membered heteroaryl).

R^{6.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.2A} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.2A} is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2.2B} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R^{2.2B} is hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R^{2.2B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{4.2B}-substituted or unsubstituted alkyl (e.g., C₁-C₈ alkyl, C₁-C₆ alkyl, or C₁-C₄ alkyl), R^{4.2B}-substituted or unsubstituted heteroalkyl (e.g., 2 to 8 membered heteroalkyl, 2 to 6 membered heteroalkyl, or 2 to 4 membered heteroalkyl), R^{4.2B}-substituted or unsubstituted cycloalkyl (e.g., C₃-C₈ cycloalkyl, C₃-C₆ cycloalkyl, or C₅-C₆ cycloalkyl), R^{4.2B}-substituted or unsubstituted heterocycloalkyl (e.g., 3 to 8 membered heterocycloalkyl, 3 to 6 membered heterocycloalkyl, or 5 to 6 membered heterocycloalkyl), R^{4.2B}-substituted or unsubstituted aryl (e.g., C₆-C₁₀ aryl, C₁₀ aryl, or phenyl), or R^{4.2B}-substituted or unsubstituted heteroaryl (e.g., 5 to 10 membered heteroaryl, 5 to 9 membered heteroaryl, or 5 to 6 membered heteroaryl).

In embodiments, R^{2.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{2.2B} is R^{42B}-substituted or unsubstituted alkyl (e.g., C₁-C₈ alkyl, C₁-C₆ alkyl, or C₁-C₄ alkyl). In embodiments, R^{2.2B} is R^{4.2B}-substituted or unsubstituted heteroalkyl (e.g., 2 to 8 membered heteroalkyl, 2 to 6 membered heteroalkyl, or 2 to 4 membered heteroalkyl). In embodiments, R^{22B} is R^{4.2B}-substituted or unsubstituted cycloalkyl (e.g., C₃-C₈ cycloalkyl, C₃-C₆ cycloalkyl, or C₅-C₆ cycloalkyl 1). In embodiments, R^{2.2B} is R^{4.2B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{22B} is R^{4.2B}-substituted or unsubstituted aryl (e.g., C₆-C₁₀ aryl, C₁₀ aryl, or phenyl). In embodiments, R^{22B} is R^{4.2B}-substituted or unsubstituted heteroaryl (e.g., 5 to 10 membered heteroaryl, 5 to 9 membered heteroaryl, or 5 to 6 membered heteroaryl).

R^{4.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{5.2B}-substituted or unsubstituted alkyl (e.g., C₁-C₈ alkyl, C₁-C₆ alkyl, or C₁-C₄ alkyl), R^{5.2B}-substituted or unsubstituted heteroalkyl (e.g., 2 to 8 membered heteroalkyl, 2 to 6 membered heteroalkyl, or 2 to 4 membered heteroalkyl), R^{5.2B}-substituted or unsubstituted cycloalkyl (e.g., C₃-C₈ cycloalkyl, C₃-C₆ cycloalkyl, or C₅-C₆ cycloalkyl), R^{5.2B}-substituted or unsubstituted heterocycloalkyl (e.g., 3 to 8 membered heterocycloalkyl, 3 to 6 membered heterocycloalkyl, or 5 to 6 membered heterocycloalkyl), R^{5.2B}-substituted or unsubstituted aryl (e.g., C₆-C₁₀ aryl, C₁₀ aryl, or phenyl), or R^{5.2B}-substituted or unsubstituted heteroaryl (e.g., 5 to 10 membered heteroaryl, 5 to 9 membered heteroaryl, or 5 to 6 membered heteroaryl).

In embodiments, R^{4.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{4.2B} is R^{5.2B}-substituted or unsubstituted alkyl (e.g., C₁-C₈ alkyl, C₁-C₆ alkyl, or C₁-C₄ alkyl). In embodiments, R^{4.2B} is R^{5.2B}-substituted or unsubstituted heteroalkyl (e.g., 2 to 8 membered heteroalkyl, 2 to 6 membered heteroalkyl, or 2 to 4 membered heteroalkyl). In embodiments, R^{4.2B} is R^{5.2B}-substituted or unsubstituted cycloalkyl (e.g., C₃-C₈ cycloalkyl, C₃-C₆ cycloalkyl, or C₅-C₆ cycloalkyl). In embodiments, R^{4.2B} is R^{5.2B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{4.2B} is R^{5.2B}-substituted or unsubstituted aryl (e.g., C₆-C₁₀ aryl, C₁₀ aryl, or phenyl). In embodiments, R^{4.2B} is R^{5.2B}-substituted or unsubstituted heteroaryl (e.g., 5 to 10 membered heteroaryl, 5 to 9 membered heteroaryl, or 5 to 6 membered heteroaryl).

R^{5.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{6.2B}-substituted or unsubstituted alkyl (e.g., C₁-C₈ alkyl, C₁-C₆ alkyl, or C₁-C₄ alkyl), R^{6.2B}-substituted or unsubstituted heteroalkyl (e.g., 2 to 8 membered heteroalkyl, 2 to 6 membered heteroalkyl, or 2 to 4 membered heteroalkyl), R^{6.2B}-substituted or unsubstituted cycloalkyl (e.g., C₃-C₈ cycloalkyl, C₃-C₆ cycloalkyl, or C₅-C₆ cycloalkyl), R^{6.2B}-substituted or unsubstituted heterocycloalkyl (e.g., 3 to 8 membered heterocycloalkyl, 3 to 6 membered heterocycloalkyl, or 5 to 6 membered heterocycloalkyl), R^{6.2B}-substituted or unsubstituted aryl (e.g., C₆-C₁₀ aryl, C₁₀ aryl, or phenyl), or R^{6.2B}-substituted or unsubstituted heteroaryl (e.g., 5 to 10 membered heteroaryl, 5 to 9 membered heteroaryl, or 5 to 6 membered heteroaryl).

In embodiments, R^{5.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{5.2B} is R^{6.2B}-substituted or unsubstituted alkyl (e.g., C₁-C₈ alkyl, C₁-C₆ alkyl, or C₁-C₄ alkyl). In embodiments, R^{5.2B} is R^{6.2B}-substituted or unsubstituted heteroalkyl (e.g., 2 to 8 membered heteroalkyl, 2 to 6 membered heteroalkyl, or 2 to 4 membered heteroalkyl). In embodiments, R^{5.2B} is R^{6.2B}-substituted or unsubstituted cycloalkyl (e.g., C₃-C₈ cycloalkyl, C₃-C₆ cycloalkyl, or C₅-C₆ cycloalkyl). In embodiments, R^{5.2B} is R^{6.2B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5.2B} is R^{6.2B}-substituted or unsubstituted aryl (e.g., C₆-C₁₀ aryl, C₁₀ aryl, or phenyl). In embodiments, R^{5.2B} is R^{6.2B}-substituted or unsubstituted heteroaryl (e.g., 5 to 10 membered heteroaryl, 5 to 9 membered heteroaryl, or 5 to 6 membered heteroaryl).

R^{6.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{6.2B} is independently halogen, —CF₃, —CCl₃, —CBr₃, —Cl₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R^{6.2B} is independently unsubstituted alkyl (e.g., C₃-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, the compound is:

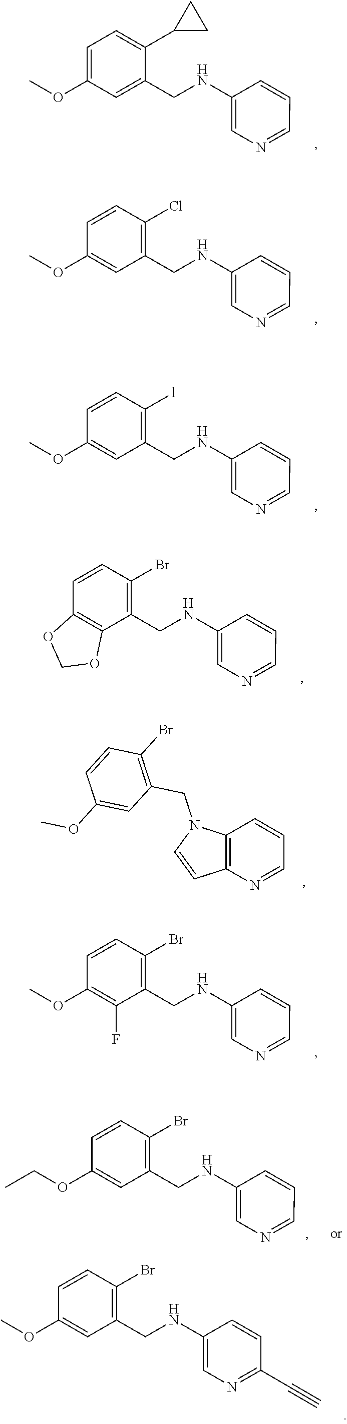

In embodiments, the compound is:

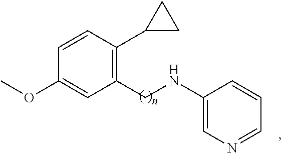

wherein n=1.

In embodiments, the compound is:

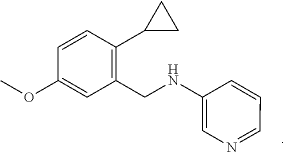 [SMILES: COc1ccc(C2CC2)c(CNc2cccnc2)c1]

In embodiments, the compound is:

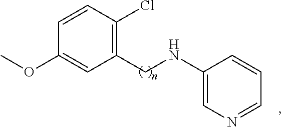

wherein n=1.

In embodiments, the compound is:

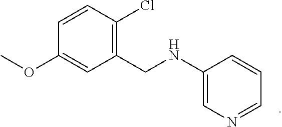 [SMILES: COc1ccc(Cl)c(CNc2cccnc2)c1]

In embodiments, the compound is:

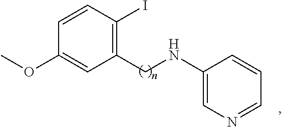

wherein n=1.

In embodiments, the compound is:

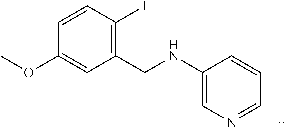 [SMILES: COc1ccc(I)c(CNc2cccnc2)c1]

In embodiments, the compound is:

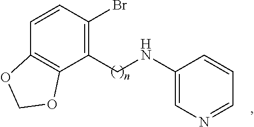

wherein n=1.

In embodiments, the compound is:

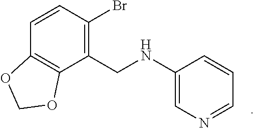 [SMILES: Brc1ccc2c(c1CNc1cccnc1)OCO2]

In embodiments, the compound is:

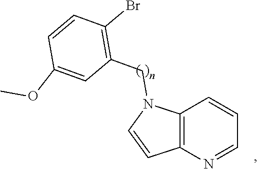

wherein n=1.

In embodiments, the compound is:

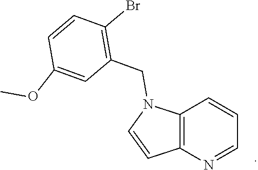 [SMILES: COc1ccc(Br)c(Cn2ccc3ncccc32)c1]

In embodiments, the compound is:

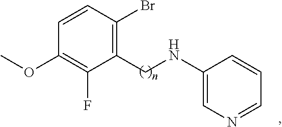

wherein n=1.

In embodiments, the compound is:

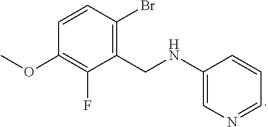 [SMILES: COc1ccc(Br)c(CNc2cccnc2)c1F]

In embodiments, the compound is:

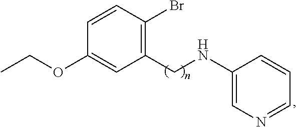

wherein n=1.

In embodiments, the compound is:

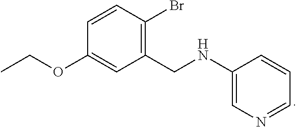 [SMILES: CCOc1ccc(Br)c(CNc2cccnc2)c1]

In embodiments, the compound is:

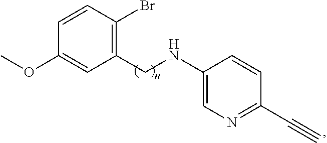

wherein n=1.

In embodiments, the compound is:

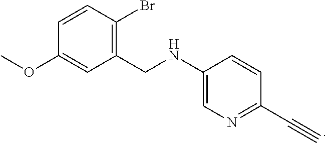 [SMILES: C#Cc1ccc(NCc2cc(OC)ccc2Br)cn1]

In an aspect, provided herein is a compound having the formula (II):

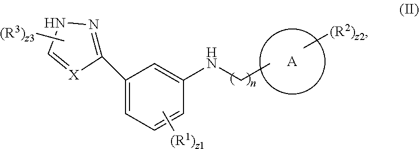

or a pharmaceutically acceptable salt thereof, wherein ring A, R¹, R², n, z1 and z2 are as defined above, including embodiments thereof.

X is independently —N or —CH.

R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{3A} and R^{3B} substituents bonded to the same nitrogen atom may optionally be joined to form a substituted or unsubstituted heterocycloalkyl, substituted or unsubstituted aryl, or substituted or unsubstituted heteroaryl. X³ is independently halogen. z3 is an integer from 0 to 2.

In embodiments, the compound has the formula (IIa):

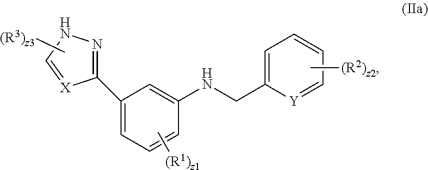

or a pharmaceutically acceptable salt thereof, wherein X, R¹, R², R³, z1, z2 and z3 are as defined above, including embodiments thereof.

Y is independently —N or —CH.

R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is halogen (e.g., —F, —Cl, Br, —I), —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B} NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, (e.g., —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X¹ is independently —F, —Cl, —Br, or —I.

In embodiments, R³ is —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R³ is —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R³ is substituted or unsubstituted C₁-C₃ alkyl. In embodiments, R³ is unsubstituted C₁-C₃ alkyl. In embodiments, R³ is methyl.

In embodiments, each R^{3A} and R^{3B} are independently hydrogen, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —COOH, —CONH₂, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₁₀, or phenyl), substituted (e.g., substituted with a substituent group, a size-limited substituent group, or lower substituent group) or unsubstituted heteroaryl (e.g., 5 to 10 membered, 5 to 9 membered, or 5 to 6 membered), or R^{3A} and R^{3B} substituents bonded to the same nitrogen atom may optionally be joined to form a substituted or unsubstituted heterocycloalkyl, substituted or unsubstituted aryl, or substituted or unsubstituted heteroaryl.

In embodiments, each R^{3A} and R^{3B} are independently hydrogen, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —COOH, —CONH₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, the compound has the formula (IIb):

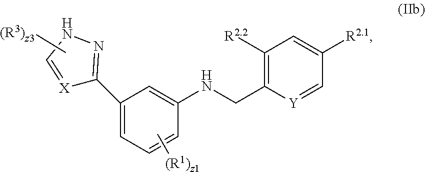

or a pharmaceutically acceptable salt thereof, wherein X, Y, R¹, R^{2.1}, R^{2.2}, R³, z1, and z3 are as defined above, including embodiments thereof.

In embodiments, the compound is:

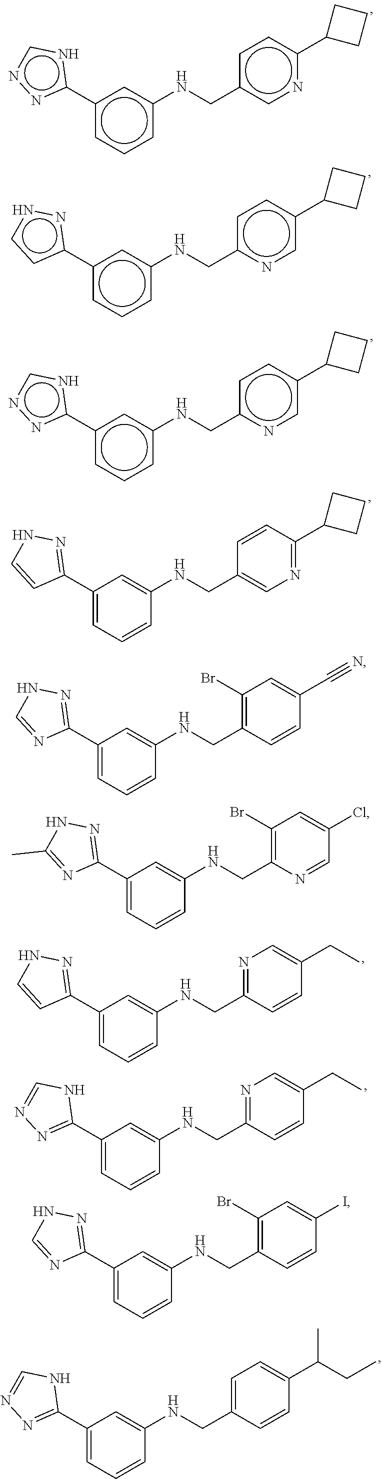

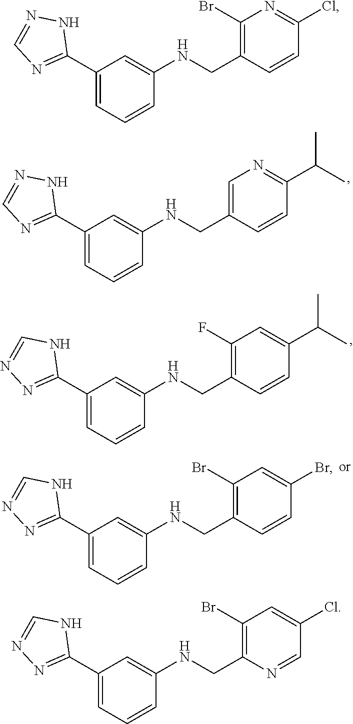 [SMILES: Clc1ccc(CNc2cccc(-c3ncn[nH]3)c2)c(Br)n1 ; CC(C)c1ccc(CNc2cccc(-c3ncn[nH]3)c2)cn1 ; CC(C)c1ccc(CNc2cccc(-c3nnc[nH]3)c2)c(F)c1 ; Brc1ccc(CNc2cccc(-c3nnc[nH]3)c2)c(Br)c1 ; Clc1cnc(CNc2cccc(-c3nnc[nH]3)c2)c(Br)c1]

In embodiments, the compound is:

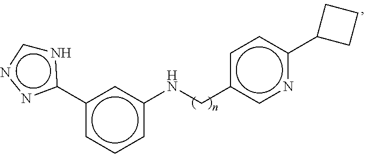

wherein n=1.

In embodiments, the compound is:

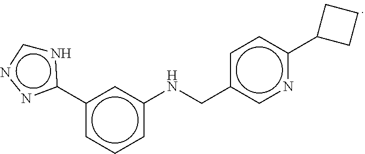

In embodiments, the compound is:

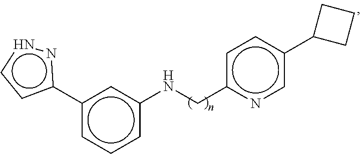

wherein n=1.

In embodiments, the compound is:

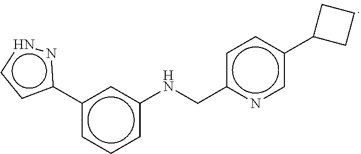

In embodiments, the compound is:

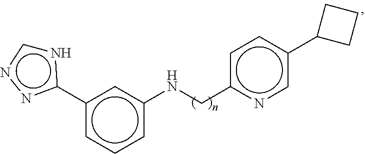

wherein n=1.

In embodiments, the compound is:

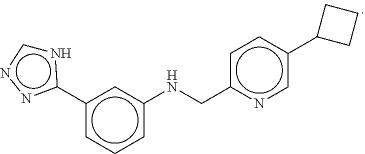

In embodiments, the compound is:

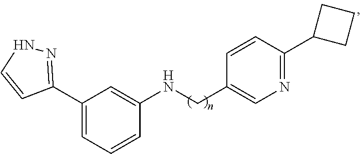

wherein n=1.

In embodiments, the compound is:

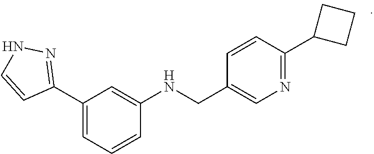 [SMILES: c1cc(NCc2ccc(C3CCC3)nc2)cc(-c2cc[nH]n2)c1]

In embodiments, the compound is:

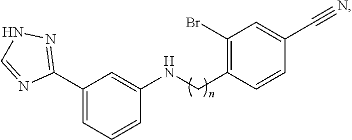

wherein n=1.

In embodiments, the compound is:

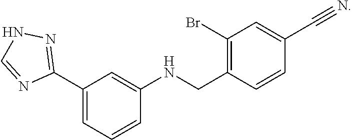 [SMILES: N#Cc1ccc(CNc2cccc(-c3nc[nH]n3)c2)c(Br)c1]

In embodiments, the compound is:

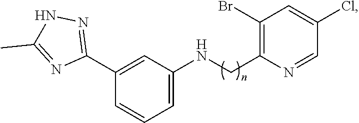

wherein n=1.

In embodiments, the compound is:

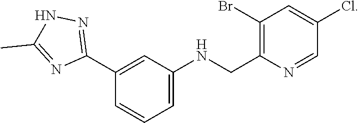 [SMILES: Cc1nc(-c2cccc(NCc3ncc(Cl)cc3Br)c2)n[nH]1]

In embodiments, the compound is:

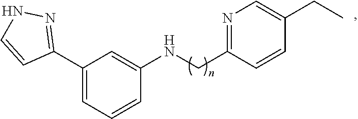

wherein n=1.

In embodiments, the compound is:

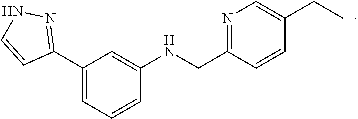 [SMILES: CCc1ccc(CNc2cccc(-c3cc[nH]n3)c2)nc1]

In embodiments, the compound is:

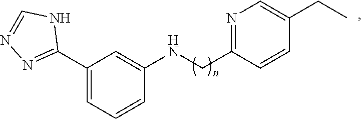

wherein n=1.

In embodiments, the compound is:

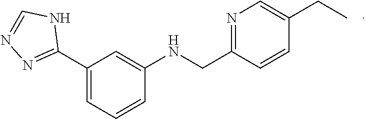 [SMILES: CCc1ccc(CNc2cccc(-c3nnc[nH]3)c2)nc1]

In embodiments, the compound is:

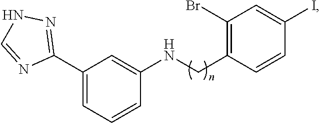

wherein n=1.

In embodiments, the compound is:

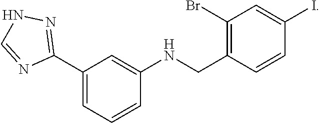 [SMILES: Brc1cc(I)ccc1CNc1cccc(-c2nc[nH]n2)c1]

In embodiments, the compound is:

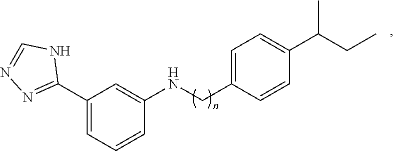

wherein n=1.

In embodiments, the compound is:

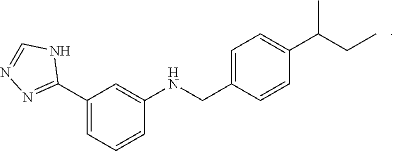 [SMILES: CCC(C)c1ccc(CNc2cccc(-c3nnc[nH]3)c2)cc1]

In embodiments, the compound is:

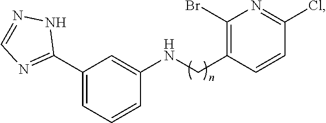

wherein n=1.

In embodiments, the compound is:

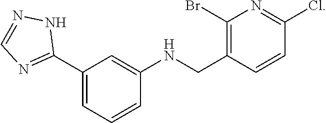 [SMILES: Clc1ccc(CNc2cccc(-c3ncn[nH]3)c2)c(Br)n1]

In embodiments, the compound is:

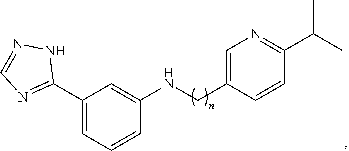

wherein n=1.

In embodiments, the compound is:

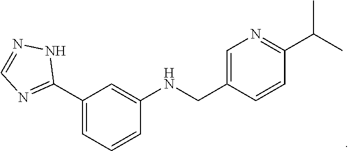 [SMILES: CC(C)c1ccc(CNc2cccc(-c3ncn[nH]3)c2)cn1]

In embodiments, the compound is:

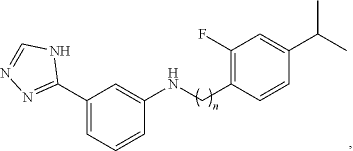

wherein n=1.

In embodiments, the compound is:

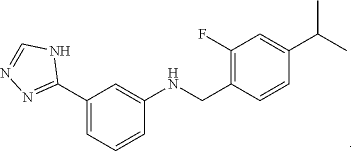 [SMILES: CC(C)c1ccc(CNc2cccc(-c3nnc[nH]3)c2)c(F)c1]

In embodiments, the compound is:

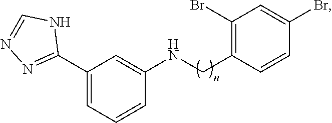

wherein n=1.

In embodiments, the compound is:

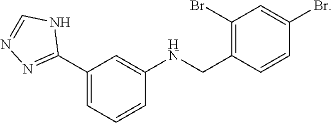 [SMILES: Brc1ccc(CNc2cccc(-c3nnc[nH]3)c2)c(Br)c1]

In embodiments, the compound is:

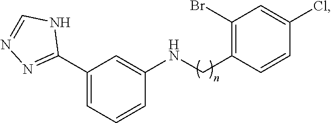

wherein n=1.

In embodiments, the compound is:

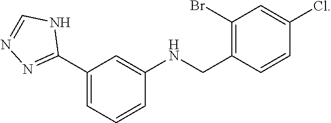 [SMILES: Clc1ccc(CNc2cccc(-c3nnc[nH]3)c2)c(Br)c1]

n is an integer from 0 to 5. In embodiments, n is 0. In embodiments, n is 1. In embodiments, n is 2. In embodiments, n is 3. In embodiments, n is 4. In embodiments, n is 5.

z1 is an integer from 0 to 2. In embodiments, z1 is 0. In embodiments, z1 is 1. In embodiments, z1 is 2.

z2 is an integer from 0 to 5. In embodiments, z2 is 0. In embodiments, z2 is 1. In embodiments, z2 is 2. In embodiments, z2 is 3. In embodiments, z2 is 4. In embodiments, z2 is 5.

z3 is an integer from 0 to 2. In embodiments, z3 is 0. In embodiments, z3 is 1. In embodiments, z3 is 2.

X¹ is halogen. In embodiments, X¹ is —F, —Cl, —Br, —I. In embodiments, X¹ is —F. In embodiments, X¹ is —Cl. In embodiments, X¹ is —Br. In embodiments, X¹ is —I.

X² is halogen. In embodiments, X² is —F, —Cl, —Br, —I. In embodiments, X² is —F. In embodiments, X² is —Cl. In embodiments, X² is —Br. In embodiments, X² is —I.

X^{2.1} is halogen. In embodiments, X^{2.1} is —F, —Cl, —Br, —I. In embodiments, X^{2.1} is —F. In embodiments, X^{2.1} is —Cl. In embodiments, X^{2.1} is —Br. In embodiments, X^{2.1} is —I.

X^{2.2} is halogen. In embodiments, X^{2.2} is —F, —Cl, —Br, —I. In embodiments, X^{2.2} is —F. In embodiments, X^{2.2} is —Cl. In embodiments, X^{2.2} is —Br. In embodiments, X^{2.2} is —I.

X³ is halogen. In embodiments, halogen is —F, —Cl, —Br, —I. In embodiments, X³ is —F. In embodiments, X³ is —Cl. In embodiments, X³ is —Br. In embodiments, X³ is —I.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R^{4.1}, R^{4.1A}, R^{4.1B}, R^{4.2}, R^{4.2A}, R^{4.2B}, R⁵, R^{5.1A}, R^{5B}, R^{5.1B}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R^{4.1}, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.1A}, R^{5.2B}, R⁶, R^{6A}, R^{6B}, R^{6.1}, R^{6.1A}, R^{6.1B}, R^{6.2}, R^{6.2A}, R^{6.2B} are independently unsubstituted alkyl, unsubstituted heteroalkyl, unsubstituted cycloalkyl, unsubstituted heterocycloalkyl, unsubstituted aryl, or unsubstituted heteroaryl.

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.1A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) or unsubstituted alkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) alkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R^{4.1}, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently unsubstituted alkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.A}, R^{6.1B}, R^{6.2}, R^{6.2A}, R^{6.2B} are independently substituted or unsubstituted alkyl (e.g., C₁-C₈ alkyl, C₁-C₆ alkyl, or C₁-C₄ alkyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted alkyl alkyl (e.g., C₁-C₈ alkyl, C₁-C₆ alkyl, or C₁-C₄ alkyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R^{4.1}, R^{4.1A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently unsubstituted alkyl alkyl (e.g., C₁-C₈ alkyl, C₁-C₆ alkyl, or C₁-C₄ alkyl).

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.1A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) or unsubstituted heteroalkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R^{4.1}, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.A}, R^{6.1B}, R^{6.2}, R^{6.2A}, R^{6.2B} are independently substituted (e.g., substituted with a substituent group, a size-limited substituent group, or lower substituent group) heteroalkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R⁴¹, R^{4.A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.A}, R^{6.1B}, R^{6.2}, R^{6.2A}, R^{6.2B} are independently unsubstituted heteroalkyl. In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted or unsubstituted heteroalkyl (e.g., 2 to 8 membered, 2 to 6 membered, 4 to 6 membered, 2 to 3 membered, or 4 to 5 membered). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2}B are independently substituted heteroalkyl (e.g., 2 to 8 membered, 2 to 6 membered, or 2 to 4 membered). In embodiments, R¹, R^{1A}, R^{1B}, R², R^{2A}, R^{2B}, R^{2.1}, R^{2.A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted heteroalkyl (e.g., 2 to 8 membered, 2 to 6 membered, or 2 to 4 membered).

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.A}, R^{4.1B}, R^{4.2}, R^{4.2A}, R^{4.2B}, R⁵, R^{5A}, R^{5B}, R⁵¹, R^{5.1A}, R^{5.1B}, R⁵², R^{5.1A}, R^{5.2B}, R⁶, R^{6A}, R^{6B}, R^{6.1}, R^{6.A}, R^{6.1B}, R^{6.2}, R^{6.2A}, R^{6.2B} are independently substituted (e.g., substituted with a substituent group, a size-limited substituent group, or lower substituent group) or unsubstituted cycloalkyl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) cycloalkyl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted cycloalkyl. In embodiments, R¹, R^{1A}, R^{1B}, R², R^{2A}, R^{2B}, R^{2.1}, R^{2.A}, R^{2.1B}, R^{2.1A}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted or unsubstituted cycloalkyl (e.g., C₃-C₈ cycloalkyl, C₃-C₆ cycloalkyl, or C₅-C₆ cycloalkyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted cycloalkyl (e.g., C₃-C₈ cycloalkyl, C₃-C₆ cycloalkyl, or C₅-C₆ cycloalkyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2}B are independently unsubstituted cycloalkyl (e.g., C₃-C₈ cycloalkyl, C₃-C₆ cycloalkyl, or C₅-C₆ cycloalkyl).

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵ 1 R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) or unsubstituted heterocycloalkyl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) heterocycloalkyl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2}B are independently an unsubstituted heterocycloalkyl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2}B are independently substituted or unsubstituted heterocycloalkyl (e.g., 3 to 8 membered, 3 to 6 membered, or 5 to 6 membered). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴, R^{4.1A}, R^{4.1B}, R^{4.2}, R^{4.2A}, R^{4.2B}, R⁵, R^{5A}, R^{5B}, R⁵, R^{5.1A}, R^{5.B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2}B are independently substituted heterocycloalkyl (e.g., 3 to 8 membered, 3 to 6 membered, or 5 to 6 membered). In embodiments, R¹, R^{1A}, R^{1B}, R², R^{2A}, R^{2B}, R^{2.1}, R^{2.1A}, R^{2.1B}, R^{2.2}, R^{2.A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted heterocycloalkyl (e.g., 3 to 8 membered, 3 to 6 membered, or 5 to 6 membered).

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵¹, R^{5.1A}, R^{5.1B}, R^{5.2}, R^{5.1A}, R^{5.2B}, R⁶, R^{6A}, R^{6B}, R⁶, R^{6.1A}, R^{6.1B}, R⁶. R^{6A}, R^{6.2B} are independently substituted (e.g., substituted with a substituent group, a size-limited substituent group, or lower substituent group) or unsubstituted aryl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.A}, R^{4.2B}, R⁵, R^{5A}, R^{5B}, R^{5.1}, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted (e.g., substituted with a substituent group, a size-limited substituent group, or lower substituent group) aryl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted aryl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted or unsubstituted aryl (e.g., C₆-C₁₀ aryl, C₁₀ aryl, or phenyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R⁶², R^{6.A}, R^{6.B} a independently substituted aryl (e.g., C₆-C₁₀ aryl, C₁₀ aryl, or phenyl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted aryl (e.g., C₆-C₁₀ aryl, C₁₀ aryl, or phenyl).

In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵¹, R^{5.1A}, R^{5.1B}, R^{5.2}, R^{5.1A}, R^{5.2B}, R⁶, R^{6A}, R^{6B}, R^{6.1}, R^{6.1A}, R^{6.1B}, R^{6.2}, R^{6.A}, R^{6.2B} are independently substituted (e.g., substituted with a substituent group, a size-limited substituent group, or lower substituent group) or unsubstituted heteroaryl. In embodiments, R¹, R^{1A}, R^{1B}, R², R^{2A}, R^{2B}R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{62A}, R^{6.2B} are independently substituted (e.g., substituted with a substituent group, a size-limited substituent group, or lower substituent group) heteroaryl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted heteroaryl. In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently substituted or unsubstituted heteroaryl (e.g., 5 to 10 membered heteroaryl, 5 to 9 membered heteroaryl, or 5 to 6 membered heteroaryl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R^{4.2}, R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{62B} are independently substituted heteroaryl (e.g., 5 to 10 membered heteroaryl, 5 to 9 membered heteroaryl, or 5 to 6 membered heteroaryl). In embodiments, R¹, R^{1A}, R^{1B}, R², R^{2A}, R^{2B}, R^{2.1}, R^{2.1A}, R^{2.1B}, R^{2.2}, R^{2.2A}, R^{2.2B}, R³, R^{3A}, R^{3B}, R⁴, R^{4A}, R^{4B}, R⁴¹, R^{4.1A}, R^{4.1B}, R⁴², R^{4.2A}, R^{4.2B}, R⁵, R^{5A}, R^{5B}, R⁵, R^{5.1A}, R^{5.1B}, R^{5.2}, R^{5.2A}, R^{5.2B}, R⁶, R^{6A}, R^{6B}, R^{6.1}, R^{6.1A}, R^{6.1B}, R^{6.2}, R^{6.2A}, R^{6.2B} are independently an unsubstituted heteroaryl (e.g., 5 to 10 membered heteroaryl, 5 to 9 membered heteroaryl, or 5 to 6 membered heteroaryl).

In embodiments, the compound has the formula as described elsewhere herein, for example within a table, claim or example.

### III. Pharmaceutical Compositions

In an aspect, there is provided a pharmaceutical composition, including a compound as described herein, including embodiments (e.g., structural Formulae (I), (Ia), (Ib), (Ic), (II), ((IIa), and (IIb)), and a pharmaceutically acceptable excipient.

The compounds as described herein of the present disclosure may be in the form of compositions suitable for administration to a subject. In general, such compositions are “pharmaceutical compositions” comprising a compound (e.g., compounds described herein) and one or more pharmaceutically acceptable or physiologically acceptable excipients (e.g., acceptable diluents or carriers). In certain embodiments, the compounds are present in a therapeutically effective amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The pharmaceutical compositions containing the active ingredient (e.g., an MT₂ agonist or an MT₁ inverse agonist described herein) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients that are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound described herein contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a Wnt/catenin signaling pathway inhibitor, including implants (e.g., implantable pumps) and catheter systems, slow injection pumps and devices, all of which are well known to the skilled artisan.

Depot injections, which are generally administered subcutaneously or intramuscularly, may also be utilized to release a compound disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor© EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compounds described herein in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compounds described herein contemplated by the present disclosure may be in the form of any other suitable pharmaceutical composition (e.g., sprays for nasal or inhalation use) currently known or developed in the future.

### IV. Methods of Use

In another aspect, provided herein is a method of increasing an MT₂ receptor activity in a subject in need thereof, the method comprising administering to said subject an effective amount of a compound disclosed herein, including embodiments (e.g., structural Formulae (I), (Ia), (Ib), and (Ic)), or a pharmaceutically acceptable salt thereof. In yet another aspect, provided herein is a method of treating depression in a subject in need thereof, the method comprising administering to said subject an effective amount of a compound disclosed herein, including embodiments (e.g., structural Formulae (I), (Ia), (Ib), and (Ic)), or a pharmaceutically acceptable salt thereof. In another aspect, provided herein is a method of treating an MT₂ receptor-related condition in a subject in need thereof, the method comprising administering to said subject an effective amount of a compound disclosed herein, including embodiments (e.g., structural Formulae (I), (Ia), and (Ib)), or a pharmaceutically acceptable salt thereof.

In embodiments, provided herein is a method of increasing MT₂ receptor activity in a subject in need thereof, the method comprising administering to said subject an effective amount of a compound disclosed herein, including embodiments (e.g., structural Formulae (I), (Ia), (Ib), and (Ic)), or a pharmaceutically acceptable salt thereof. In embodiments, the MT₂ receptor activity is increased by 1% to 100%. In embodiments, the MT₂ receptor activity is increased by 10% to 90%. In embodiments, the MT₂ receptor activity is increased by 20% to 80%. In embodiments, the MT₂ receptor activity is increased by 30% to 70%. In embodiments, the MT₂ receptor activity is increased by 40% to 60%. In embodiments, the MT₂ receptor activity is increased by 1%. In embodiments, the MT₂ receptor activity is increased by 5%. In embodiments, the MT₂ receptor activity is increased by 10%. In embodiments, the MT₂ receptor activity is increased by 20%. In embodiments, the MT₂ receptor activity is increased by 30%. In embodiments, the MT₂ receptor activity is increased by 40%. In embodiments, the MT₂ receptor activity is increased by 50%. In embodiments, the MT₂ receptor activity is increased by 60%. In embodiments, the MT₂ receptor activity is increased by 70%. In embodiments, the MT₂ receptor activity is increased by 80%. In embodiments, the MT₂ receptor activity is increased by 90%. In embodiments, the MT₂ receptor activity is increased by 100%.

In embodiments, an increase of the MT₂ receptor activity is a percentage of melatonin's response on the MT₂ receptor. In embodiments, the melatonin's response on the MT₂ receptor is defined as 100%.

In embodiments, provided herein is a method of treating depression in a subject in need thereof, the method comprising administering to said subject an effective amount of a compound disclosed herein, including embodiments (e.g., structural Formulae (I), (Ia), (Ib), and (Ic)), or a pharmaceutically acceptable salt thereof. In embodiments, the depression is mild depression, moderate depression or severe depression. In embodiments, the depression is mild depression. In embodiments, the depression is moderate depression. In embodiments, the depression is severe depression. In embodiments, the depression is associated with a sleep disorder. In embodiments, the depression is associated with a lack of sleep.

In embodiments, provided herein is a method of treating an MT₂ receptor-related condition in a subject in need thereof, the method comprising administering to said subject an effective amount of a compound disclosed herein, including embodiments (e.g., structural Formulae (I), (Ia), (Ib), and (Ic)), or a pharmaceutically acceptable salt thereof. In embodiments, the MT₂ receptor-related condition is a sleep disorder or somnipathy.

In some embodiments, the present disclosure provides methods for treating MT₂ receptor-related conditions. In some embodiments, the present disclosure provides methods for treating MT₂ receptor-related conditions with a compound described herein.

In embodiments, the MT₂ related condition is depression or somnipathy.

In embodiments, a method of treating the MT₂ related condition comprises administering to a patient in need thereof a therapeutically effective amount of a compound as described herein, including embodiments (e.g., structural Formulae (I), (Ia), (Ib), and (Ic), or a pharmaceutically acceptable salt thereof).

In embodiments, a method of treating the MT₂ related condition comprises administering to a patient in need thereof a therapeutically effective amount of a pharmaceutical composition as described herein, including embodiments (e.g., structural Formulae (I), (Ia), (Ib), and (Ic), or a pharmaceutically acceptable salt thereof).

In another aspect, provided herein is a method of advancing circadian phase, the method comprising administering to a subject in need thereof an effective amount of an inverse agonist of MT₁ receptor, including embodiments (e.g., structural Formulae (II), (IIa), and (IIb)), or a pharmaceutically acceptable salt thereof. In another aspect, provided herein is a method of decreasing of MT₁ receptor activity in a subject in need thereof, the method comprising administering to said subject an effective amount of a compound disclosed herein, including embodiments (e.g., structural Formulae (II), (IIa), and (IIb)), or a pharmaceutically acceptable salt thereof. In yet another aspect, provided herein is a method of reducing a signaling activity relative to basal signaling activity of the MT₁ receptor in a subject in need thereof, the method comprising administering to said subject an effective amount of a compound disclosed herein, including embodiments (e.g., structural Formulae (II), (IIa), and (IIb)), or a pharmaceutically acceptable salt thereof. In yet another aspect, provided herein is a method of treating an MT₁ receptor-related condition in a subject in need thereof, the method comprising administering to said subject an effective amount of a compound disclosed herein, including embodiments (e.g., structural Formulae (II), (IIa), and (IIb)), or a pharmaceutically acceptable salt thereof.

In embodiments, provided herein is a method of advancing circadian phase, the method comprising administering to a subject in need thereof an effective amount of an inverse agonist of the MT₁ receptor, including embodiments (e.g., structural Formulae (II), (IIa), and (IIb)), or a pharmaceutically acceptable salt thereof. In embodiments, the circadian phase is advanced by at least 30 minutes. In embodiments, the circadian phase is advanced by at least one hour. In embodiments, the circadian phase is advanced by at least 90 minutes. In embodiments, the circadian phase is advanced by at least two hours.

In embodiments, provided herein is a method of advancing circadian phase, the method comprising administering to a subject in need thereof an effective amount of an inverse agonist of the MT₁ receptor, including embodiments (e.g., structural Formulae (II), (IIa), and (IIb)), or a pharmaceutically acceptable salt thereof at dusk.

In embodiments, the MT₁ receptor inverse inhibitors (e.g., structural Formulae (II), (IIa), and (IIb)), or a pharmaceutically acceptable salt thereof, are chrono molecules with dual or multiple efficacies of modulating the circadian rhythm during a 24 hour period.

In embodiments, provided herein is a method of decreasing of the MT₁ receptor activity in a subject in need thereof, the method comprising administering to said subject an effective amount of a compound disclosed herein, including embodiments (e.g., structural Formulae (II), (IIa), and (IIb)), or a pharmaceutically acceptable salt thereof. In embodiments, the MT₁ receptor activity is reduced by 1% to 100%. In embodiments, the MT₁ receptor activity is reduced by 10% to 90%. In embodiments, the MT₁ receptor activity is reduced by 20% to 80%. In embodiments, the MT1 receptor activity is reduced by 30% to 70%. In embodiments, the MT₁ receptor activity is reduced by 40% to 60%. In embodiments, the MT₁ receptor activity is reduced by 1%. In embodiments, the MT₁ receptor activity is reduced by 10%. In embodiments, the MT₁ receptor activity is reduced by 20%. In embodiments, the MT₁ receptor activity is reduced by 30%. In embodiments, the MT₁ receptor activity is reduced by 40%. In embodiments, the MT₁ receptor activity is reduced by 50%. In embodiments, the MT₁ receptor activity is reduced by 60%. In embodiments, the MT₁ receptor activity is reduced by 70%. In embodiments, the MT₁ receptor activity is reduced by 80%. In embodiments, the MT₁ receptor activity is reduced by 90%. In embodiments, the MT₁ receptor activity is reduced by 100%.

In embodiments, provided herein is a method of reducing a signaling activity relative to a basal signaling activity of the MT₁ receptor in a subject in need thereof, the method comprising administering to said subject an effective amount of a compound disclosed herein, including embodiments (e.g., structural Formulae (II), (IIa), and (IIb)), or a pharmaceutically acceptable salt thereof. In embodiments, a signaling activity of the MT₁ receptor is reduced by 1% to 100%. In embodiments, a signaling activity of the MT₁ receptor is reduced by 10% to 90%. In embodiments, a signaling activity of the MT₁ receptor is reduced by 20% to 80%. In embodiments, a signaling activity the MT₁ receptor activity is reduced by 30% to 70%. In embodiments, a signaling activity of the MT₁ receptor is reduced by 40% to 60%. In embodiments, a signaling activity of the MT₁ receptor is reduced by 1%. In embodiments, a signaling activity of the MT₁ receptor is reduced by 10%. In embodiments, a signaling activity of the MT₁ receptor is reduced by 20%. In embodiments, a signaling activity of the MT₁ receptor is reduced by 30%. In embodiments, a signaling activity of the MT₁ receptor is reduced by 40%. In embodiments, a signaling activity of the MT₁ receptor is reduced by 50%. In embodiments, a signaling activity of the MT₁ receptor is reduced by 60%. In embodiments, a signaling activity of the MT₁ receptor is reduced by 70%. In embodiments, a signaling activity of the MT₁ receptor is reduced by 80%. In embodiments, a signaling activity of the MT₁ receptor is reduced by 90%. In embodiments, a signaling activity of the MT₁ receptor is reduced by 100%.

In embodiments, provided herein is a method of reducing a signaling activity relative to a basal signaling activity of the MT₂ receptor in a subject in need thereof, the method comprising administering to said subject an effective amount of a compound disclosed herein, including embodiments (e.g., structural Formulae (II), (IIa), and (IIb)), or a pharmaceutically acceptable salt thereof. In embodiments, a signaling activity of the MT₂ receptor is reduced by 1% to 100%. In embodiments, a signaling activity of the MT₂ receptor is reduced by 10% to 90%. In embodiments, a signaling activity of the MT₂ receptor is reduced by 20% to 80%. In embodiments, the MT₂ receptor activity is reduced by 30% to 70%. In embodiments, a signaling activity of the MT₂ receptor is reduced by 40% to 60%. In embodiments, a signaling activity of the MT₂ receptor is reduced by 1%. In embodiments, a signaling activity of the MT₂ receptor is reduced by 10%. In embodiments, a signaling activity of the MT₂ receptor is reduced by 20%. In embodiments, a signaling activity of the MT₂ receptor is reduced by 30%. In embodiments, a signaling activity of the MT₂ receptor is reduced by 40%. In embodiments, a signaling activity of the MT₂ receptor is reduced by 50%. In embodiments, a signaling activity of the MT₂ receptor is reduced by 60%. In embodiments, a signaling activity of the MT₂ receptor is reduced by 70%. In embodiments, a signaling activity of the MT₂ receptor is reduced by 80%. In embodiments, a signaling activity of the MT₂ receptor is reduced by 90%. In embodiments, a signaling activity of the MT₂ receptor is reduced by 100%.

In embodiments, provided herein is a method of treating an MT₁ receptor-related condition in a subject in need thereof, the method comprising administering to said subject an effective amount of a compound disclosed herein, including embodiments (e.g., structural Formulae (II), (IIa), and (IIb)), or a pharmaceutically acceptable salt thereof. In embodiments, the MT₁ receptor-related condition is a circadian rhythm sleep-wake cycle disorder. In embodiments, the circadian rhythm sleep-wake cycle disorder is delayed sleep-wake phase disorder, advanced sleep-wake phase disorder, irregular sleep-wake rhythm, non-24-hour sleep-wake rhythm disorder, shift work disorder, jet lag disorder or circadian rhythm sleep-wake disorder not otherwise specified.

In some embodiments, the present disclosure provides methods for treating MT₁ receptor-related conditions. In some embodiments, the present disclosure provides methods for treating MT₁ receptor-related conditions with a compound described herein.

In embodiments drawn to methods of treating an MT₁ related condition, the administration of a therapeutically effective amount of a compound described herein results in decreasing of the symptoms of the MT₁ related condition in comparison with the symptoms observed by not administering a therapeutically effective amount of the compound. In further embodiments drawn to methods of treating an MT₁ related condition, the administration of a therapeutically effective amount of a compound described herein results in elimination of the symptoms of the MT₁ related condition.

In embodiments, the MT₁ related condition is a circadian rhythm sleep-wake cycle disorder selected from delayed sleep-wake phase disorder, advanced sleep-wake phase disorder, irregular sleep-wake rhythm, non-24-hour sleep-wake rhythm disorder, shift work disorder, jet lag disorder and circadian rhythm sleep-wake disorder not otherwise specified.

In embodiments, a method of treating the MT₁ related condition comprises administering to a patient in need thereof a therapeutically effective amount of a compound as described herein, including embodiments (e.g., structural Formulae (II), (IIa), or (IIb), or a pharmaceutically acceptable salt thereof).

In embodiments, a method of treating the MT₁ related condition comprises administering to a patient in need thereof a therapeutically effective amount of a pharmaceutical composition as described herein, including embodiments (e.g., structural Formulae (II), (IIa), or (IIb), or a pharmaceutically acceptable salt thereof).

The present disclosure contemplates the administration of the compounds described herein, and compositions (e.g., pharmaceutical salts, pharmaceutical composition) thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. In embodiments, the administration is oral administration. In embodiments, the administration is parenteral administration.

The compounds of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the compounds of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the compounds contemplated by the present disclosure may be administered at dosage levels of about 0.01 mg/kg to about 50 mg/kg, about 0.1 mg/kg to about 25 mg/kg, about 0.1 mg/kg to about 20 mg/kg, about 0.5 mg/kg to about 15 mg/kg, about 1 mg/kg to about 10 mg/kg, about 2 mg/kg to about 8 mg/kg, about 3 mg/kg to about 6 mg/kg, or about 4 mg/kg to about 5 mg/kg of subject body weight per day, one, two, three, four or more times a day, to obtain the desired therapeutic effect. In embodiments, the compounds contemplated by the present disclosure may be administered at dosage levels of about 0.01 mg/kg, about 0.1 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg of subject body weight per day, one, two, three, four or more times a day, to obtain the desired therapeutic effect.

For administration of an oral agent, the compositions can be provided in the form of tablets, capsules and the like containing from 0.01 to 1000 milligrams of the active ingredient, particularly 0.01, 0.05, 0.1, 0.25, 0.5, 0.75, 1.0, 1.25, 1.5, 1.75, 2.0, 2.5, 5.0, 7.5, 10.0, 15.0, 20.0, 25.0, 50.0, 75.0, 100.0, 125.0, 150.0, 175.0, 200.0, 250.0, 300.0, 400.0, 500.0, 600.0, 750.0, 800.0, 900.0, and 1000.0 milligrams of the active ingredient.

A pharmaceutically acceptable carrier(s), diluent(s) and/or excipient(s) may be present in an amount of from about 0.1 g to about 2.0 g.

In embodiments, the dosage of the desired compound is contained in a “unit dosage form”. The phrase “unit dosage form” refers to physically discrete units, each unit including a predetermined amount of a compound (e.g., a compound described herein), sufficient to produce the desired effect. It will be appreciated that the parameters of a unit dosage form will depend on the particular agent and the effect to be achieved.

### V. Kits

In another aspect, provided herein is a kit including a compound described herein or pharmaceutical compositions thereof. The kits are generally in the form of a physical structure housing various components, as described below, and may be utilized, for example, in practicing the methods described above.

A kit may include one or more of the compounds disclosed herein (e.g., provided in a sterile container), which may be in the form of a pharmaceutical composition suitable for administration to a subject. In embodiments, the compound has the structure of Formulae (I), (Ia), (Ib), (Ic), (II), (IIa), or (IIb), or a pharmaceutically acceptable salt thereof. The compounds described herein can be provided in a form that is ready for use (e.g., a tablet or capsule) or in a form requiring, for example, reconstitution or dilution (e.g., a powder) prior to administration. When the compound is in a form that needs to be reconstituted or diluted by a user, the kit may also include diluents (e.g., sterile water), buffers, pharmaceutically acceptable excipients, and the like, packaged with, or separately from, the compound. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### NUMBERED EMBODIMENTS

Embodiment 1. A method of increasing melatonin type 2 (MT₂) receptor activity in a subject in need thereof, the method comprising administering to said subject an effective amount of a compound of formula (I):

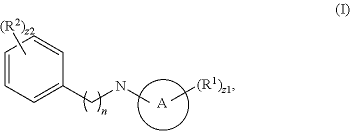

or a pharmaceutically acceptable salt thereof, wherein:
n is and integer from 0 to 5;
z1 is an integer from 0 to 2;
z2 is an integer from 0 to 5;

ring A is a substituted or unsubstituted aryl or heteroaryl;

R¹ is independently halogen, —CX¹₃, —CHX¹₂, —CH₂X¹, —OCX¹₃, —OCHX¹₂, —OCH₂X¹, —CN, —S(O)₂R^{A1}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{A1}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{2B}, —OR^{2A},

—NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl; and

X¹ and X² are independently halogen.

Embodiment 2. A method of treating depression in a subject in need thereof, the method comprising administering to said subject an effective amount of a compound of formula (I):

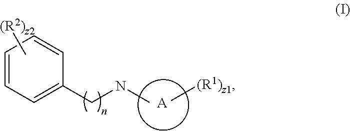

or a pharmaceutically acceptable salt thereof,
wherein:
n is and integer from 0 to 5;
z1 is an integer from 0 to 2;
z2 is an integer from 0 to 5;

ring A is a substituted or unsubstituted aryl or heteroaryl;

R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}

—NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl; and

X¹ and X² are independently halogen.

Embodiment 3. A method of treating an MT₂ receptor-related condition in a subject in need thereof, the method comprising administering to said subject an effective amount of a compound of formula (I):

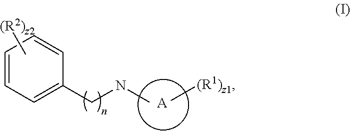

or a pharmaceutically acceptable salt thereof,
wherein:
n is and integer from 0 to 5;
z1 is an integer from 0 to 2;
z2 is an integer from 0 to 5;

ring A is a substituted or unsubstituted aryl or heteroaryl;

R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl; and

X¹ and X² are independently halogen.

Embodiment 4. The method of any one of embodiments 1 to 3, wherein the MT₂ receptor-related condition is somnipathy.

Embodiment 5. The method of any one of embodiments 1 to 4, wherein ring A is a

6-membered substituted or unsubstituted aryl or a 6-membered substituted or unsubstituted heteroaryl.

Embodiment 6. The method of any one of embodiments 1 to 5, wherein ring A is a substituted or unsubstituted phenyl or a substituted or unsubstituted pyridinyl.

Embodiment 7. The method of any one of embodiments 1 to 6, wherein ring A is a substituted or unsubstituted phenyl.

Embodiment 8. The method of any one of embodiments 1 to 6, wherein ring A is a substituted or unsubstituted pyridinyl.

Embodiment 9. The method of any one of embodiments 1 to 8, wherein the compound having formula (Ia) or (Ic):

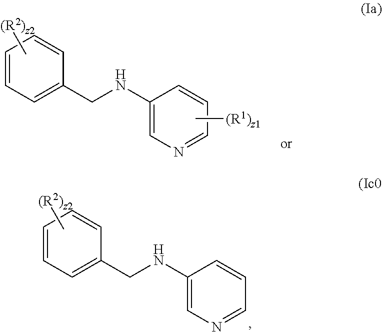

or a pharmaceutically acceptable salt thereof.

Embodiment 10. The method of any one of embodiments 1 to 9, wherein z1 is 0 or 1.

Embodiment 11. The method of any one of embodiments 1 to 10, wherein z2 is 2 or 3.

Embodiment 12. The method of any one of embodiments 1 to 11, wherein R¹ is —C≡C—.

Embodiment 13. The method of any one of embodiments 1 to 11, wherein R² is independently halogen, —OR^{2A}, or substituted or unsubstituted cycloalkyl.

Embodiment 14. The method of any one of embodiments 1 to 13, wherein R² is halogen.

Embodiment 15. The method of embodiment 14, wherein R² is —F, —Cl, or —Br.

Embodiment 16. The method of any one of embodiments 1 to 13, wherein R² is —OR^{2A}, wherein R^{2A} is a substituted or unsubstituted alkyl.

Embodiment 17. The method of embodiment 16, wherein R² is substituted or unsubstituted C₁-C₃ alkyl.

Embodiment 18. The method of any one of embodiments 1 to 13, wherein R² is a substituted or unsubstituted cycloalkyl.

Embodiment 19. The method of embodiment 18, wherein R² is unsubstituted C₃-C₅ cycloalkyl.

Embodiment 20. The method of any one of embodiments 1 to 19, wherein the compound is:

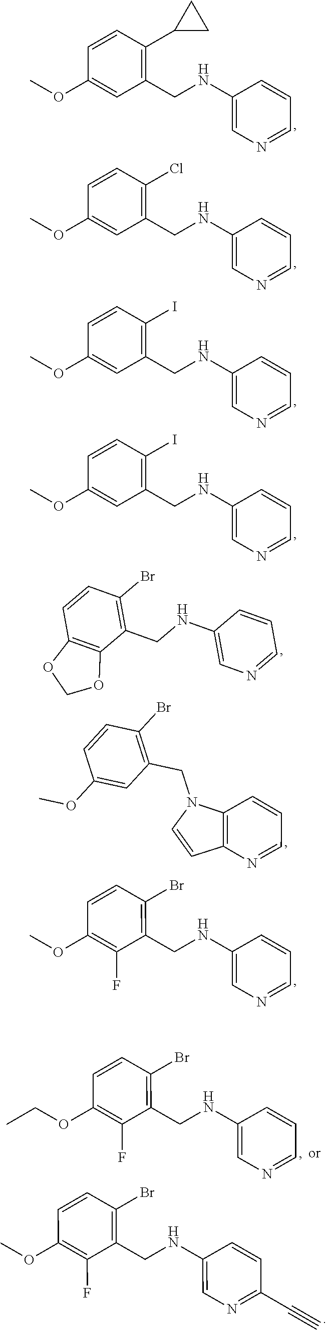

Embodiment 21. A method of advancing circadian phase comprising administering to a subject in need thereof an effective amount of an inverse agonist of melatonin type 1 (MT₁) receptor of formula (II):

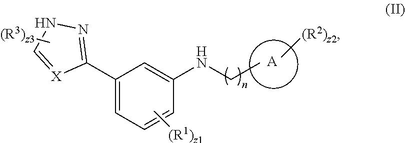

or a pharmaceutically acceptable salt thereof, wherein:
n is and integer from 0 to 5;
z1 is an integer from 0 to 2;
z2 is an integer from 0 to 5;
z3 is an integer from 0 to 2;
X is —N or —CH;

ring A is a substituted or unsubstituted aryl or heteroaryl;

R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NH—R^{2A}R^{2B}, —C(O)R^{2A}, —C(O)—OR^{2A}, —C(O)NR^{2A}R^{2B}, —C(O)NHNR^{2A}R^{B}, —OR^{2A}

—NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}

—NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl;

R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{3A} and R^{3B} substituents bonded to the same nitrogen atom may optionally be joined to form a substituted or unsubstituted heterocycloalkyl or substituted or unsubstituted heteroaryl; and

X¹, X² and X³ are independently halogen.

Embodiment 22. The method of embodiment 21, wherein the circadian phase is advanced by at least one hour.

Embodiment 23. A method of decreasing of MT₁ receptor activity in a subject in need thereof, the method comprising administering to said subject an effective amount of a compound of formula (II):

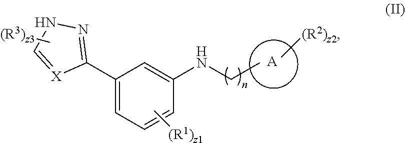

or a pharmaceutically acceptable salt thereof, wherein:
n is and integer from 0 to 5;
z1 is an integer from 0 to 2;
z2 is an integer from 0 to 5;
z3 is an integer from 0 to 2;
X is —N or —CH;

ring A is a substituted or unsubstituted aryl or heteroaryl;

R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂,

—NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl;

R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{3A} and R^{3B} substituents bonded to the same nitrogen atom may optionally be joined to form a substituted or unsubstituted heterocycloalkyl or substituted or unsubstituted heteroaryl; and

X¹, X² and X³ are independently halogen.

Embodiment 24. A method of treating an MT₁ receptor related condition in a subject in need thereof, the method comprising administering to said subject an effective amount of a compound of formula (II):

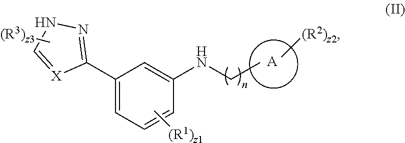

or a pharmaceutically acceptable salt thereof, wherein:
n is and integer from 0 to 5;
z1 is an integer from 0 to 2;
z2 is an integer from 0 to 5;
z3 is an integer from 0 to 2;
X is —N or —CH;

ring A is a substituted or unsubstituted aryl or heteroaryl;

R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A},

—NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B} NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl

R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom may optionally be joined to form a substituted or unsubstituted heterocycloalkyl or substituted or unsubstituted heteroaryl;

R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2A} and R^{2B} substituents bonded to the same nitrogen atom may optionally be joined to form a substituted or unsubstituted heterocycloalkyl or substituted or unsubstituted heteroaryl;

R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{3A} and R^{3B} substituents bonded to the same nitrogen atom may optionally be joined to form a substituted or unsubstituted heterocycloalkyl or substituted or unsubstituted heteroaryl; and

X¹, X² and X³ are independently halogen.

Embodiment 25. The method of any one of embodiments 21 to 24, wherein the MT₁ receptor related condition is a circadian rhythm sleep-wake cycle disorder.

Embodiment 26. The method of any one of embodiments 21 to 25, wherein the circadian rhythm sleep-wake cycle disorder is delayed sleep-wake phase disorder, advanced sleep-wake phase disorder, irregular sleep-wake rhythm, non-24-hour sleep-wake rhythm disorder, shift work disorder, jet lag disorder or circadian rhythm sleep-wake disorder not otherwise specified.

Embodiment 27. The method of any one of embodiments 21 to 26, wherein ring A is a 6-membered substituted or unsubstituted aryl or a 6-membered substituted or unsubstituted heteroaryl.

Embodiment 28. The method of any one of embodiments 21 to 27, wherein ring A is a substituted or unsubstituted phenyl or a substituted or unsubstituted pyridinyl.

Embodiment 29. The method of any one of embodiments 21 to 28, wherein ring A is a substituted or unsubstituted phenyl.

Embodiment 30. The method of any one of embodiments 21 to 28, wherein ring A is a substituted or unsubstituted pyridinyl.

Embodiment 31. The method of any one of embodiments 21 to 30 having formula (IIa):

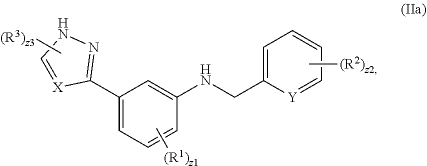

or a pharmaceutically acceptable salt thereof, wherein Y is —N or —CH.

Embodiment 32. The method of any one of embodiments 21 to 31, wherein z1 is 0.

Embodiment 33. The method of any one of embodiments 21 to 32, wherein z2 is 1 or 2.

Embodiment 34. The method of any one of embodiments 21 to 33, wherein z3 is 0 or 1.

Embodiment 35. The method of any one of embodiments 21 to 34, wherein R² is independently halogen, —CN, substituted or unsubstituted alkyl, or substituted or unsubstituted cycloalkyl.

Embodiment 36. The method of any one of embodiments 21 to 35, wherein R² is halogen.

Embodiment 37. The method of any one of embodiments 21 to 36, wherein R² is —F, —Cl, or —Br.

Embodiment 38. The method of any one of embodiments 21 to 35, wherein R² is —CN.

Embodiment 39. The method of any one of embodiments 21 to 35, wherein R² is a substituted or unsubstituted alkyl.

Embodiment 40. The method of any one of embodiments 21 to 35, wherein R² is C₁-C₄ substituted or unsubstituted alkyl.

Embodiment 41. The method of any one of embodiments 21 to 35, wherein R² is unsubstituted cycloalkyl.

Embodiment 42. The method of any one of embodiments 21 to 35, wherein R² is unsubstituted C₃-C₈ cycloalkyl.

Embodiment 43. The method of any one of embodiments 21 to 42, wherein R³ is a substituted or unsubstituted alkyl.

Embodiment 44. The method of any one of embodiments 21 to 43, wherein R³ is an unsubstituted C₁-C₃ alkyl.

Embodiment 45. The method of any one of embodiments 21 to 44, wherein X is —N.

Embodiment 46. The method of any one of embodiments 21 to 44, wherein X is —CH.

Embodiment 47. The method of any one of embodiments 31 to 46, wherein Y is —N.

Embodiment 48. The method of any one of embodiments 31 to 46, wherein Y is —CH.

Embodiment 49. The method of any one of embodiments 21 to 48, wherein the compound is:

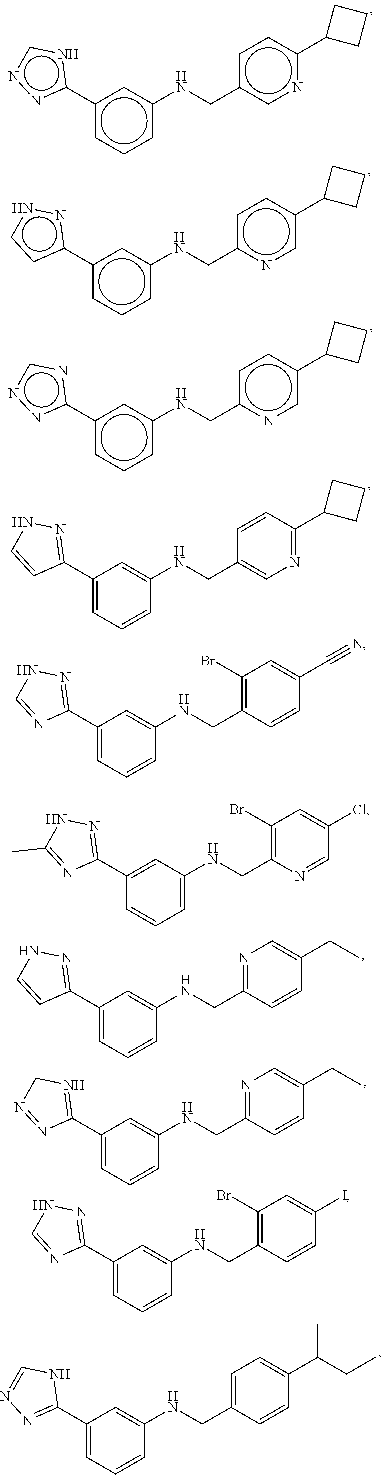

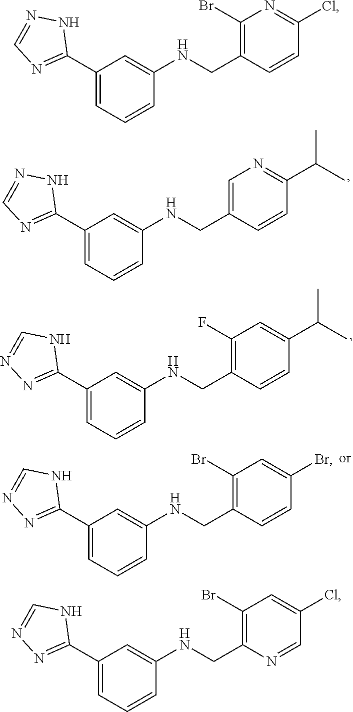 [SMILES: Clc1ccc(CNc2cccc(-c3ncn[nH]3)c2)c(Br)n1 ; CC(C)c1ccc(CNc2cccc(-c3ncn[nH]3)c2)cn1 ; CC(C)c1ccc(CNc2cccc(-c3nnc[nH]3)c2)c(F)c1 ; Brc1ccc(CNc2cccc(-c3nnc[nH]3)c2)c(Br)c1 ; Clc1cnc(CNc2cccc(-c3nnc[nH]3)c2)c(Br)c1]

Embodiment 50. A compound selected from the group consisting of:

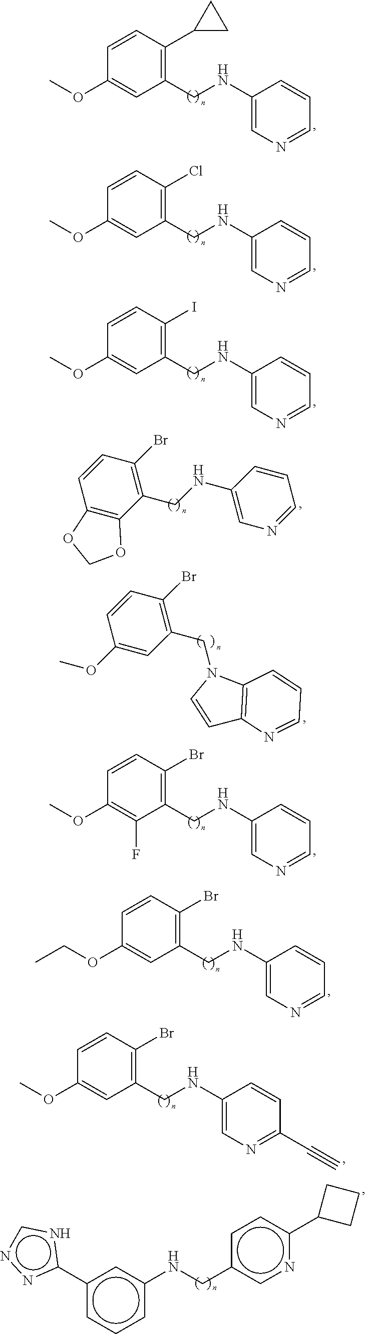

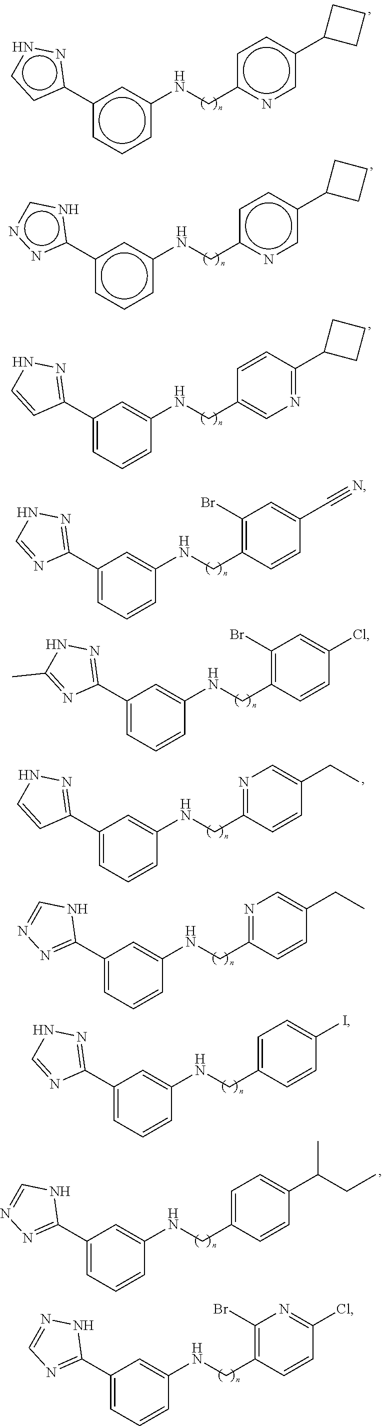

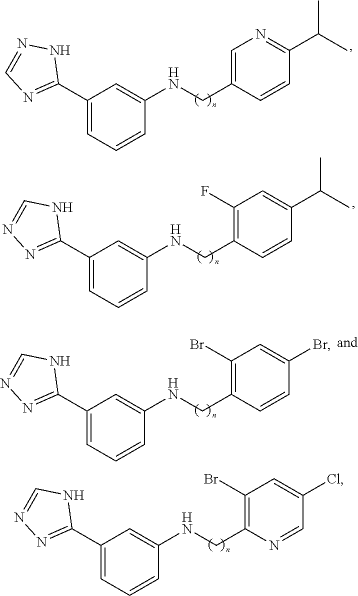

or a pharmaceutically acceptable salt thereof, wherein n=1.

Embodiment 51. The compound of embodiment 50, wherein the compound is:

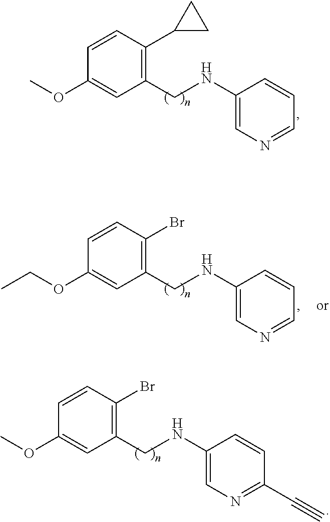

Embodiment 52. The compound of embodiment 50, wherein the compound is:

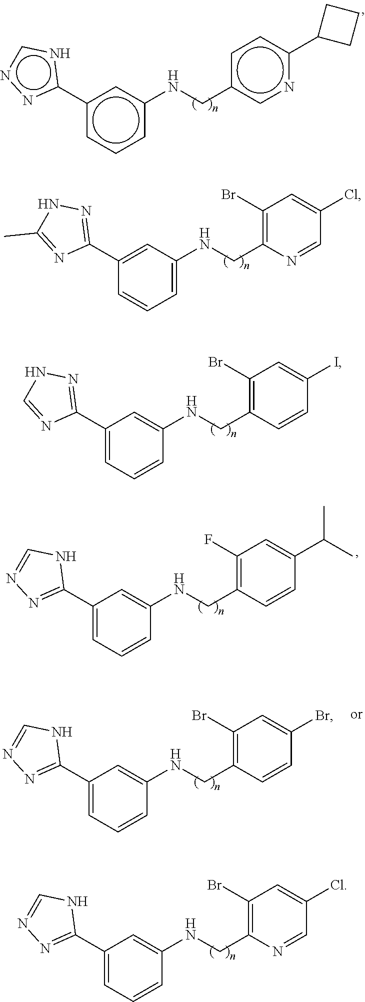

Embodiment 53. The compound of embodiment 51, wherein R² is attached at the 1 or 4 position of the phenyl ring.

Embodiment 54. A pharmaceutical composition comprising the compound of embodiment 50, and a pharmaceutically acceptable carrier.

### EXAMPLES

In embodiments, compounds described herein possess at least one property or characteristic that is of therapeutic relevance. Candidate agonists or inverse agonists may be identified by using, for example, an art-accepted assay or model. The skilled artisan is aware of other procedures, assay formats, and the like that can be employed to generate data and information useful to assess the MT1 and MT2 type-selective receptor modulators described herein.

After identification, candidate modulatoes can be further evaluated by using techniques that provide data regarding characteristics of the modulators (e.g., pharmacokinetic parameters). Comparisons of the candidate modulators to a reference standard (which may the “best-of-class” of current modulators) are indicative of the potential viability of such candidates. MT1 and MT2 type-selective receptor modulators that can serve as reference or benchmark compounds include those shown to demonstrate desired activity and characteristics useful for analyzing candidate modulators which will be apparent to the skilled artisan.

### Methods

### Molecular Docking

The MT₁ receptor bearing binding site mutations of G104^{3.29}→4 A104 and W251^{W6.48}→F251, as determined crystallographically, was used in the docking calculations. To prepare the structure for docking, atoms of the co-crystallized ligand, 2-phenylmelatonin, were used to seed the matching sphere calculation in the orthosteric site; these spheres represent favorable positions for individual ligand atoms to dock; overall 45 spheres were used. DOCK3.7 orients flexibases of pre-calculated ligand conformations into the orthosteric site by overlaying atoms of each library molecule onto these matching spheres. The receptor structure was protonated by REDUCE (Word J. et al., J Mol Biol 285, 1735-1747, 1999) and assigned AMBER united atom charges. For residues N162^{4.60} and Q181^{ECL2}, the dipole moment was increased without changing the net charge of the residues, as previously (Carlsson, J. et al. J Med Chem 53, 3748-3755, 2010). The radius of the low protein dielectric, which dictates the boundary between solute and solvent for Poisson-Boltzmann electrostatic calculations, was extended out 1.9 Å from the protein surface using spheres calculated by SPHGEN (Kuntz, I. D. et. al., J Mol Biol 161, 269-288, 1982). Scoring grids were pre-calculated by CHEMGRID (Meng, E. C. et al., Journal of Computational Chemistry 13, 505-524, 1992) for AMBER van der Waals potential, QNIFFT (Gallagher, K. & Sharp, K. Biophys J 75, 769-776, 1998) for Poisson-Boltzmann-based electrostatic potentials, and SOLVMAP (Mysinger, M. M. & Shoichet, B. K. J Chem Inf Model 50, 1561-1573, 2010) for ligand desolvation.

The resulting potential grids and ligand matching parameters were evaluated for their ability to enrich known MT₁ ligands over property-matched decoys (Mysinger, M. M. et al., J Med Chem 55, 6582-6594, 2012). Decoys share the same physical properties as known ligands but are topologically dissimilar and so unlikely to bind. Thirty-one known MT₁ melatonin receptor ligands, both agonists and antagonists, were extracted from the IUPHAR database (Southan, C. et al. Nucleic Acids Res 44, D1054-1068, 2016), and 1550 property-matched decoys were generated using the DUD-E pipeline (Mysinger, supra). Docking success was judged on the ability to enrich the known ligands over the property-matched decoys by docking score and rank, using adjusted logAUC (Mysinger, supra); this is widely done in the field. We also ensured that molecules with extreme physical properties were not enriched, as can happen when only counter-screening against property-matched decoys. In particular, we wanted to ensure that neutral molecules were enriched over charged ones. The docking parameters were also judged on how well they reproduced the known ligands' expected binding modes and their ability to hydrogen-bond with N162^{4.60} and Q181^{ECL2}.

The “lead-like” subset of ZINC15 (http://zinc15.docking.org) was then docked against the MT₁ orthosteric site, using DOCK3.7 (Coleman, R. G. et al., PLoS One 8, e75992, 2013). This library contained over 150,00,000 molecules, mostly make-on-demand from the Enamine REAL set (Lyu, J. et al. Nature 566, 224-229, 2019). Of the 150,000,000, over 135,000,000 molecules successfully docked. An average of 3445 orientations were calculated for each, and for each orientation, an average of 485 conformations were sampled. A simplex minimizer was used for rigid-body minimization on the best-scored pose for each ligand. Overall, about 72 trillion complexes were sampled and scored. The calculation time was 45,020 core hours, or 1.25 calendar days on 1500 cores.

To reduce redundancy of the best-ranking docked molecules, the top 300,000 ranked molecules were clustered by ECFP4-based Tanimoto coefficient (Tc) of 0.5, and the best-scoring member was used to represent the cluster. This produced 65,323 topologically diverse clusters, which were filtered for novelty by calculating ECFP4-based Tcs against over 1100 annotated MT₁ and MT₂ receptor ligands, extracted from the CHEMBL23 (Bento, A. P. et al. Nucleic Acids Res 42, D1083-1090, 2014). database. Molecules with Tc≥0.38 were considered too similar to known MT₁/MT₂ ligands and were not further pursued.

After filtering for novelty, the docked poses of the best-scoring members of each cluster were filtered by the proximity of their polar moieties, if any, to N162^{4.60} or Q181^{ECL2}, and manually inspected for favorable geometry and interactions. Of the best-scoring molecules so prioritized, all members of its cluster within the top 300,000 molecules were also inspected, and sometimes one of these was chosen if they exhibited more favorable poses or chemical properties. Ultimately, forty compounds were chosen for testing, thirty-eight of which were successfully synthesized (a 95% fulfillment rate). As far as we are know, none of these compounds has been previously available, and we are unaware of reports of them even being previously synthesized.

Make-On-Demand Synthesis

Compounds were synthesized using 72,000 qualified in stock building blocks and 130 well-characterized, two component reactions at Enamine. Historically, molecules have been synthesized in three to four weeks with an 85% fulfilment rate; in this project delivery time was six weeks, but with a 95% fulfilment rate for the 40 molecules prioritized from the initial docking screen. Each reaction is tested for conditions including temperatures, completion time, and mixing, as described⁵⁴. Typically, compounds are made in parallel by combining reagents and solvents in a single vial in the appropriate conditions to allow the reaction to proceed to completion. The product-containing vial is filtered by centrifugation into a second vial to remove precipitate and the solvent is evaporated under reduced pressure; the product is then purified by HPLC. Identity and purity is assessed by LC/MS and ¹H NMR. All compounds were shipped 95% pure or better, and the main three compounds '7447, '3384 and '4226 were independently confirmed to be ≥95% pure by LC/MS in secondary confirmation analyses (Data not shown). Details on synthesis and analyses may be found in the SI methods section 2.

Structure-Based Ligand Optimization

After experimental testing (below), 1₂ of the 15 actives from docking were prioritized for optimization, representing a range of activities and type selectivity. Several thousand analogs of these actives, each bearing the same scaffold as the parent molecule and with Tc<0.38 to annotated melatonin receptor ligands, were selected from the ZINC database and docked to the MT₁ binding site, again using DOCK3.7. The resulting docked poses were manually evaluated for interactions with N162^{4.60} or Q181^{ECL2} and 132 analogs were selected for de novo synthesis at Enamine, in two iterations of analoging. Of these, 131 were successfully synthesized, a >99% fulfillment rate.

In-Vitro Methods

Cell Culture

HEK-293 T cells were maintained with complete Dulbecco's modified Eagle's medium (DMEM), which is composed of 10% fetal bovine serum (FBS), 2 mM L-glutamine, 100 units/ml penicillin G, 100 μg/ml streptomycin at 37° C. in the presence of 5% CO₂

Tango Arrestin Recruitment Assay

MT₁ and MT₂ Tango constructs were designed and assays were performed as previously described (Kroeze, W. K. et al. Nat Struct Mol Biol 22, 362-369, 2015). HTLA cells stably expressing TEV protease fused 1₃-arrestin (kindly provided by Dr. Richard Axel) and tTA dependent luciferase reporter gene were transfected with MT₁ or MT₂ Tango construct. The next day, transfected cells were seeded into poly-L-lysine coated 384-well white clear bottom cell culture plates with DMEM containing 1% dialyzed FBS at a density of 20,000 cells per well in 40 μl for another 6 hour. Drug solution was prepared in the same media used for cell plating at 5× final concentration (10 μl per well) and added for overnight incubation. The next day, media and drug solutions were discarded and loaded with 20 μl per well of Bright-Glo reagent (Promega). Plates were incubated for 20 mins in the dark followed by being counting using SpectraMax luminescence reader (Molecular Device). Data were analyzed using GraphPad Prism 6.0.

cAMP Assay

MT₁ and MT₂ receptors were tested using Promega's spilt luciferase based GboSensor cAMP biosensor technology. HEK-T cells were plated in 15 cm cell culture dish (at a density of 18 million cells) containing DMEM composed of 10% dialyzed FBS, 2 mM L-glutamine, 100 units/ml penicillin G, 100 μg/ml streptomycin for 4-6 hour. Then, cells were co-transfected with 8 μg of construct which encodes either MT₁ or MT₂ (de-Tangonized constructs) and 8 ag of Glosensor DNA. Next day, transfected cells were seeded into poly-L-lysinecoated 384-well white clear bottom cell culture plates with complete DMEM with 1% dialized FBS at a density of 20,000 cells per well for another 24 h. The next day, cell medium were discarded and loaded with 20 μl of assay buffer (1×HBSS, 20 mM HEPES, pH 7.4, 0.1% BSA). To measure agonist activity of MT₁ or MT₂ receptor, 10 μl of test compound solution at 3× final concentration was added for 15 minutes followed by addition of 10 μl of luciferin/isoproterenol mixture (at a final concentration of 4 mM and 200 nM respectively) for another 15 mins for luminescence quantification. Then, plates were counted using SpectraMax luminescence reader (Molecular Device). Data were analyzed using GraphPad Prism 6.0.

Log(Eₘₐₓ/EC₅₀) calculation and ligand bias quantification The ΔLog(Eₘₐₓ/EC₅₀) was calculated with melatonin as a reference agonist for G protein and 8-arrestin pathway, and the ΔΔLog(Eₘₐₓ/EC₅₀) was calculated between two pathways for each ligand (Kenakin, T., Watson, C., Muniz-Medina, V., Christopoulos, A. & Novick, S. A simple method for quantifying functional selectivity and agonist bias. ACS Chem Neurosci 3, 193-203, doi:10.1021/cn200111m (2012). The bias factor is unitless and defined as 10ΔΔLog(Eₘₐₓ/EC₅₀). Corresponding bias plot was also generated (Kenakin, T. Pharmacol Rev 71, 267-315, 2019).

GPCR-Ome Counter-Screen

Screening of compounds in the PRESTO-Tango GPCR-ome was accomplished as previously described (Kroeze, supra). with several modifications. First, HTLA cells were plated in DMEM with 10% FBS and 10 U/mL penicillin-streptomycin. Next, the cells were transfected using an in-plate PEI method (Longo, P. A. et al., Methods Enzymol 529, 227-240, 2013). PRESTO-Tango receptor DNAs were resuspended in OptiMEM and hybridized with PEI prior to dilution and distribution to 384-well plates and subsequent addition to cells. After overnight incubation, drugs were added to cells without replacement of the medium. The remaining steps of the PRESTO-Tango protocol were followed as previously described.

Reagents and Ligands

2-[¹²⁵I]-Iodomelatonin (SA: 2,200 ci, 81.4 TBq/mmol) was purchased from Perkin Elmer (Shelton, Conn., USA). Guanosine 5′-triphosphate sodium salt hydrate (GTP), melatonin and all other chemicals and reagents were obtained from Sigma-Aldrich (St. Louis, Mo., USA).

Compound Preparation

For receptor binding studies, '7447 was dissolved in 50% DMSO/50% ethanol for 13 mM stock solution, diluted 1/10 in 100% ethanol then 1/10 again in 50% ethanol/50% Tris-HCl buffer. Both '3384 and '4226 were dissolved in 100% ethanol for 1₃ mM stock solutions and then diluted 1/10 in 50% ethanol/50% Tris-HCl buffer. Further dilutions were done in Tris-HCl buffer.

2-[¹²⁵I]-Iodomelatonin Competition Binding

CHO cells stably expressing FLAG tagged recombinant hMT₁ or hMT₂ melatonin receptors (mycoplasma free; authenticated by 2-[¹²⁵I]-Iodomelatonin saturation binding) were grown in culture as monolayers in Ham's F12 media supplemented with fetal calf serum (10%), penicillin (1%; 10,000 I.U/ml)/streptomycin (5%; 10,000 μg/ml) in CO₂ at 37° C. as described (Gerdin, M. J. et al., J Pharmacol Exp Ther 304, 931-939, 2003). Cells were grown for 4 days to 90-95% confluence, then washed with PBS (potassium phosphate buffer, 10 mM, pH 7.4), detached with PBS containing 0.25 M sucrose and 1 mM EDTA, and pelleted by centrifugation (1700×g, 5 min) as described (Popovska-Gorevski, M. et al., Chem Res Toxicol 30, 574-582, 2017). Cell pellets were suspended and homogenized in control buffer (50 mM Tris-HCl, 10 mM MgCl₂; pH 7.4 at 25° C.) and washed twice by centrifugation (17,000×g, 15 min) in control or inactive conformation buffer (50 mM Tris-HCl, 10 mM MgCl₂, 100 μM GTP, 1 mM EDTA.Na₂, 150 mMNaCl, pH 7.4 at 25° C.) as described (Popovska-Gorevski, M., supra). 2-[¹²⁵1]-Iodomelatonin binding affinity was determined on membranes from CHO-hMT₁ (9.6±0.3 μg protein/assay; Bₘₐₓ: 1154±38 fmol/mg protein, n=3) and CHO-hMT₂ (15±1 μg protein/assay; Bₘₐₓ: 352±19 fmol/mg protein, n=3) cells. Ligand competition (10 μM to 100 μM) for 2-[¹²⁵I]-iodomelatonin (104±2 μM, n=30) binding was performed in control or inactive conformation buffer in a total volume of 0.26 mL as described (Popovska-Gorevski, M., supra). Assays were incubated for 1 hour at 25° C. Bound radioligand was separated from freeby rapidvacuum filtration using glass microfiber filters (Whatman, Krackeler Scientific, Inc., Albany N.Y., USA) saturated in 0.5% polyethylenimine solution. Total radioactivity bound to the filters was determined on a gamma counter (Popovska-Gorevski, M., supra).

Data Analysis

Kᵢ values were calculated from IC₅₀ values using GraphPad PRISM™ 8.0 according to the Cheng-Prusoff equation (Cheng, Y. & Prusoff, Biochem Pharmacol 22, 3099-3108 (1973). Kᵢ=IC₅₀/(1+[L]/K_{D}) where L is the concentration of radioligand, K_{D} is the dissociation constant of 2-[I¹²⁵I]-iodomelatonin in control or inactive conformation buffer for the hMT₁ (Control K_{D}=116 μM; Inactive K_{D}=280 μM) and hMT₂ (Control K_{D}=119 μM; Inactive K_{D}=215 μM) receptors. Affinity shifts induced by G protein inactivation were measured by subtracting pKᵢ(inactive) from pKᵢ(Control) (ΔpKᵢ) and normalization by melatonin ΔpKᵢ (CHO-hMT₁: 1.19; CHO-hMT₂: 0.41). Affinity shifts or lack thereof with G protein inactivation indicate apparent efficacy (Lefkowitz, R. J. et al., J Biol Chem 251, 4686-4692, 1976) as ligands are classified as agonists (ΔpKᵢ% MLT>20%), antagonists (ΔpKᵢ% MLT<20%, >−20%), or inverse agonists (ΔpKᵢ% MLT<−20%) accordingly.

In-Vivo Methods

Animals and Housing

Male and female C₃H/HeN (C₃H) wild-type (WT), MT₁ knockout (MT₁KO), and MT₂ knockout (MT₂KO) mice (average 6.28 months) used in this study were raised in our breeding colony at University at Buffalo. C₃H/HeN mice homozygous for the MT₁ and MT₂ melatonin receptor gene deletion and their WT controls were generated from breeding pairs donated by Dr. S. M. Reppert (University of Massachusetts Medical School, Worcester, Mass., USA) and backcrossed with C₃H mice [Harlan (now Envigo), Indianapolis, Ind., USA] for at least seven generation as described in detail (Sumaya, I. C. et al., J Pineal Res 39, 170-177, 2005). Genotype was confirmed using tail samples at the end of each experiment and was verified periodically during the tenure of the colony. The strains of mice in our breeding colony were re-derived periodically by backcrossing with WT mice to reduce genetic drift.

Mice were group housed (3-5 per cage) with corncob bedding in polycarbonate translucent cages (30×19 cm) and maintained in a 14/10 light-dark (LD) cycle (Zeitgeber time 0 or ZT0 corresponds to Lights ON and ZT 14 to Lights OFF) in temperature and humidity controlled rooms with ad libitum access to food and water in the Laboratory Animal Facility at the University at Buffalo. Light levels were 200-300 lux at the level of the cage. Treatments and animal care performed in the dark were under a dim red safelight (15 watts, Kodak 1A filter) with illuminance of less than 3-5 lux (Benloucif, S. & Dubocovich, M. L. J Biol Rhythms 11, 113-125, (1996). All experimental procedures using mice were conducted in accordance with guidelines set forth by the National Institutes of Health and approved by the University at Buffalo Institutional Animal Care and Use Committee.

Circadian Rhythm Measurement

Circadian rhythm phase was determined for each mouse using the onset of running wheel activity defined as CT12 (Circadian Time 12). Running wheel activity was measured continuously via magnetic microswitches detecting wheel revolutions with a computer equipped with Clocklab data collection Software™ (Actimetrics: Wilmette, Ill.). All actigraphy data was visualized and analyzed using ClockLab™ and MATLAB™ software. All mice were individually housed in cages (33×15 cm) equipped with running wheels in light-tight ventilated cabinets with controlled temperature and LD cycles (Phenome Technologies: Skokie, Ill.). Male and female mice were housed in separate cabinets for all experiments.

Phase Shift

Changes in circadian phase induced by vehicle or drugs administered at various circadian times was assessed in WT, MT₁KO, and MT₂KO male and female C3H mice (3 to 8 months) using methods and protocols previously described (Dubocovich, M. L. et al., FASEB J 12, 1211-1220, 1998; Benloucif, supra). Following a period of 14 days in a LD cycle mice were placed in constant dark (DD) beginning at ZT12 (dark onset). Mice were keptin DD (2-3 weeks) untilastable free-running phase of running wheel activity rhythm onset was established. Circadian times of treatment were predicated from best fit lines of running wheel activity onsets for of running either pre (7-14 days) and post (7-14 days) treatment. Treatment times were at CT10 (CT9-11) and CT2 (CT1-3) within a 3 hour window. Mice were treated (0.1 ml/mouse, s.c.) with vehicle (30% ethanol saline, s.c.) or drugs (melatonin, '3384, '7447, '4226 at 0.9 μg and 30 μg/mouse in vehicle) for 3 consecutive days at the appropriate circadian time under dim red light. Vehicle or drug treatments were repeated for 3 consecutive days at the selected circadian time following the three-pulse treatment protocol described (Benloucif, supra). Phase shifts were quantified using the best-fit lines for onsets of activity during pre and post treatment periods. Differences are characterized as phase delays (pre-treatment ahead of post treatment best fit line onset) or phase advances (post treatment ahead of pre treatment best fit line onset) of running wheel activity onset rhythms.

Re-Entrainment Experiments

Male and female C3H WT, MT₁KO, and MT₂KO mice (3 to 6 months) were maintained under a 12:12 LD cycle for at least 2 weeks prior experimental manipulations to allow stable entrainment to dark onset before advance of the LD cycle. Actigraphy data was recorded as described above and all experiment protocols performed as described (Dubocovich, M. L. et al., J Pineal Res 39, 113-120, 2005). On the first day of treatment, the dark onset was advanced 6 h. This resulted in a short night and mice were treated (0.1 ml/mouse s.c.) with vehicle (30% ethanol saline, s.c.) or drugs (melatonin, '3384 or '7447 at 30 ug/mouse in vehicle) for 3 consecutive days 30 minutes prior to the new dark onset. Post treatment, mice were given 14-20 days to re-entrain running wheel activity onsets to the new dark onset. Using exported running wheel activity onsets from actograms, onset hours advanced each day were determined by subtracting this value each day from the average onset of running wheel activity for 3 days prior to treatment for each mouse. Further, using the data from this calculation combined with visualization of actograms, the number of days to reach stable re-entrainment was determined for each mouse.

Data Analyses

All statistical analyses as described in further detail for each experiment were conducted using GraphPad Prism 8™ (La Jolla, Calif.). All data sets were visualized for normality using QQ plots of predicted vs. actual residuals. Actigraphy data was generated for visualization blind to treatment prior to the quantification and statistical analysis stages. For phase shift and re-entrainment experiments we determined statistical power a-priori (a error probability=0.05) based on data for a known effect size for melatonin in these paradigms (G-power 3.0.10) (Dubocovich, M. L. et al., FASEB J 12, 1211-1220, 1998; Dubocovich, M. L. et al., J Pineal Res 39, 113-120, 2005). Group comparisons for phase shift in FIG. 4E were made by one-way ANOVA (P<0.05) comparing hours shifted of circadian running wheel activity rhythm onsets among all 5 groups (vehicle, melatonin, '7447, '3384, '4226) accompanied with post-hoc analyses by Dunnet's to determine individual group differences compared to vehicle (P<0.05). Comparisons for phase shift in FIG. 10G were also made by one-way ANOVA (P<0.05) comparing shifts of circadian running wheel activity rhythm onsets among all 4 groups (vehicle, melatonin, '7447, '4226). Data in FIGS. 4F & 4G were compared via a two-way ANOVA (3×2: genotype x treatment) with Tukey's post hoc analyses (P<0.05). Comparisons for FIG. 4L, FIGS. 11H-11J were made by mixed effect two-way repeated measures ANOVA (treatment×time) with Sidak's post hoc test (P<0.05). Number of days to re-entrainment was compared via one-way ANOVA or two-way ANOVA for FIG. 4M & FIG. 4N with a Dunnet's & Tukey's post hoc test (P<0.05) respectively. P values and values for statistical analyses are included in figure legends. No sex differences in treatment effects were evident in any data set when assessed via two-way ANOVA or three-way ANOVA where appropriate; therefore, data were pooled between male and female mice for analyses described. The n values represent the number of individual mice per condition in each experiment.

### Example 1. Synthetic Routes for MT₁/MT₂ Compounds

All chemicals and solvents for the synthesis of compounds were obtained from Enamine and used without further purification. ¹H and ¹³C NMR spectra were acquired on Bruker Advance DRX 400 and Bruker Avance DRX 500 spectrometers using DMSO-d₆ or Chloroform-dl as a solvent and tetramethylsilane as an internal standard. LC/MS data were recorded on Agilent 1100 HPLC equipped with diode-matrix and mass-selective detector Agilent LC/MSD SL, column: Zorbax SBC18, 4.6 mm×15 mm; eluent, A, acetonitrile-water with 0.1% of TFA (95:5), B, water with 0.1% of TFA; flow rate: 1.8 mL/min. Crude samples with product content below 90% were purified using mass-triggered Agilent 1200 HPLC systems utilizing various gradients depending on a S log P value of a particular compound. Purity of compounds were assessed based on ¹H NMR and LC/MS data. Optical rotation values were measured with a JASCO J-20 polarimeter with a 50 mm cell at 25° C. at 589 nm (sodium D-line). [α]_{D}²⁵ values are given in 10⁻¹degcm²g⁻¹.

Method 1. An amine (100 mg), DIPEA (1.2 mol equivalent to the amine) and DMSO (0.5 mL) were placed into a 4 mL capped glass vial and stirred for 30 min. After addition of an alkyl halide (1.2 mol. eq. to the amine), the vial was stirred for 1 hour at rt. Then the vial was placed into a thermostat (set to 100° C.) for 9 hours. After cooling down the mixture was filtered; the solvent and volatile components were evaporated under reduced pressure to give the crude product. The product was further purified by HPLC.

Method 2. An amine (100 mg), acid (1.2 mol equivalent to the amine), DIPEA (1.2 mol equivalent to the amine) and acetonitrile (0.5 mL) were placed into a 4 mL capped glass vial and stirred for 30 min. After addition of 2-chloro-1-methylpyridin-1-ium iodide (1.44 mol equivalent to the amine), the vial was stirred for 1 hour at rt. Then the vial was placed into a thermostat (set to 100° C.) for 6 hours. After cooling down the mixture was filtered; the solvent and volatile components were evaporated under reduced pressure to give the crude product. The product was further purified by HPLC.

Method 3. An amine (100 mg), an acid (1.1 mol. eq. to the amine) and 0.5 mL of DMSO were placed into a 4 mL capped glass vial and the mixture was stirred for 30 min. Then EDC (1.2 mol. eq. to the amine) was added and the mixture was stirred for 1 hour. If the solution was transparent, the mixture was left overnight at room temperature as is; otherwise, the vial was placed in the ultrasonic bath and left overnight. The solution was filtered, and the solvent and volatile components were evaporated under reduced pressure to give the crude product. The product was further purified by HPLC.

Method 4. An amine (100 mg), DIPEA (1.2 mol equivalent to the amine) and DMSO (0.5 mL) were placed into a 4 mL capped glass vial and stirred for 30 min. After addition of an aryl halide (1 mol equivalent to the amine), was stirred for 1 hour at rt. Then the vial was placed into a thermostat (set to 100° C.) for 9 hours. After cooling down the mixture was filtered; the solvent and volatile components were evaporated under reduced pressure to give the crude product. The product was further purified by HPLC.

Method 5. An amine (100 mg), alkyl halide (1 mol equivalent to the amine) and DMSO (0.5 mL) were placed into a 4 mL capped glass vial and stirred for 30 min. After addition of 4M methanol solution of KOH (5 mol equivalent to the amine), the vial was stirred for 1 hour at rt. Then the vial was placed into a thermostat (set to 100° C.) for 8 hours. After cooling the mixture, KOH was neutralized with gaseous CO2 (stirred for 6 hours at rt). The solution was filtered, and the solvent and volatile components were evaporated under reduced pressure to give the crude product. The product was further purified by HPLC.

Method 6. An amine (100 mg), TEA (1.2 mol equivalent to the amine) and acetonitrile (0.5 mL) were placed into a 4 mL capped glass vial and stirred for 30 min. After addition of sulfonyl halide (1 mol equivalent to the amine), the solution was stirred for 12 hours at rt. The solution was filtered, and the solvent and volatile components were evaporated under reduced pressure to give the crude product. The product was further purified by HPLC.

Method 7. An amine (100 mg), DMF (0.5 mL) and an aldehyde (1 mol. eq. to the amine) were placed into a 4 mL capped glass vial and stirred for 5 hours in a thermostat (set to 100° C.). After cooling down to rt, methanol (1 mL) and NaBH4 (70 mg) were added; the vial was then placed into the ultrasonic bath for 2 hours and left stirring at rt for 12 hours. Then methanol (1 mL) was added, and the vial was again placed in the ultrasonic bath for 3 hours. After cooling down to the mixture, chloroform (3 mL) and water (5 mL) were added, the vial was left sharing for 15 min, and the aqueous layer was removed. The solvents and volatile components were evaporated under reduced pressure to give the crude product. The product was further purified by HPLC.

Method 8. An amine (100 mg), DIPEA (1.2 mol equivalent to the amine) and DMSO (0.5 mL) were placed into a 4 mL capped glass vial and stirred for 30 min. After addition of an alkyl halide (1.2 mol. eq. to the amine), the vial was stirred for 12 hours at rt. Then the vial was placed into a thermostat (set to 100° C.) for 9 hours. After cooling down the mixture was filtered; the solvent and volatile components were evaporated under reduced pressure to give the crude product. The product was further purified by HPLC.

Method 9. An amine (100 mg), a ketone/an aldehyde (1 mol. eq. to the amine) and chloroform (3 mL) were placed into a 4 mL capped glass vial and left shaking for 30 min at rt. To this mixture NaBH(OAc)₃ (2 mol. eq. to the amine) was then added. The vial was left shaking at rt for 24 hours, and then at 45° C. for 48 hours. After cooling the mixture, ammonia solution (3-4 mL) was added and the vial was stirred for 15 min. The aqueous layer was removed, and chloroform was evaporated under reduced pressure to give the crude product. The product was further purified by HPLC.

Method 10. An amine (100 mg), alkyl halide (1 mol equivalent to the amine) and DMSO (0.5 mL) were placed into a 4 mL capped glass vial and stirred for 30 min. After addition of 4M methanol solution of KOH (5 mol equivalent to the amine), the vial was stirred for 1 hour at rt. Then the vial was placed into a thermostat (set to 100° C.) for 8 hours. After cooling the mixture chloroform (3 mL) and water (0.5 mL) were added to it, the vial was left shaking for 15 minutes, and then water layer was removed. The chloroform was evaporated under reduced pressure, and 0.6 mL of trifluoroacetic acid was added to the residue. The vial was left shaking for 12 hours at rt, then 3 mL of chloroform was added into the vial, and all the solvent and volatile components were evaporated under reduced pressure to give the crude product. The product was further purified by HPLC.

### N-(benzo[d][1,3]dioxol-5-ylmethyl)-2-chloro-N-methylpyridin-4-amine (Method 4)

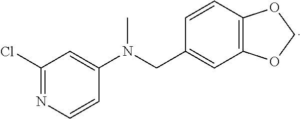 [SMILES: CN(Cc1ccc2c(c1)OCO2)c1ccnc(Cl)c1]

Yield: 39%. 1H NMR (500 MHz, DMSO-d₆) δ 7.88 (d, J=5.9 Hz, 1H), 6.85 (d, J=7.8 Hz, 1H), 6.75 (s, 1H), 6.68-6.60 (m, 3H), 5.98 (s, 2H), 4.54 (s, 2H), 3.03 (s, 3H). ¹³C NMR (126 MHz, DMSO-d₆) δ 155.9, 151.8, 149.4, 148.0, 146.8, 131.6, 120.2, 108.8, 107.6, 107.2, 105.7, 101.4, 54.4, 38.5. LC/MS (APSI) m/z [M+H] calculated for C₁₄H₁₄ClN₂O₂: 276.1; found: 276.7.

### 5-(((5-cyclopropyl-4H-1,2,4-triazol-3-yl)thio)methyl)thiophene-2-carboxylate (Method 1)

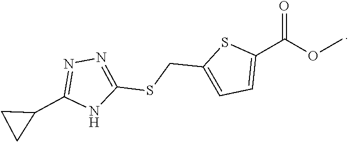 [SMILES: COC(=O)c1ccc(CSc2nnc(C3CC3)[nH]2)s1]

Yield: 67%. ¹H NMR (500 MHz, DMSO-d₆) δ 7.60 (d, J=3.9 Hz, 1H), 7.08 (d, J=3.8 Hz, 1H), 4.53 (s, 2H), 3.77 (s, 3H), 1.99 (tt, J=8.7, 4.7 Hz, 1H), 1.00 (dq, J=7.0, 3.7 Hz, 2H), 0.87 (p, J=4.0 Hz, 2H). ¹³C NMR (126 MHz, Chloroform-d) δ 162.2, 150.3, 133.8, 131.9, 128.3, 52.6, 39.5, 8.6, 7.7. LC/MS (APSI) m/z [M+H] calculated for C₁₂H₁₄N₃O₂S₂: 296.1; found: 296.0.

### 3-(difluoromethoxy)-N-(3-hydroxy-4-methoxybenzyl)-N-methylbenzamide (Method 3)

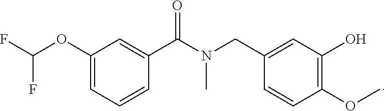 [SMILES: COc1ccc(CN(C)C(=O)c2cccc(OC(F)F)c2)cc1O]

Yield: 60%. ¹H NMR (500 MHz, DMSO-d₆) δ 9.03 (s, 1H), 7.62-6.97 (m, 5H), 6.97-6.62 (m, 2H), 6.76-6.44 (m, 1H), 4.51 (s, 1H), 3.74 (d, J=5.3 Hz, 3H), 2.76 (s, 2H). ¹³C NMR (126 MHz, DMSO-d₆) δ 169.4, 151.3 (t, J=3.4 Hz), 147.5, 147.2, 138.8, 132.6-127.8 (m), 121.8-111.1 (m), 56.1, 49.8, 33.0. LC/MS (APSI) m/z [M+H] calculated for C₁₇H₁₈F₂NO₄: 338.1; found: 338.0.

### N-(4-methoxybenzyl)-3-(4H-1,2,4-triazol-3-yl)aniline (Method 7)

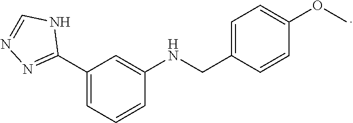 [SMILES: COc1ccc(CNc2cccc(-c3nnc[nH]3)c2)cc1]

Yield: 19%. ¹H NMR (500 MHz, DMSO-d₆) δ 14.00 (s, 1H), 8.49 (s, 1H), 8.00 (s, 1H), 7.28 (t, J=8.2 Hz, 3H), 7.15 (dt, J=17.5, 7.9 Hz, 2H), 6.88 (d, J=8.1 Hz, 2H), 6.63 (s, 1H), 6.36 (s, 1H), 4.24 (t, J=6.3 Hz, 2H), 3.71 (s, 2H). ¹³C NMR (126 MHz, DMSO-d₆) δ 158.6, 128.9, 127.7, 126.7, 114.2, 114.1, 55.5, 46.3. LC/MS (APSI) m/z [M+H] calculated for C₁₆H₁₇N₄O: 281.1; found: 281.0.

### N-(4-isopropylbenzyl)-3-(4H-1,2,4-triazol-3-yl)aniline (Method 7)

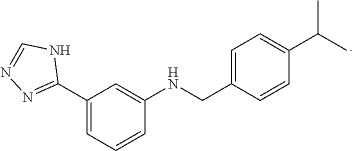 [SMILES: CC(C)c1ccc(CNc2cccc(-c3nnc[nH]3)c2)cc1]

Yield: 34%. ¹H NMR (500 MHz, DMSO-d₆) δ 8.25 (s, 1H), 7.29 (d, J=7.4 Hz, 3H), 7.21-7.15 (m, 3H), 7.12 (t, J=7.8 Hz, 1H), 6.63 (dd, J=7.9, 2.4 Hz, 1H), 6.37 (t, J=6.1 Hz, 1H), 4.25 (d, J=5.9 Hz, 2H), 2.84 (p, J=6.9 Hz, 1H), 1.17 (d, J=6.9 Hz, 6H). ¹³C NMR (126 MHz, DMSO-d₆) δ 149.5, 147.2, 137.9, 129.7, 127.72, 126.7, 114.0, 113.8, 110.1, 46.6, 33.6, 24.4.

LC/MS (APSI) m/z [M+H] calculated for C₁₈H₂₁N₄: 293.2; found: 293.2.

### N-(2-bromo-4-chlorobenzyl)-3-(4H-1,2,4-triazol-3-yl)aniline (Method 7)

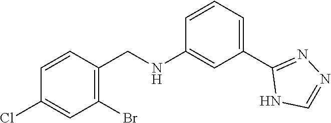 [SMILES: Clc1ccc(CNc2cccc(-c3nnc[nH]3)c2)c(Br)c1]

Yield: 27%. ¹H NMR (500 MHz, DMSO-d₆) δ 8.28 (s, 1H), 7.76 (d, J=2.1 Hz, 1H), 7.46-7.40 (m, 1H), 7.37 (d, J=8.4 Hz, 1H), 7.22 (d, J=5.7 Hz, 2H), 7.16 (t, J=7.9 Hz, 1H), 6.57 (t, J=7.7 Hz, 2H), 4.32 (d, J=5.9 Hz, 2H). ¹³C NMR (126 MHz, Chloroform-d) δ 148.8, 138.1, 132.6, 132.1, 130.9, 130.4, 129.9, 128.3, 123.5, 114.6, 113.7, 46.8. LC/MS (APSI) m/z [M+H] calculated for C₁₅H₁₃BrClN₄: 363.0; found: 363.0.

### 4-chloro-2-(((6-chloropyridin-3-yl)methyl)(methyl)amino)thiazole-5-carboxylate (Method 4)

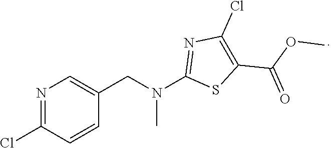 [SMILES: COC(=O)c1sc(N(C)Cc2ccc(Cl)nc2)nc1Cl]

Yield: 59%. 1H NMR (500 MHz, DMSO-d₆) δ 8.38 (d, J=2.5 Hz, 1H), 7.79 (dd, J=8.4, 2.6 Hz, 1H), 7.51 (d, J=8.3 Hz, 1H), 4.78 (s, 2H), 3.73 (s, 3H), 3.10 (s, 3H). ¹³C NMR (126 MHz, DMSO-d₆) δ 169.7, 160.5, 150.1, 149.7, 143.0, 139.6, 131.8, 124.9, 107.1, 52.4, 38.8. LC/MS (APSI) m/z [M+H] calculated for C₁₂H₁₂C₁₂N₃O₂S: 332.0; found: 332.0.

### N-(2-bromo-5-methoxybenzyl)pyridin-3-amine (Method 7)

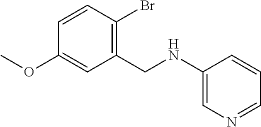 [SMILES: COc1ccc(Br)c(CNc2cccnc2)c1]

Yield: 41%. ¹H NMR (500 MHz, DMSO-d₆) δ 7.96 (d, J=2.8 Hz, 1H), 7.77 (d, J=4.6 Hz, 1H), 7.52 (d, J=8.8 Hz, 1H), 7.05 (dd, J=8.3, 4.6 Hz, 1H), 6.95 (d, J=3.0 Hz, 1H), 6.82 (td, J=8.6, 3.0 Hz, 2H), 6.51 (t, J=6.2 Hz, 1H), 4.27 (d, J=5.9 Hz, 2H), 3.67 (s, 3H). ¹³C NMR (126 MHz, Chloroform-d) δ 159.4, 144.7, 139.4, 137.9, 136.0, 133.7, 124.1, 118.2, 115.6, 114.5, 113.3, 55.8, 46.9. LC/MS (APSI) m/z [M+H] calculated for C₁₃H₁₄BrN₂O: 295.0; found: 295.0.

### (6-fluoropyridin-3-yl)(5-methoxy-3,4-dihydroisoquinolin-2(1H)-yl)methanone (Method 3)

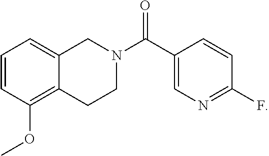 [SMILES: COc1cccc2c1CCN(C(=O)c1ccc(F)nc1)C2]

Yield: 67%. ¹H NMR (500 MHz, DMSO-d₆) δ 8.36 (d, J=8.5 Hz, 1H), 8.10 (s, 1H), 7.33-7.22 (m, 2H), 7.19 (s, OH), 7.14 (s, 1H), 6.83 (d, J=8.3 Hz, 2H), 4.75 (s, 1H), 3.90-3.79 (m, 1H), 3.77 (s, 3H), 3.55 (s, 1H), 2.72 (t, J=6.1 Hz, 2H). ¹³C NMR (126 MHz, Chloroform-d) δ 166.2-161.0 (m), 157.8 (d, J=147.8 Hz), 134.4, 130.9 (d, J=4.4 Hz), 127.5, 123.0, 119.0, 118.6, 111.7-107.9 (m), 108.7, 49.4, 45.0, 40.3 (d, J=9.5 Hz), 23.7. LC/MS (APSI) m/z [M+H] calculated for C₁₆H₁₆FN₂O₂: 287.1; found: 287.0.

### (1S,2S)—N-(4-methoxybenzyl)-2-methyl-N-(pyridin-4-yl)cyclopropanecarboxamide (Method 2)

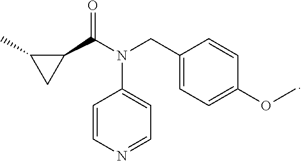

Yield: 46%. ¹H NMR (500 MHz, DMSO-d₆) δ 8.57-8.51 (m, 2H), 7.31-7.25 (m, 2H), 7.07 (d, J=8.1 Hz, 2H), 6.85-6.79 (m, 2H), 4.96 (d, J=15.4 Hz, 1H), 4.90 (d, J=15.4 Hz, 1H), 3.69 (s, 3H), 1.33-1.20 (m, 2H), 1.14 (dt, J=8.4, 3.9 Hz, 1H), 0.95 (d, J=5.9 Hz, 3H), 0.62-0.54 (m, 1H). ¹³C NMR (126 MHz, DMSO-d₆) δ 172.6, 158.8, 151.2, 150.0, 129.7, 129.3, 122.4, 114.3, 55.5, 50.9, 22.0, 17.8, 17.5, 16.9. LC/MS (APSI) m/z [M+H] calculated for C₁₈H₂₁N₂O₂: 297.2; found: 297.2.

### 7-(((5-(1H-pyrazol-5-yl)furan-2-yl)methyl)amino)-3-methylbenzo[d]oxazol-2(3H)-one (Method 7)

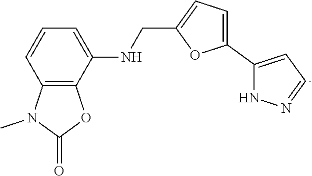 [SMILES: Cn1c(=O)oc2c(NCc3ccc(-c4ccn[nH]4)o3)cccc21]

Yield: 25%. ¹H NMR (500 MHz, DMSO-d₆) δ 12.88 (s, 1H), 7.74 (s, 1H), 6.97 (t, J=8.0 Hz, 1H), 6.62 (s, OH), 6.56 (d, J=8.4 Hz, 2H), 6.49 (d, J=7.7 Hz, 1H), 6.43 (d, J=2.2 Hz, 1H), 6.32 (d, J=5.8 Hz, 2H), 4.41 (d, J=6.1 Hz, 2H), 3.27 (s, 2H). ¹³C NMR (126 MHz, DMSO-d₆) δ 154.5, 132.5, 129.3, 124.8, 109.1, 107.2, 98.0, 28.5. LC/MS (APSI) m/z [M+H] calculated for C₁₆H₁₅N₄O₃: 311.1; found: 311.2.

### 2-methoxy-4-((6-methoxy-2H-benzo[b][1,4]oxazin-4(3H)-yl)methyl)benzonitrile (Method 9)

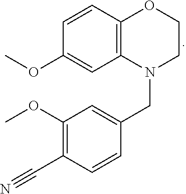 [SMILES: COc1ccc2c(c1)N(Cc1ccc(C#N)c(OC)c1)CCO2]

Yield: 21%. ¹H NMR (500 MHz, DMSO-d₆) δ 7.67 (dd, J=7.9, 1.6 Hz, 1H), 7.17 (s, 1H), 6.98 (d, J=7.9 Hz, 1H), 6.60 (dd, J=8.4, 1.6 Hz, 1H), 6.10 (dq, J=11.1, 2.3 Hz, 2H), 4.52 (s, 2H), 4.16 (t, J=4.3 Hz, 2H), 3.91-3.86 (m, 3H), 3.58-3.54 (m, 3H), 3.43-3.37 (m, 2H). ¹³C NMR (126 MHz, DMSO-d₆) δ 161.5, 154.6, 147.7, 138.2, 136.0, 134.3, 119.8, 116.9, 116.4, 111.1, 101.6, 99.5, 99.3, 64.4, 56.7, 55.6, 54.5, 48.0. LC/MS (APSI) m/z [M+H] calculated for C₁₈H₁₉N₂O₃: 311.1; found: 311.2.

### N-(benzo[d][1,3]dioxol-5-ylmethyl)-N-(2-methoxyethyl)-3-(trifluoromethyl)-1,2,4-thiadiazol-5-amine (Method 4)

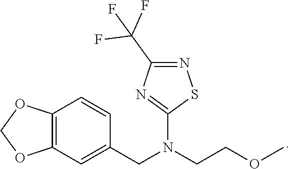 [SMILES: COCCN(Cc1ccc2c(c1)OCO2)c1nc(C(F)(F)F)ns1]

Yield: 61%. ¹H NMR (500 MHz, DMSO-d₆) δ 6.94-6.87 (m, 2H), 6.84 (d, J=8.0 Hz, 1H), 6.01 (s, 2H), 4.67 (s, 2H), 3.58 (t, J=5.1 Hz, 2H), 3.31 (s, 2H), 3.25 (s, 3H). ¹³C NMR (126 MHz, DMSO-d₆) δ 148.1, 147.5, 121.9, 108.8, 108.5, 101.6, 69.3, 58.6. LC/MS (APSI) m/z [M+H] calculated for C₁₄H₁₅F₃N₃O₃S: 362.1; found: 362.0.

### 4-iodo-3-((2-methylpyrimidin-4-yl)oxy)benzoate (Method 4)

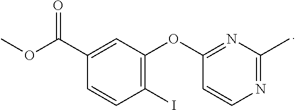

Yield: 28%. ¹H NMR (500 MHz, DMSO-d₆) δ 8.61 (d, J=5.8 Hz, 1H), 8.11 (d, J=8.2 Hz, 1H), 7.73 (s, 1H), 7.62 (d, J=8.3 Hz, 1H), 7.01 (d, J=5.8 Hz, 1H), 3.85 (s, 3H), 2.40 (s, 3H). ¹³C NMR (126 MHz, DMSO-d₆) δ 168.2, 168.1, 165.7, 160.0, 153.1, 140.7, 131.9, 128.3, 123.9, 106.0, 99.5, 53.0, 26.0. LC/MS (APSI) m/z [M+H] calculated for C₁₃H₁₂IN₂O₃: 371.0; found: 370.1.

### 5-methyl-4-((7-methyl-5-oxoimidazo[1,2-a]pyrimidin-1(5H)-yl)methyl)thiophene-2-carboxylate (Method 5)

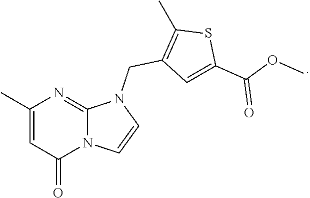

Yield: 34%. ¹H NMR (500 MHz, DMSO-d₆) δ 7.71 (d, J=2.3 Hz, 1H), 7.67 (t, J=2.5 Hz, 1H), 7.58 (t, J=2.5 Hz, 1H), 5.76 (d, J=2.3 Hz, 1H), 5.18 (d, J=2.5 Hz, 2H), 3.76 (d, J=2.4 Hz, 3H), 2.60 (d, J=2.4 Hz, 3H), 2.27 (d, J=2.4 Hz, 3H). ¹³C NMR (126 MHz, DMSO-d₆) δ 164.0, 162.0, 157.0, 146.3, 146.1, 135.4, 134.2, 129.2, 119.9, 106.7, 97.9, 52.6, 41.3, 24.6, 13.9.

LC/MS (APSI) m/z [M+H] calculated for C₁₅H₁₆N₃O₃S: 318.1; found: 318.0.

### 3-(((4-ethyl-5-methyl-6-oxo-1,6-dihydropyrimidin-2-yl)thio)methyl)benzoate (Method 1)

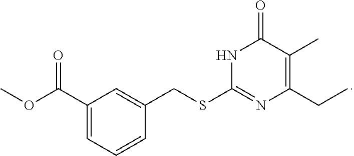 [SMILES: CCc1nc(SCc2cccc(C(=O)OC)c2)[nH]c(=O)c1C]

Yield: 55%. ¹H NMR (500 MHz, DMSO-d₆) δ 12.46 (s, 1H), 8.06 (s, 1H), 7.82 (d, J=7.7 Hz, 1H), 7.69 (d, J=7.7 Hz, 1H), 7.45 (t, J=7.7 Hz, 1H), 4.43 (s, 2H), 3.83 (s, 3H), 2.56-2.47 (m, 2H), 1.86 (s, 3H), 1.13 (t, J=7.5 Hz, 3H). ¹³C NMR (126 MHz, DMSO-d₆) δ 166.5, 139.6, 134.3, 130.2, 130.1, 129.2, 128.3, 52.6, 27.7, 12.4, 10.3. LC/MS (APSI) m/z [M+H] calculated for C₁₆H₁₉N₂O₃S: 319.1; found: 319.0.

### 1-(2-(2,2-difluorobenzo[d][1,3]dioxol-5-yl)ethyl)-7-methylimidazo[1,2-a]pyrimidin-5(1H)-one (Method 5)

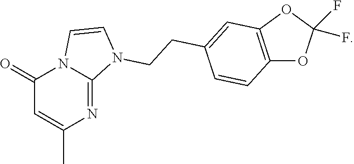 [SMILES: Cc1cc(=O)n2ccn(CCc3ccc4c(c3)OC(F)(F)O4)c2n1]

Yield: 25%. ¹H NMR (500 MHz, DMSO-d₆) δ 7.52 (d, J=2.5 Hz, 1H), 7.43 (d, J=2.6 Hz, 1H), 7.30 (s, 1H), 7.23 (d, J=8.1 Hz, 1H), 6.90 (d, J=7.9 Hz, 1H), 5.70 (s, 1H), 4.31 (t, J=6.9 Hz, 2H), 3.11 (t, J=6.9 Hz, 2H), 2.21 (s, 3H). ¹³C NMR (126 MHz, DMSO-d₆) δ 163.9, 157.0, 148.2-140.2 (m), 135.3, 125.1, 120.1, 111.0, 110.2, 106.2, 97.5, 46.0, 34.7, 24.5. LC/MS (APSI) m/z [M+H] calculated for C₁₆H₁₄F₂N₃O₃: 334.1; found: 334.0.

### N-(benzo[d][1,3]dioxol-5-ylmethyl)-N-(2-methoxyethyl)-3-(trifluoromethyl)-1,2,4-thiadiazol-5-amine (Method 4)

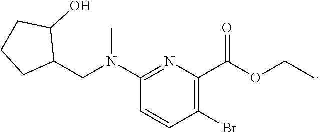 [SMILES: CCOC(=O)c1nc(N(C)CC2CCCC2O)ccc1Br]

Yield: 29%. ¹H NMR (500 MHz, DMSO-d₆) δ 7.70 (d, J=8.9 Hz, 1H), 6.72 (d, J=9.2 Hz, 1H), 4.43 (d, J=3.7 Hz, 1H), 4.30 (q, J=7.0 Hz, 2H), 3.93-3.88 (m, 1H), 3.58 (dd, J=14.3, 8.1 Hz, 1H), 3.43 (dd, J=14.3, 6.2 Hz, 1H), 3.00 (s, 3H), 2.01-1.92 (m, 1H), 1.70 (s, 1H), 1.71-1.61 (m, 1H), 1.61-1.52 (m, 2H), 1.48 (q, J=10.1, 8.8 Hz, 1H), 1.39 (s, 1H), 1.29 (t, J=7.1 Hz, 3H). ¹³C NMR (126 MHz, DMSO-d₆) δ 165.9, 156.9, 147.9, 142.0, 110.2, 101.2, 72.0, 61.8, 49.7, 44.5, 36.9, 34.8, 27.0, 21.7, 14.4. LC/MS (APSI) m/z [M+H] calculated for C₁₅H₂₂BrN₂O₃: 357.1; found: 357.0.

### Example 2. Effect of MT₁ Receptor Inverse Agonists and MT₂ Receptor Agonists on Circadian Rhythms and Re-Entrainment in Mice

We investigated a panel of widely-used MTR ligands, finding few with MT₁ type selectivity. Although reagents like N-acetyl tryptamine and 4P-PDOT were 39- and 150-fold MT₂-selective agonists, drugs like ramelteon, agomelatine, and tool compounds such as 8M-PDOT, had little type selectivity. For DH97 and Luzindole, which were previously reported as antagonists (Dubocovich, M. L. et al. Pharmacol Rev 62, 343-380, 2010), we observed partial agonism at the MT₂ receptor. Taken together with other studies (Liu, J. et al. Annu Rev Pharmacol Toxicol 56, 361-383, 2016; Comai, S. et al. Pharmacol Res 144, 343-356, 2019; Gobbi, G. & Comai, S., Front Endocrinol (Lausanne) 10, 87, 2019), there appears to be a lack of selective MT₁ ligands available to study the role of the MT₁ in vivo.

The determination of the MT₁ and MT₂ receptor crystal structures (Stauch, B. et al. Nature 569, 284-288, 2019; Johansson, L. C. et al. Nature 569, 289-292, 2019) afforded the opportunity to seek MT₁-selective ligands by docking an ultra-large make-on-demand library (Lyu, J. et al. Nature 566, 224-229, 2019). Given the similar MT₁ and MT₂ orthosteric sites (22 of 24 residues are identical), and the challenges of recognizing selective molecules in large library docking (Weiss, D. R. et al. J Med Chem 61, 6830-6845, 2018), we prioritized new, high-ranking chemotypes from the docking screen, unrelated to known melatonin receptor ligands, as these might interact differentially with the MTR types.

Over 150 million molecules with favorable physical properties (e.g., cLopP≤3.5, MW≤350) were docked, largely from a make-on-demand “lead-like” library (http://zinc15.docking.org) (Lyu, supra; Sterling, T. & Irwin, J. J. J Chem Inf Model 55, 2324-2337, 2015). Each molecule was fit into the orthosteric site of the 2.9 Å MT₁ structure (Stauch, B. et al. Nature 569, 284-288, 2019), prioritizing those interacting with key melatonin-binding residues (Stauch, supra; Johansson, L. C. et al. Nature 569, 289-292, 2019) such as Q181^{ECL2} and N162⁴⁶⁰, which hydrogen bond with 2-phenylmelatonin in the MT₁ and MT₂ crystal structures. Each molecule was sampled in an average of 3445 orientations, and for each orientation an average of 485 conformations-over 72 trillion receptor-ligand complexes in total were assessed. Each complex was evaluated for receptor complementarity using the physics-based scoring function in DOCK3.7 (Coleman, R. G. et al., PLoS One 8, e75992, 2013). Diverse molecules within the top scoring molecules were identified by clustering the top 300,000 docking-ranked molecules by topological similarity, using an ECFP4-based Tanimoto coefficient (Tc) cutoff of 0.5, resulting in 65,323 clusters. Molecules that were similar to known MT₁ and MT₂ ligands from ChEMBL23 (Bento, A. P. et al. Nucleic Acids Res 42, D1083-1090, 2014) (ECFP4 Tc≥0.38) were eliminated to prioritize new chemotypes (FIG. 1). The new ligands dock to capture melatonin-like interactions observed in the crystal structures (Stauch, B. et al., Nature 569, 284-288, 2019; Johansson, L. C. et al. Nature 569, 289-292, 2019). Examples include the hydrogen-bond interactions with N162^{4.61} made by the methoxy group of 2-phenylmelatonin and in the docked models by esters (ZINC92585174), pyridines ('9032), and benzodioxoles (ZINC301472854); stacking with F179^{ECL2} by an indole in the crystal structure with the melatonin analog, but by benzoxazines ('0041), thiophenes ('3878), and furans (ZINC433313647) in the new ligands; and the hydrogen bond with Q181^{ECL2} that can be made not by an acetamide, as in melatonin, but by an ester or even a pyridine in the docked ligands (FIG. 2). The new ligands also dock to make interactions not found in the MT receptor structures, including hydrogen bonds with T178^{ECL2}, N255^{6.52}, A158^{4.56}, G104^{3.29}, and F179^{ECL2} (FIGS. 1C, 1E, 7A-7E).

Docking a library of 150 million diverse, make-on-demand chemotypes found multiple molecules, topologically unrelated to known MTR ligands, with picomolar and nanomolar activities on the melatonin receptors. Each of the fifteen docking hits, each synthesized de novo, represented a different scaffold, ensuring chemical diversity. The chemical novelty of these molecules translated functionally, conferring MTR type selectivity and the rarely reported inverse agonism. Compounds '3384 and '7447 were among the first MT₁-selective ligands with activity in vivo. This activity was not only potent (EC₅₀ of 30 μg/kg), but unexpectedly phenocopied the behavioral effects of melatonin in circadian phase shift, suggesting previously unknown signaling control for the MT₁ type.

The best scoring molecules from each of the top 10,000 clusters were inspected for engagement with residues that recognize 2-phenylmelatonin in the MT₁ and MT₂ crystal structures (Stauch, supra; Johansson, supra) and for new polar partners in the MT₁ site. In the docked complexes, these included hydrogen bonds with T178^{ECL2}, N255^{6.52}, and with the backbone atoms of A158^{4.56}, G104^{3.29}, and F179^{ECL2}. Conformationally strained molecules and those with unsatisfied hydrogen bond donors were deprioritized (Wang, S. et al. Science 358, 381-386, 2017; Irwin, J. J. & Shoichet, B. K. J Med Chem 59, 4103-4120, 2016). Of the best-scoring molecules in each prioritized cluster, all related cluster molecules were inspected and the one that best fit these criteria was chosen. Ultimately, 40 high-scoring molecules with ranks ranging from 16 to 246,437, or the top 0.00001% to top 0.1% of the over 150 million docked, were selected for synthesis and experimental testing. Of the 38 molecules successfully synthesized (a 95% fulfillment rate), 15 had activity at either or both of the two melatonin receptors in in vitro functional assays (Table 1, FIG. 2), a hit rate of 39% (defined as number-active/number-physically-tested).

The 15 active molecules were characterized by high potency and a range of efficacies, including both agonism and inverse agonism, consistent with the emphasis on chemotype novelty. This novelty is supported quantitatively by their low ECFP4 Tc values to such known ligands, ranging from 0.2 to 0.33, suggesting that new scaffolds are being explored (Muchmore, S. W. et al. J Chem Inf Model 48, 941-948, 2008) visually by comparison of the new ligands to their closest analogs among the knowns (Table 1), and pharmacologically by their diverse potencies and functional activities (FIG. 2). Consistent with docking against an agonist-bound MT₁ structure, four of the new ligands were MT₁-selective agonists (FIGS. 5A, 5B), with EC₅₀ values in the 2 to 6 μM range, and without detectable MT₂ activity up to 30 M: ZINC419113878, ZINC182731037, ZINC353044322, and ZINC151209032. Strikingly, ZINC159050207, although only 2-fold selective for the MT₁ v. the MT₂ receptor type, is a low nanomolar agonist at MT₁ with an EC₅₀ value of lnM (pEC50=9.00±0.15, Emax(%)=99±1), and is among the most potent agonists found directly from a docking screen, and indeed from most types of GPCR compound screens (FIGS. 1B, 1C) (Table 1, FIGS. 5C, 5D). Admittedly, many molecules were just as active at the MT₂ receptor, or even selective for it. For instance, ZINC580731466 is almost 100-fold selective for the MT₂ receptor (Table 1, FIGS. 5C, 5D), while the 0.47 nM ZINC482850041 is 25-fold selective for the MT₂ receptor (FIGS. 1B, 1D). ZINC603324490 is a 600 nM strong inverse agonist of the MT₂ receptor without substantial MT₁ efficacy in that concentration range (FIGS. 1E, 1F, 5E, 5F), while ZINC157665999 is a 50 nM inverse agonist of the MT₂ but a 10 μM agonist of MT₁(FIGS. 1F, 5E, 5F, Table 1). Thus, whereas the initial docking against the MT₁ structure succeeded in finding new, potent chemotypes, and some of these were type selective, they were just as likely to prefer the MT₂ type as the MT₁ type. This attests to both the strengths and weaknesses of chemotype novelty as a strategy for compound prioritization, and to the need for optimization.

We optimized twelve of these ligands, each a different scaffold, representing the range of functions found from the initial docking hits. We selected analogs of the initial hits from the make-on-demand library that had favorable docking scores and poses (Table 2). Compounds were considered analogs if they had the same scaffold (Bemis, G. W. & Murcko, M. A. J Med Chem 39, 2887-2893, 1996) as the initial docking hit, or if they had ECFP4-based Tc values >0.5 to that molecule; overall, several thousand analogs were docked and evaluated. Of these, 131 were synthesized and tested in two rounds; as few as one and as many as 30 analogs were synthesized and tested for each lead series. Ninety of these analogs had activity at either or both MT₁ or MT₂ melatonin receptors at concentrations ≤10 μM (Table 1, FIG. 6), and five of the twelve scaffolds saw improved potency. While this structure-based analoging could reliably find potent ligands, their activity as agonists, inverse agonists, MT₁- or MT₂ type selective compounds, often flipped with small structural changes (FIGS. 7A-7E). An example is '0041, one of the most G protein-biased compounds tested at MT₂, while its close analog ZINC608506688 showed the most ß-arrestin bias (Kenakin, T. Pharmacol Rev 71, 267-315, 2019) at MT₂ (FIG. 7F, Table 3).

TABLE 1
Active molecules from the initial docking screen

| | Clus- | | | | |
|---|---|---|---|---|---|
| | ter | | | | |
| | rank | | | | |
| | (global | MT₁pEC50 | MT₂pEC50 | | Nearest ChEMBL MT₁/MT₂ |
| Compound | rank) | (% Emax) | (% Emax) | Tc^{a} | Ligand |
| 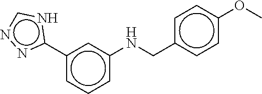 ZINC157665999^{b} | 166 (197) | 4.89 ± 0.38 (63 ± 6) | Inverse 7.29 ± 0.16 (Inverse 90 ± 16) | 0.33 | 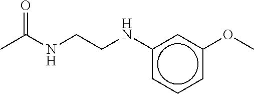 |
| 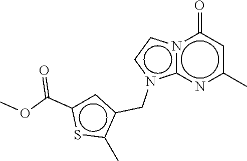 ZINC419113878^{b} | 395 (522) | 5.20 ± 0.08 (84 ± 4) | >5 | 0.22 | 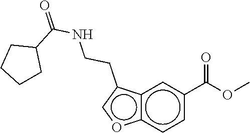 |
| 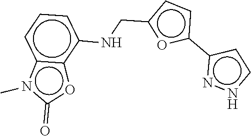 ZINC433313647^{b} | 874 (1241) | 6.81 ± 0.32 (42 ± 2) | 7.77 ± 0.02 (96 ± 5) | 0.19 | 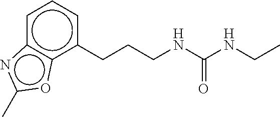 |
| 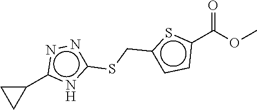 ZINC159050207^{b} | 1558 (2491) | 9.00 ± 0.15 (99 ± 1) | 8.70 ± 0.25 (83 ± 3) | 0.24 | 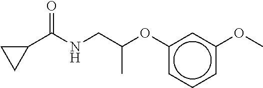 |
| 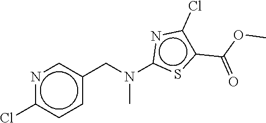 ZINC92585174^{b} | 1835 (3026) | 7.80 ± 0.17 (98 ± 1) | 7.68 ± 0.14 (74 ± 8) | 0.23 | 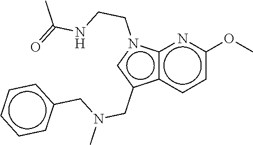 |
| 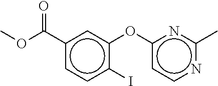 ZINC432154404 | 1848 (3050) | 6.63 ± 0.17 (95 ± 2) | 7.00 ± 0.17 (74 ± 4) | 0.27 | 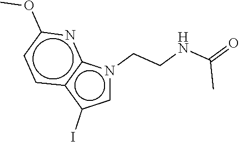 |
| 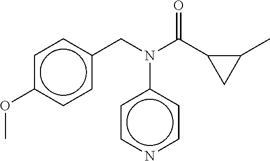 ZINC151209032^{b} | 1980 (3572) | 5.70 ± 0.11 (88 ± 4) | >5 | 0.31 | 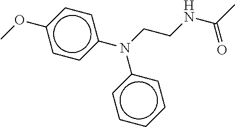 |
| 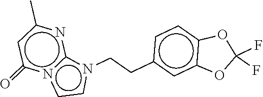 ZINC664088238^{b} | 2247 (3811) | >5 | 5.85 ± 0.06 (75 ± 8) | 0.20 | 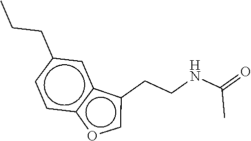 |
| 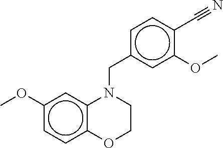 ZINC442850041^{b} | 4122 (7856) | 7.91 ± 0.04 (99 ± 3) | 9.33 ± 0.33 (97 ± 2) | 0.29 | 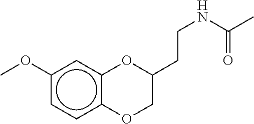 |
| 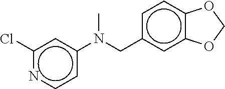 ZINC301472854^{b} | 5032 (10,028) | 6.03 ± 0.10 (95 ± 5) | 7.00 ± 0.21 (88 ± 6) | 0.26 | 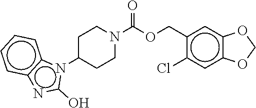 |
| 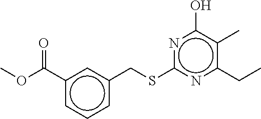 ZINC576887661 | 4160 (14,284) | 7.10 ± 0.19 (83 ± 0) | 7.28 ± 0.36 (68 ± 5) | 0.27 | 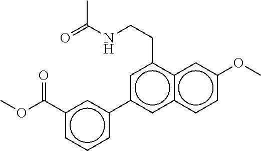 |
| 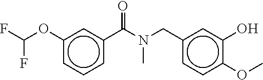 ZINC353044322^{b} | 5763 (28,239) | 5.48 ± 0.05 (87 ± 6) | >5 | 0.33 | 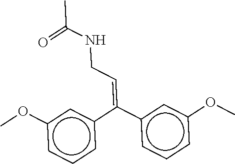 |
| 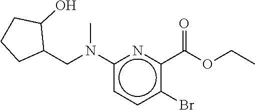 ZINC603324490^{b} | 7611 (53,881) | Inverse 5.92 ± 0.29 (Inverse 37 ± 5) | Inverse 6.20 ± 0.08 (Inverse 202 ± 30) | 0.27 | 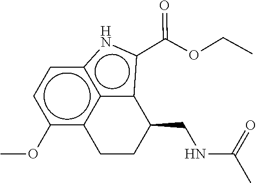 |
| 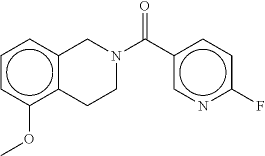 ZINC182731037^{b} | 7839 (17,180) | 5.30 ± 0.09 (82 ± 2) | >5 | 0.29 | 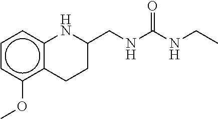 |
| 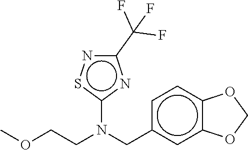 ZINC580731466 | 8502 (18,997) | 5.70 ± 0.13 (71 ± 3) | 7.55 ± 0.10 (98 ± 5) | 0.26 | 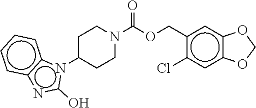 |

^{a}The ECFP4 Tanimoto similarity (T_{c}) to the most similar MT₁/MT₂ ligand in ChEMBL23.
^{b}Molecules chosen for optimization.

TABLE 2
Potent analogs from initial hits

| | | MT₁pEC50 | MT₂pEC50 |
|---|---|---|---|
| Initial Hit | Analog | (% Emax) | (% Emax) |
| 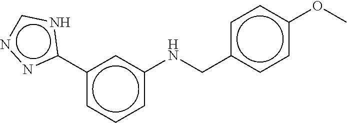 ZINC157665999 | 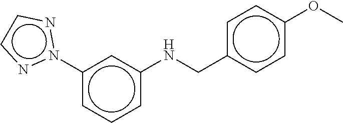 ZINC864032792 | 7.49 ± 0.04 (57 ± 3) | inverse 6.66 ± 0.08 (inverse 35 ± 5) |
| 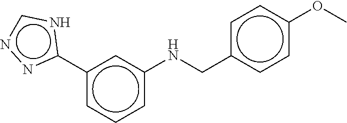 ZINC157665999 | 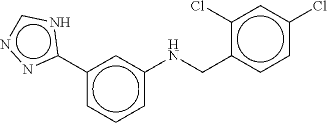 ZINC157676497 | Inverse 7.32 ± 0.05 (Inverse 60 ± 16) | Inverse 6.01 ± 0.30 (Inverse 62 ± 7) |
| 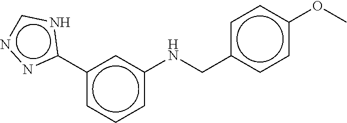 ZINC157665999 | 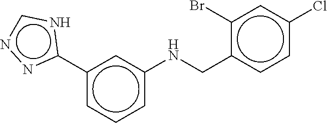 ZINC555417447 | Inverse 7.39 ± 0.10 (Inverse 62 ± 13) | Inverse 5.66 ± 0.10 (Inverse 84 ± 9) |
| 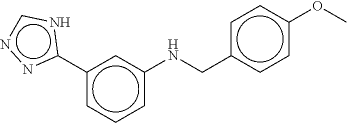 ZINC157665999 | 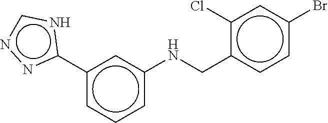 ZINC516666069 | Inverse 7.05 ± 0.03 (Inverse 42 ± 8) | Inverse 5.58 ± 0.04 (Inverse 118 ± 7) |
| 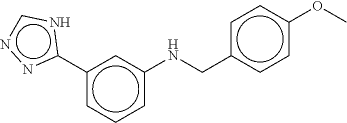 ZINC157665999 | 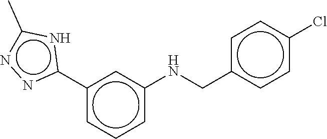 ZINC37781620 | Inverse 7.90 ± 0.90 (inverse 19 ± 4) | Inverse 6.20 ± 0.10 (inverse 120 ± 16) |
| 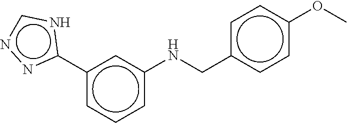 ZINC157665999 | 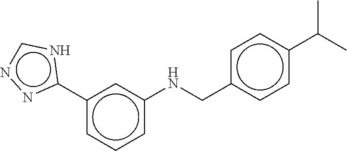 ZINC157673384 | Inverse 7.68 ± 0.09 (inverse 47 ± 12) | Inverse 6.18 ± 0.04 (inverse 153 ± 14) |
| 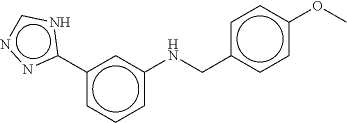 ZINC157665999 | 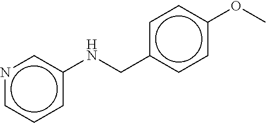 ZINC5586789 | 6.81 ± 0.72 (37 ± 8) | 8.07 ± 0.15 (51 ± 3) |
| 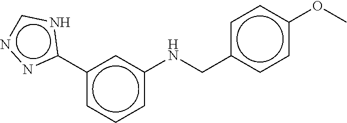 ZINC157665999 | 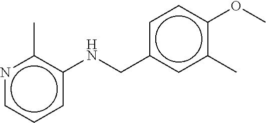 ZINC91496083 | 4.65 ± 0.44 (66 ± 2) | 7.15 ± 0.17 (106 ± 9) |
| 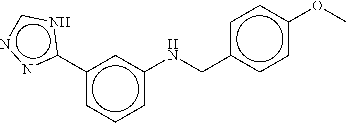 ZINC157665999 | 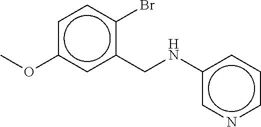 ZINC128734226 | 6.83 ± 0.17 (79 ± 3) | 8.15 ± 0.09 (89 ± 3) |
| 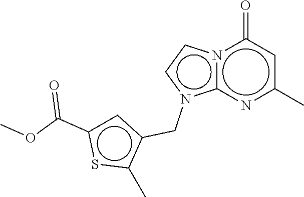 ZINC419113878 | 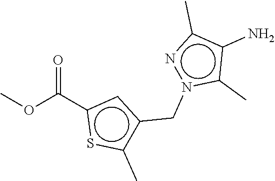 ZINC602421874 | 4.70 ± 0.11 (51 ± 3) | 5.35 ± 0.10 (66 ± 7) |
| 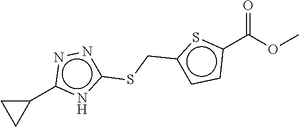 ZINC159050207 | 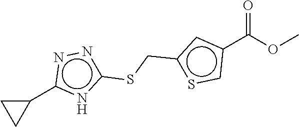 ZINC713465976 | 7.75 ± 0.22 (101 ± 0) | 8.23 ± 0.11 (94 ± 3) |
| 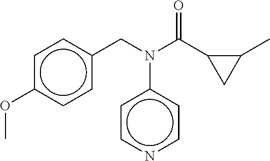 ZINC151209032 | 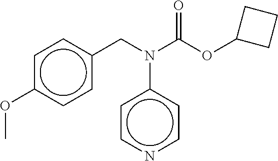 ZINC497291360 | 7.05 ± 0.10 (92 ± 2) | 7.48 ± 0.05 (75 ± 5) |
| 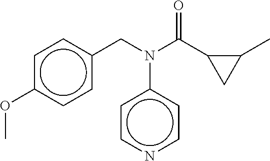 ZINC151209032 | 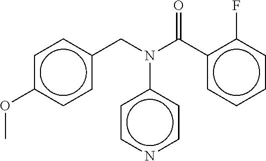 ZINC151192780 | 5.18 ± 0.22 (54 ± 4) | 7.13 ± 0.12 (95 ± 5) |
| 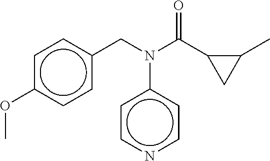 ZINC151209032 | 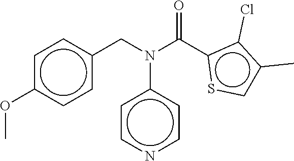 ZINC485552623 | >5 | 5.80 ± 0.06 (107 ± 5) |
| 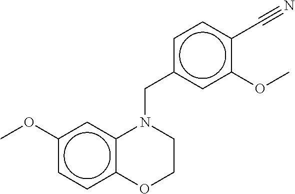 ZINC442850041 | 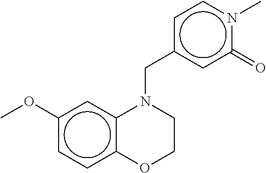 ZINC608506688 | 9.78 ± 0.13 (99 ± 1) | 8.60 ± 0.10 (89 ± 3) |
| 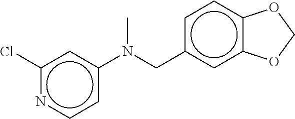 ZINC301472854 | 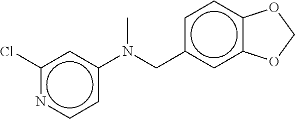 ZINC223593565 | 6.40 ± 0.18 (86 ± 4) | 6.45 ± 0.20 (58 ± 5) |

TABLE 3
Biased ligands from docking screening

| | Gi | β-arrestin | | |
|---|---|---|---|---|
| | Log(Emax/EC50) | Log(Emax/EC50) | ΔΔLog(Emax/EC50) | Bias* |
| Melatonin | 10.10 | 8.56 | | |
| (reference) | (9.85~10.3) | (8.3~8.8) | | |
| ZINC442850041 | 9.32 | 6.50 | −1.34 | 0.046 |
| | (9.20~9.56) | (6.2~6.7) | (−0.89~−1.8) | (0.016~0.13) |
| ZINC608506688 | 8.60 | 7.90 | 0.92 | 8.2 |
| | (8.3~8.8) | (7.7~8.2) | (0.46~1.37) | (2.9~23.4) |

Type-selective ligands were of particular interest as these are rare for the melatonin receptors. We investigated two MT₁-selective inverse agonists, compounds ZINC555417447 and ZINC157673384, and a selective MT₂ agonist, ZINC128734226, for their in vitro signaling profiles (FIG. 3), pharmacokinetics (Table 4), and efficacies in mouse models of circadian function (FIG. 4). As expected, '7447 and '3384 competed for 2-[¹²⁵I]-iodomelatonin binding to MT₁ receptors. Kᵢ values in the absence of GTP (304 nM and 938 nM, respectively) were enhanced by GTP (Kᵢ values of 7.5 and 63 nM, respectively), supporting their status as inverse agonists (FIGS. 3 and 8). Both '7447 and '3384 increased basal cAMP as predicted for inverse agonists, with EC₅₀ values of 41 and 21 nM at MT₁, selectivity for MT₁ over MT₂ of 50- and 30-fold, and inverse agonist efficacies of 62% and 47%, respectively (FIG. 3). The third molecule, '4226 was an MT2-selective agonist with an MT₂/MT₁ selectivity of 54 in 2-[¹²⁵I]-iodomelatonin binding assays; in isoproterenol-stimulated cAMP inhibition, the agonist had an EC₅₀ of 6.3 nM at MT₂ (FIGS. 3 and 8). In mouse pharmacokinetic (PK) studies, all three molecules had favorable brain and plasma concentrations, with brain/plasma ratios ranging from 1.4 to 3.0, suggesting substantial CNS permeability. Plasma half-lives (t_{1/2}) ranged from 0.27 to 0.32 hours (Table 4), similar to melatonin (Dubocovich, M. L. et al. Pharmacol Rev 62, 343-380, 2010). The PK profiles of these compounds increase their usefulness as tools to study the in vivo modulation of circadian rhythm.

TABLE 4
Pharmacokinetics of three MTR type-selective ligands

| | | | | | CL | | Brain/ |
|---|---|---|---|---|---|---|---|
| | pIC₅₀ (Emax %) | Cₘₐₓ(ng/ | AUC | T1/2 | (mL/ | Vss | Plasma |
| Compound | pEC₅₀ (IA) | mL) | (hr*ng/mL) | (hr) | min/kg) | (L/kg) | Ratio |
| 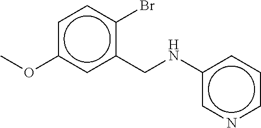 ZINC128734226 MT₂-selective agonist | pIC₅₀ MT1-6.8 (48%) MT2-8.2 (80%) | 1922.8 | 282.1 | 0.29 | 117.9 | 1.11 | 1.58 (30′) |
| 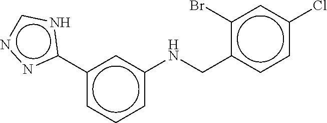 ZINC555417447 MT₁-select inv. ago | pEC₅₀ MT1-7.3 (IA) MT2-6.0 (IA) | 1948.6 | 494.5 | 0.27 | 67.11 | 1.11 | 3.03 (30′) |
| 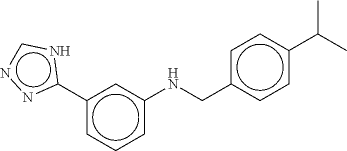 ZINC157673384 MT₁-selective inverse ago | pEC₅₀ MT1-7.7 (IA) MT2-6.5 (IA) | 1299.6 | 563.8 | 0.32 | 58.48 | 1.38 | 1.43 (30′) |

As the new chemotypes translated into new in vitro activities, the new in vitro activity translated into new in vivo activities. The behavioral effect of the inverse agonists, in particular, was unanticipated. Rather than acting opposite to melatonin, '7447 and '3384 phenocopied melatonin when dosed at dusk (CT10) (FIG. 4), advancing the circadian phase at concentrations similar to that of exogenous melatonin, and with similar efficacies. In previous studies, nonselective melatonin receptor agonists, like agomelatine and ramelteon, act similarly to melatonin (Rawashdeh, O. et al., Chronobiol Int 28, 31-38, 2011; Van Reeth, O. et al., Brain Res 762, 185-194, 1997), while nonselective and MT₂-preferring antagonists have no effect (Dubocovich, M. L. et al., FASEB J 12, 1211-1220, 1998). The MT₁ basis for the effect of the new inverse agonists is supported by the ablation of the activity of '7447 in MT₁KO mice (FIG. 4), maintenance of its activity in MT₂KO mice, and by the selectivity of these inverse agonists against 318 other GPCRs (FIG. 9). The ability of these MT₁-selective inverse agonists to phenocopy nonselective agonists may reveal an unanticipated signaling role for the MT₁ receptor, such as feedback control via pre-synaptic inhibition, by functional selectivity, or by acting to inhibit the G_{i/o}-dependent pathways, while leaving another, currently unknown pathway, uninhibited or even stimulated. While such mechanisms are admittedly speculative for the MT₁ receptor, each has precedence in other GPCRs (Betke, K. M. et al., Prog Neurobiol 96, 304-321, 2012; Kenakin, T. Br J Pharmacol 168, 554-575, 2013). Irrespective, the new selective MT₁ inverse agonists provide the field with tools to probe this signaling, arguably for the first time.

The abilities of the three molecules were further examined to advance or delay circadian phase in vivo. This was measured by the onset of mouse running wheel activity in constant darkness, a widely used model (Dubocovich, M. L. & Markowska, M. Endocrine 27, 101-110, 2005; Lewy, A. J., Cold Spring Harb Symp Quant Biol 72, 623-636, 2007; Dubocovich, M. L., et al., FASEB J 12, 1211-1220, 1998). Surprisingly, when dosed at dusk (CT10), both MT₁-selective inverse agonists ('7447 and '3384) phase-advanced circadian wheel running, an effect characteristic of nonselective agonist drugs like ramelteon (Rawashdeh, O. et al., Chronobiol Int 28, 31-38, 2011) and agomelatine (Van Reeth, O. et al. Brain Res 762, 185-194, 1997) and of the endogenous agonist melatonin (FIGS. 4A-4E). As inverse agonists, these compounds expected to display the opposite effect, delaying rather than advancing circadian rhythm (Ersahin, C., et al., Eur J Pharmacol 439, 171-172 (2002); Masana, M. I. et al. J Pharmacol Exp Ther 302, 1295-1302 (2002); Soares, J. M., Jr., et al., J Pharmacol Exp Ther 306, 694-702, 2003). Both '7447 and '3384 advanced the onset of activity by approximately 1 hour at 0.9 μg/mouse (˜30 μg/Kg), a dose equivalent to the EC₅₀ of melatonin in this behavioral assay (FIG. 4E). At a higher dose (30 μg/mouse; 1 mg/Kg), both compounds advanced the onset of running wheel activity with an amplitude equivalent to, or even larger than that of melatonin (Dubocovich, supra) at this circadian time (CT10). By contrast, the MT₂-selective agonist '4226 did not significantly affect circadian phase at either 0.9 or 30 μg/Kg doses (FIG. 4E), consistent with the MT₁-mediated nature of this effect (Dubocovich, M. L., et al., J Pineal Res 39, 113-120, 2005).

To investigate whether the unexpected behavioral effects of the inverse agonist reflect off-target activities of either the molecules themselves, or of their possible metabolites, both '7447 and '3384, as well as the MT₂ agonist '4226, were screened against 318 other GPCRs in agonist mode. Neither of the two inverse agonists had significant activity against any of the 318 receptors. However, the MT2 agonist '4426 did have some activity on 5HT2c (FIG. 9). Consistent with the high selectivity of the MT₁ inverse agonists, their phase-advance of running wheel activity at dusk (CT10) was eliminated in MT₁ knockout (MT₁KO) mice, but not in MT₂KO mice, consistent with an MT₁-based effect (Hudson, R., et al., Neuropsychopharmacol. S267-S267 (Nature Publishing Group, Macmillan Building, 4 Crinan St, London N1 9XW, England) (FIGS. 4F, 10A-10F). Also, while melatonin delays phase when given at dawn (CT2) (Benloucif, S. & Dubocovich, M. L., J Biol Rhythms 11, 113-125, 1996; Lewy, A. J., et al., Chronobiol Int 19, 649-658 (2002), the inverse agonist '7447 had no such effect (FIG. 4G, 10G-10L). Thus, while the MT₁-selective inverse agonists unexpectedly phenocopy melatonin's activity when dosed at dusk, their effects resembled those of nonselective antagonists when dosed at dawn.

The in vivo activity of the two MT₁-selective inverse agonists was further measured in a mouse “jet-lag” model. Mice were subjected to an abrupt six hour advance of dark onset, and molecules were dosed at the new dark onset time for three consecutive days, and the rate of re-entrainment to the new light-dark cycle was measured (FIGS. 4H-4L). At 30 μg/mouse melatonin accelerated re-entrainment to the new cycle (consistent with the effect of melatonin on human jet-lag). Conversely, at the same dose both inverse agonists '7447 and '3384 decelerated re-entrainment, as measured by the number of days to adapt to the new cycle. Unlike their agonist-like effects on circadian phase when dosed at dusk, the inverse agonists had the opposite effects of melatonin, resembling the activities of nonselective antagonist/inverse agonists such as luzindole (Adamah-Biassi, et al., The FASEB J 26, 1042-1045, 2012). Consistent with selective activity at MT₁, the effect of '7447 was eliminated in the MT₁KO mouse (FIGS. 4N, 4I-4J, 11A-11F), but not in the MT₂KO mice. This is in agreement with the ablated effect of melatonin on re-entrainment in MT₁KO mice (Dubocovich et al., J Pineal Res 39, 113-120, 2005), potentially reflecting the mediation of re-entrainment through MT₁ via exogenous melatonininergic ligands, and the rapid desensitization of the MT₂ receptor (Gerdin, M. J. et al., J Pharmacol Exp Ther 304, 931-939, 2003). Re-entrainment was decelerated in MT₂KO mice, as observed previously (Pfeffer, M. et al., Chronobiol Int 29, 415-429, 2012), even without the inverse agonist ('7447). This reflects the activation of the MT₂ type in the WT animals by endogenous melatonin in this behavior. The greater deceleration on dosing with the inverse agonist ('7447) in the MT₂KO mice may reflect the additional effect of the ligand tested as an inverse agonists on the MT₁ receptor.

From a large library docking screen emerged a wide range of new chemotypes for the melatonin receptors (FIG. 2), with new signaling and new pharmacology. We noted that docking a library of 150 million diverse, make-on-demand chemotypes found multiple molecules, topologically unrelated to known MTR ligands, with picomolar and nanomolar activities on the melatonin receptors. Each of the fifteen docking hits, each synthesized de novo, represented a different scaffold, ensuring chemical diversity. The chemical novelty of these molecules translated functionally, conferring MTR type selectivity and the rarely reported inverse agonism. Compounds '3384 and '7447 are among the first MT₁-selective ligands with activity in vivo. We yet further noted that this activity was not only potent (EC₅₀ of 30 μg/Kg), but unexpectedly phenocopied the behavioral effects of melatonin in circadian phase shift, suggesting previously unknown signaling control for the MT₁ type.

Most previously-known MT receptor ligands resemble melatonin itself, with variants of its indole ring, its methoxy and its ethyl acetamide side chains (Zlotos, D. P., Curr Med Chem 19, 3532-3549, 2012) (FIG. 2). The docking-derived ligands typically have different moieties at equivalent positions, rarely resembling melatonin except in their modeled receptor interactions. Thus, the indole-like ring of melatonin analogs can be replaced with pyrimidines, pyridines, and triazoles, the methoxy group can be replaced with an alkyl, while the ethyl acetamide side chain can be replaced by alkyl aromatics, activated ethers, or heteroaromatics (FIG. 2). Notwithstanding these differences, the new ligands dock to capture melatonin-like interactions observed in the crystal structures (Stauch, B. et al., Nature 569, 284-288, 2019; Johansson, L. C. et al., Nature 569, 289-292, 2019). Examples include the hydrogen-bond interactions with N162^{4.61} made by the methoxy group of 2-phenylmelatonin and in the docked models by esters (ZINC92585174), pyridines ('9032), and benzodioxoles (ZINC301472854); stacking with F179^{ECL2} by an indole in the crystal structure with the melatonin analog, but by benzoxazines ('0041), thiophenes ('3878), and furans (ZINC433313647) in the new ligands; and the hydrogen bond with Q181^{ECL2} that can be made not by an acetamide, as in melatonin, but by an ester or even a pyridine in the docked ligands (FIG. 2). The new ligands also dock to make interactions not found in the MT receptor structures, including hydrogen bonds with T178^{ECL2}, N255^{6.52}, A158^{4.56}, G104^{3.29}, and F179^{ECL2} (FIG. 1C, 1E, 7A-7E).

As the new chemotypes translated into new in vitro activities, the new in vitro activity translated into new in vivo activities. The behavioral effect of the inverse agonists, in particular, was unanticipated. Rather than acting opposite to melatonin, '7447 and '3384 phenocopied melatonin when dosed at dusk (CT10) (FIG. 4), advancing the circadian phase at concentrations similar to that of exogenous melatonin, and with similar efficacies. In previous studies, nonselective melatonin receptor agonists, like agomelatine and ramelteon, act similarly to melatonin (Rawashdeh, O., et al., Chronobiol Int 28, 31-38, 2011; Van Reeth, O. et al., Brain Res 762, 185-194, 1997), while nonselective and MT₂-preferring antagonists have no effect (Dubocovich, M. L., et al., FASEB J 12, 1211-1220, 1998). The MT₁ basis for the effect of the new inverse agonists is supported by the ablation of the activity of '7447 in MT₁KO mice (FIG. 4), maintenance of its activity in MT₂KO mice, and by the selectivity of these inverse agonists against 318 other GPCRs (FIG. 9). The ability of these MT₁-selective inverse agonists to phenocopy nonselective agonists may reveal an unanticipated signaling role for the MT₁ receptor, such as feedback control via pre-synaptic inhibition, by functional selectivity, or by acting to inhibit the G_{i/o}-dependent pathways, while leaving another, currently unknown pathway, uninhibited or even stimulated. While such mechanisms are admittedly speculative for the MT₁ receptor, each has precedence in other GPCRs (Betke, K. M., et al., Prog Neurobiol 96, 304-321, 2012; Kenakin, T., Br J Pharmacol 168, 554-575, 2013). Irrespective, the new selective MT₁ inverse agonists provide the field with tools to probe this signaling for the first time.

To conclude, 38 high-ranking molecules were synthesized de novo and tested for activity, revealing both agonists and inverse agonists in the 470 μM to 6 μM range at both melatonin receptors. Subsequent structure-based optimization led to two selective MT inverse agonists that were tested for effects on circadian behavior in a mouse model. Unexpectedly, these inverse agonists advanced the mouse circadian clock by about 1.3-1.5 hrs, an agonist-like effect. This circadian effect was eliminated in MT₁-knock-out mice, but not MT₂-knockout mice, consistent with an MT₁ selective mechanism.

Provided below is a list of potent MT₂ receptor agonists (Table 5) and MT₁ receptor inverse agonists (Table 6).

TABLE 5
MT₂ receptor agonists

| | MT₁ | | MT₂ | |
|---|---|---|---|---|
| | pEC50 | Emax | pEC50 | Emax |
| Compound | (M) | (% MT) | (M) | (% MT) |
| 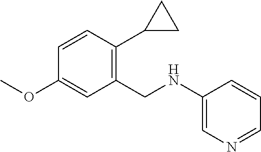 [SMILES: COc1ccc(C2CC2)c(CNc2cccnc2)c1] | ND | ND | 8.74 ± 0.22 | 75 ± 5 |
| 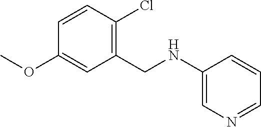 [SMILES: COc1ccc(Cl)c(CNc2cccnc2)c1] Z3670677760 | 7.37 ± 0.05 | 93 ± 2 | 8.68 ± 0.09 | 103 ± 7 |
| 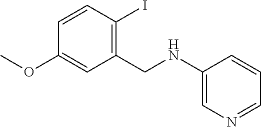 [SMILES: COc1ccc(I)c(CNc2cccnc2)c1] Z3670677756 | 7.29 ± 0.08 | 82 ± 6 | 8.58 ± 0.08 | 105 ± 18 |
| 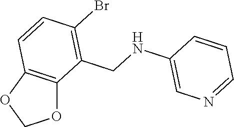 [SMILES: Brc1ccc2c(c1CNc1cccnc1)OCO2] Z3670677785 | 7.29 ± 0.09 | 89 ± 4 | 8.53 ± 0.24 | 100 ± 15 |
| 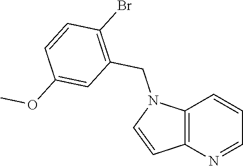 [SMILES: COc1ccc(Br)c(Cn2ccc3ncccc32)c1] Z3670677782 | 7.44 ± 0.10 | 93 ± 2 | 8.67 ± 0.05 | 101 ± 7 |
| 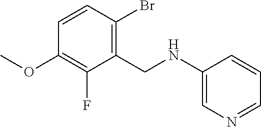 [SMILES: COc1ccc(Br)c(CNc2cccnc2)c1F] Z3670677772 | 6.20 ± 0.19 | 70 ± 6 | 7.52 ± 0.31 | 70 ± 14 |
| 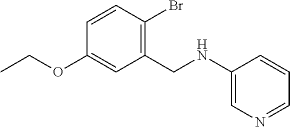 [SMILES: CCOc1ccc(Br)c(CNc2cccnc2)c1] Z3670677767 | 6.72 ± 0.08 | 80 ± 4 | 7.83 ± 0.02 | 48 ± 12 |
| 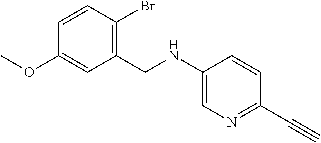 [SMILES: C#Cc1ccc(NCc2cc(OC)ccc2Br)cn1] Z3670677770 | 6.08 ± 0.30 | 67 ± 12 | 7.96 ± 0.09 | 91 ± 2 |

TABLE 6
MT₁ receptor inverse agonists

| | MT₁ | Emax | MT₂ | |
|---|---|---|---|---|
| | pEC50 | (% | pEC50 | Emax |
| Compound | (M) | basal) | (M) | (% basal) |
| 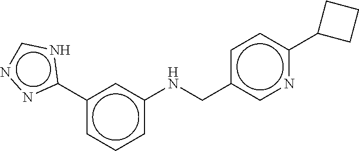 Z3668902468 | Inverse 7.76 ± 0.15 | Inverse 114 ± 23 | Inverse 7.33 ± 0.01 | Inverse 95 ± 19 |
| 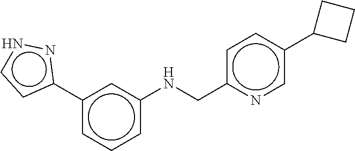 Z3668902474 | Inverse 7.17 ± 0.15 | Inverse 95 ± 17 | Inverse 6.63 ± 0.2 | Inverse 78 ± 36 |
| 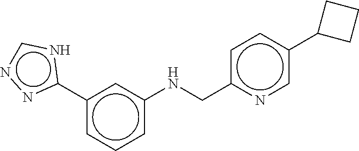 Z3668902470 | Inverse 7.59 ± 0.12 | Inverse 105 ± 25 | Inverse 7.05 ± 0.14 | Inverse 204 ± 25 |
| 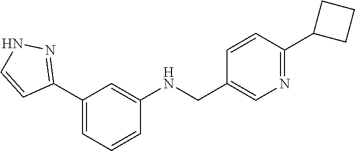 [SMILES: c1cc(NCc2ccc(C3CCC3)nc2)cc(-c2cc[nH]n2)c1] Z3668902476 | inverse 7.12 ± 0.13 | Inverse 30 ± 9 | ND | ND |
| 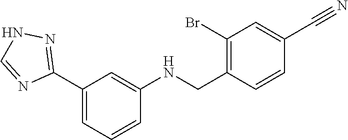 [SMILES: N#Cc1ccc(CNc2cccc(-c3nc[nH]n3)c2)c(Br)c1] Z3464201813 | inverse 7.80 ± 0.06 | inverse 122 ± 21 | inverse 6.41 ± 0.08 | inverse 55 ± 13 |
| 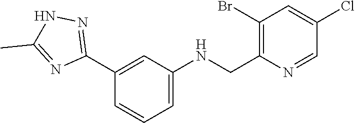 [SMILES: Cc1nc(-c2cccc(NCc3ncc(Cl)cc3Br)c2)n[nH]1] Z3668902489 | Inverse 6.61 ± 0.10 | Inverse 47 ± 7 | ND | ND |
| 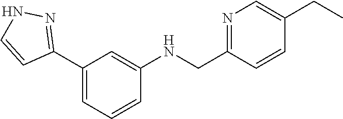 [SMILES: CCc1ccc(CNc2cccc(-c3cc[nH]n3)c2)nc1] Z3668902485 | Inverse 6.96 ± 0.24 | Inverse 31 ± 13 | Inverse 6.51 ± 0.11 | Inverse 119 ± 43 |
| 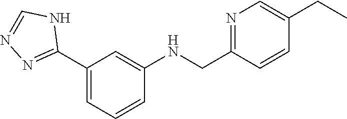 [SMILES: CCc1ccc(CNc2cccc(-c3nnc[nH]3)c2)nc1] Z3668902484 | Inverse 7.70 ± 0.05 | Inverse 61 ± 9 | Inverse 6.99 ± 0.10 | Inverse 153 ± 9 |
| 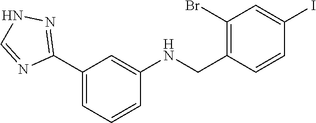 [SMILES: Brc1cc(I)ccc1CNc1cccc(-c2nc[nH]n2)c1] Z3464201812 | Inverse 7.28 ± 0.13 | Inverse 62 ± 11 | Inverse 5.74 ± 0.51 | Inverse 60 ± 16 |
| 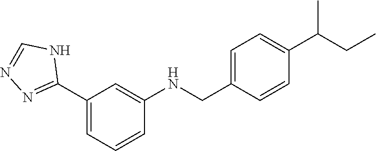 [SMILES: CCC(C)c1ccc(CNc2cccc(-c3nnc[nH]3)c2)cc1] Z3464201821 | Inverse 7.32 ± 0.07 | Inverse 45 ± 12 | Inverse 6.05 ± 0.09 | Inverse 96 ± 37 |
| 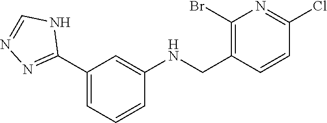 [SMILES: Clc1ccc(CNc2cccc(-c3nnc[nH]3)c2)c(Br)n1] Z3415429060 | Inverse 6.4 ± 0.1 | Inverse 63 ± 9 | Inverse 6.09 ± 0.04 | Inverse 134 ± 23 |
| 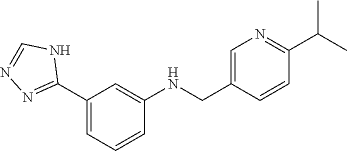 [SMILES: CC(C)c1ccc(CNc2cccc(-c3nnc[nH]3)c2)cn1] Z3464201827 | Inverse 7.98 ± 0.21 | Inverse 52 ± 9 | Inverse 7.23 ± 0.06 | Inverse 162 ± 22 |
| 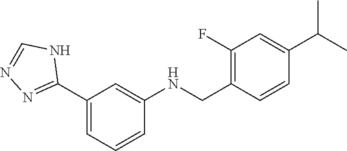 [SMILES: CC(C)c1ccc(CNc2cccc(-c3nnc[nH]3)c2)c(F)c1] Z3464201819 | Inverse 7.1 ± 0.12 | Inverse 85 ± 22 | Inverse 5.85 ± 0.09 | Inverse 100 ± 14 |
| 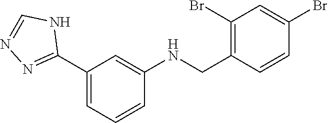 [SMILES: Brc1ccc(CNc2cccc(-c3nnc[nH]3)c2)c(Br)c1] Z3464201811 | Inverse 7.34 ± 0.09 | Inverse 77 ± 14 | Inverse 5.63 ± 0.06 | Inverse 87 ± 4 |
| 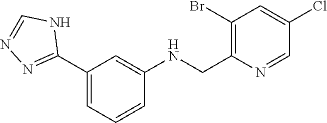 [SMILES: Clc1cnc(CNc2cccc(-c3nnc[nH]3)c2)c(Br)c1] Z3415429068 | Inverse 6.83 ± 0.12 | Inverse 74 ± 21 | Inverse 5.8 ± 0.13 | Inverse 83 ± 14 |

It is understood that the examples described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of increasing melatonin type 2 (MT₂) receptor activity in a subject in need thereof, the method comprising administering to said subject an effective amount of a compound of formula (I): 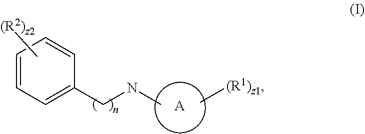
or a pharmaceutically acceptable salt thereof, wherein:
n is and integer from 0 to 5;
z1 is an integer from 0 to 2;
z2 is an integer from 0 to 5;
ring A is a substituted or unsubstituted aryl or heteroaryl;
R¹ is independently halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl; and
X¹ and X² are independently halogen.

2. A method of treating depression in a subject in need thereof, the method comprising administering to said subject an effective amount of a compound of formula (I): 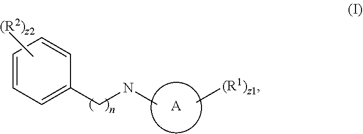
or a pharmaceutically acceptable salt thereof, wherein:
n is and integer from 0 to 5;
z1 is an integer from 0 to 2;
z2 is an integer from 0 to 5;
ring A is a substituted or unsubstituted aryl or heteroaryl;
R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —N, —NR^{1A}R^{1B}, —C(O)R^{1A}, —C(O)—OR^{1A}, —C(O)NR^{1A}R^{1B}, —C(O)NH—NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl; and
X¹ and X² are independently halogen.

3. A method of treating an MT₂ receptor-related condition in a subject in need thereof, the method comprising administering to said subject an effective amount of a compound of formula (I): 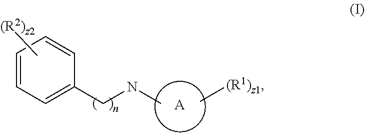
or a pharmaceutically acceptable salt thereof, wherein:
n is and integer from 0 to 5;
z1 is an integer from 0 to 2;
z2 is an integer from 0 to 5;
ring A is a substituted or unsubstituted aryl or heteroaryl;
R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl; and
X¹ and X² are independently halogen.

4. The method of claim 1, wherein the MT₂ receptor-related condition is somnipathy.

5. (canceled)

6. The method of claim 1, wherein ring A is a substituted or unsubstituted phenyl or a substituted or unsubstituted pyridinyl.

7. (canceled)

8. (canceled)

9. The method of claim 1, wherein the compound having formula (Ia) or (Ic): 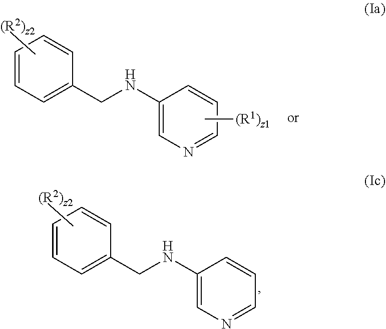
or a pharmaceutically acceptable salt thereof.

10. (canceled)

11. (canceled)

12. The method of claim 1, wherein R¹ is —C≡C—.

13. The method of claim 1, wherein R² is independently halogen, —OR^{2A}, or substituted or unsubstituted cycloalkyl.

14. (canceled)

15. (canceled)

16. (canceled)

17. (canceled)

18. (canceled)

19. (canceled)

20. The method of claim 1, wherein the compound is:

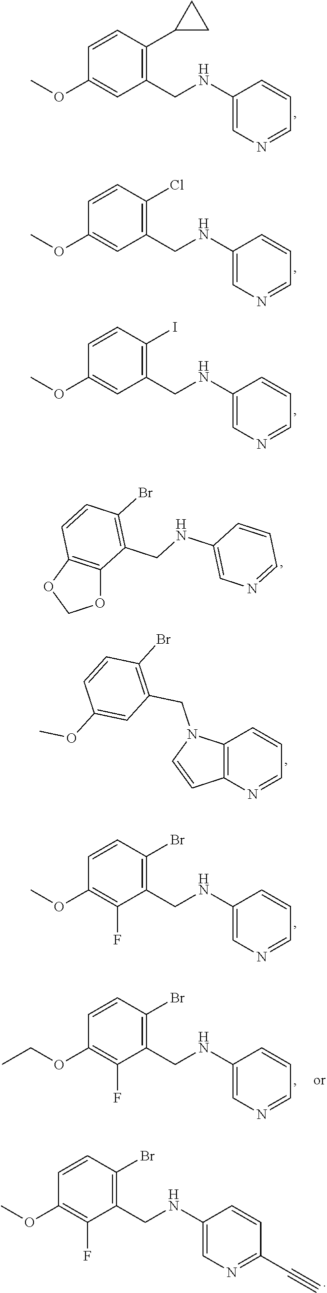

21. A method of advancing circadian phase comprising administering to a subject in need thereof an effective amount of an inverse agonist of melatonin type 1 (MT₁) receptor of formula (II): 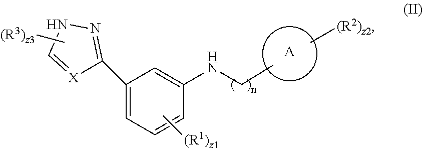
or a pharmaceutically acceptable salt thereof, wherein:
n is and integer from 0 to 5;
z1 is an integer from 0 to 2;
z2 is an integer from 0 to 5;
z3 is an integer from 0 to 2;
X is —N or —CH;
ring A is a substituted or unsubstituted aryl or heteroaryl;
R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —N, —NR^{1A}R^{1B}, —C(O)R^{1A}, —C(O)—OR^{1A}, —C(O)NR^{1A}R^{1B}, —C(O)NR¹NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl;
R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{3A} and R^{3B} substituents bonded to the same nitrogen atom may optionally be joined to form a substituted or unsubstituted heterocycloalkyl or substituted or unsubstituted heteroaryl; and
X¹, X² and X³ are independently halogen.

22. (canceled)

23. A method of decreasing of MT₁ receptor activity in a subject in need thereof, the method comprising administering to said subject an effective amount of a compound of formula (II): 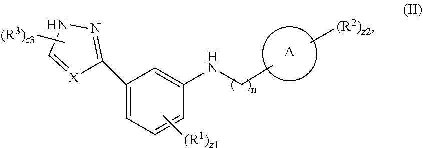
or a pharmaceutically acceptable salt
thereof, wherein:
n is and integer from 0 to 5;
z1 is an integer from 0 to 2;
z2 is an integer from 0 to 5;
z3 is an integer from 0 to 2;
X is —N or —CH;
ring A is a substituted or unsubstituted aryl or heteroaryl;
R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl;
R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{3A} and R^{3B} substituents bonded to the same nitrogen atom may optionally be joined to form a substituted or unsubstituted heterocycloalkyl or substituted or unsubstituted heteroaryl; and
X¹, X² and X³ are independently halogen.

24. A method of treating an MT₁ receptor related condition in a subject in need thereof, the method comprising administering to said subject an effective amount of a compound of formula (II): 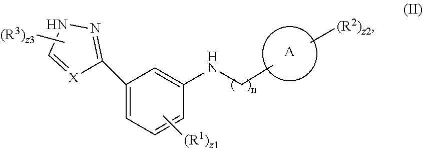
or a pharmaceutically acceptable salt thereof, wherein:
n is and integer from 0 to 5;
z1 is an integer from 0 to 2;
z2 is an integer from 0 to 5;
z3 is an integer from 0 to 2;
X is —N or —CH;
ring A is a substituted or unsubstituted aryl or heteroaryl;
R¹ is independently, halogen, —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NHNR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl
R^{1A} and R^{1B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{1A} and R^{1B} substituents bonded to the same nitrogen atom may optionally be joined to form a substituted or unsubstituted heterocycloalkyl or substituted or unsubstituted heteroaryl;
R^{2A} and R^{2B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{2A} and R^{2B} substituents bonded to the same nitrogen atom may optionally be joined to form a substituted or unsubstituted heterocycloalkyl or substituted or unsubstituted heteroaryl;
R^{3A} and R^{3B} are independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —Cl₃, —CHI₂, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —C(O)OH, —C(O)NH₂, —OH, —NH₂, —COOH, —CONH₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or
R^{3A} and R^{3B} substituents bonded to the same nitrogen atom may optionally be joined to form a substituted or unsubstituted heterocycloalkyl or substituted or unsubstituted heteroaryl; and
X¹, X² and X³ are independently halogen.

25. The method of claim 21, wherein the MT₁ receptor related condition is a circadian rhythm sleep-wake cycle disorder.

26. (canceled)

27. (canceled)

28. The method of claim 21, wherein ring A is a substituted or unsubstituted phenyl or a substituted or unsubstituted pyridinyl.

29. (canceled)

30. (canceled)

31. The method of claim 21 having formula (IIa): 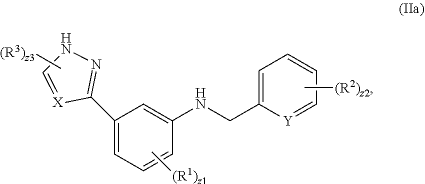
or a pharmaceutically acceptable salt thereof, wherein Y is —N or —CH.

32. (canceled)

33. (canceled)

34. (canceled)

35. The method of claim 21, wherein R² is independently halogen, —CN, substituted or unsubstituted alkyl, or substituted or unsubstituted cycloalkyl.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The method of claim 21, wherein the compound is:

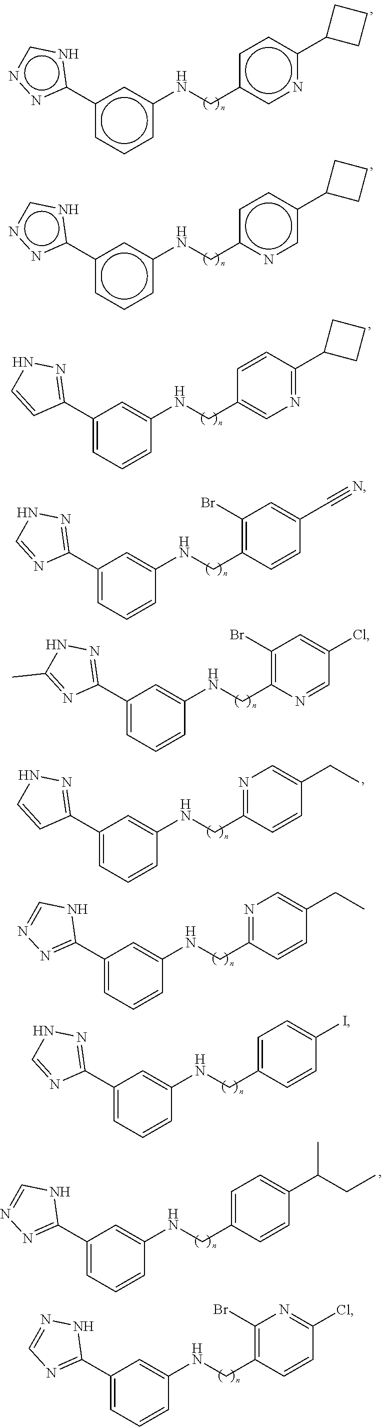

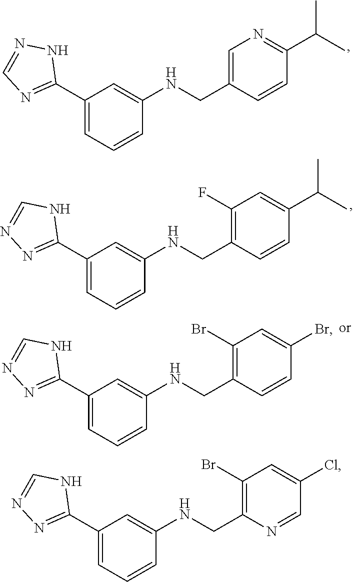 [SMILES: CC(C)c1ccc(CNc2cccc(-c3ncn[nH]3)c2)cn1 ; CC(C)c1ccc(CNc2cccc(-c3nnc[nH]3)c2)c(F)c1 ; Brc1ccc(CNc2cccc(-c3nnc[nH]3)c2)c(Br)c1 ; Clc1cnc(CNc2cccc(-c3nnc[nH]3)c2)c(Br)c1]

50. A compound selected from the group consisting of: 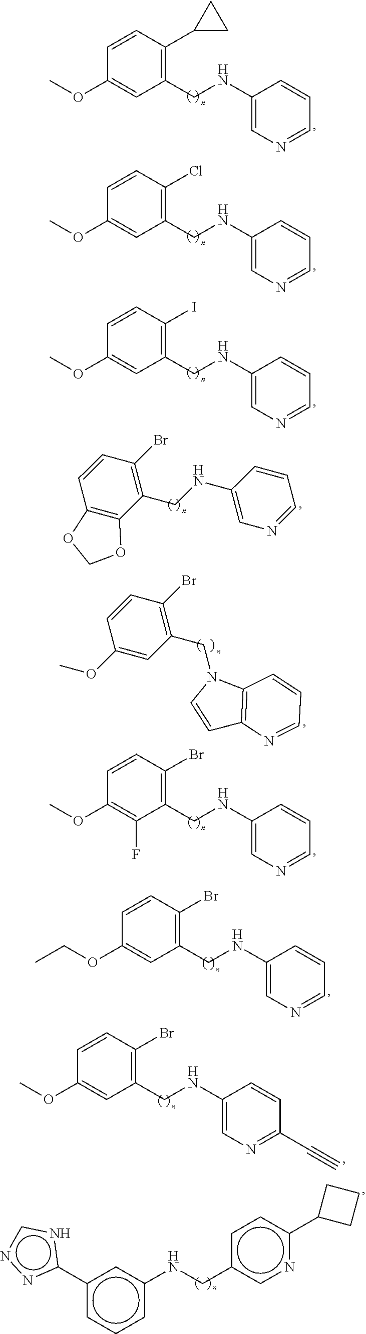 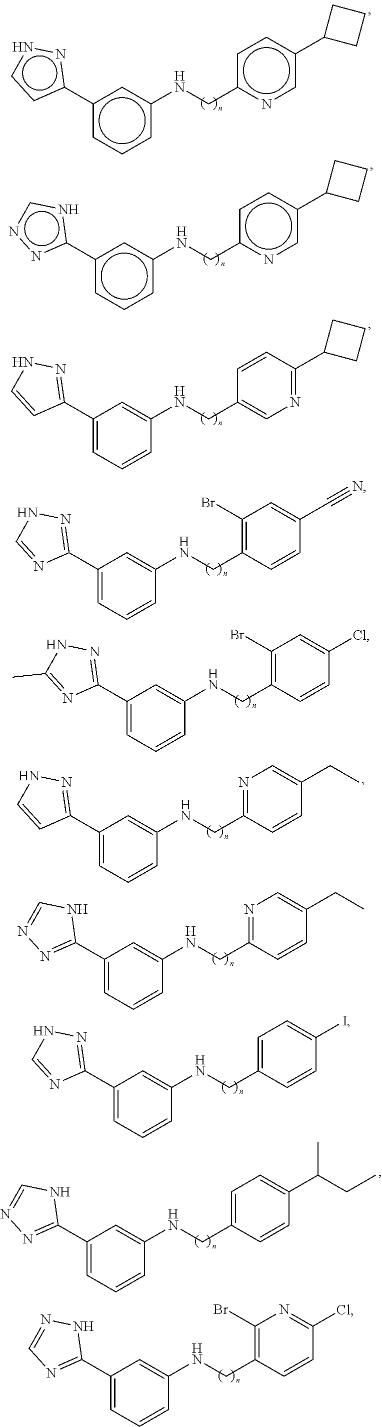 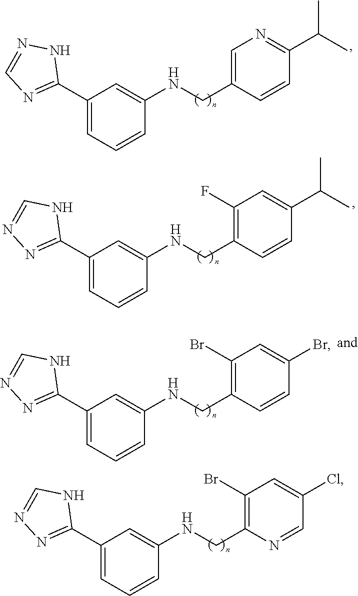
or a pharmaceutically acceptable salt thereof, wherein n=1.

51. (canceled)

52. (canceled)

53. The compound of claim 51, wherein R² is attached at the 1 or 4 position of the phenyl ring.

54. A pharmaceutical composition comprising the compound of claim 50, and a pharmaceutically acceptable carrier.
